SEQUENCE LISTING (27 sequences)

SEQ ID NO: 1 - protein, 214 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 2 - protein, 235 aa, Homo sapiens
GYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYG

SEQ ID NO: 3 - protein, 180 aa, Homo sapiens
MSDSGSYGQSGGEQQSYSTYGNPGSQGYGQASQSYSGYGQTTDSSYGQNYSGYSSYGQSYSQSYGGYENQKQSSYSQQPYNNQGQQQNMESSGSQGGRAPSYDQPDYGQQDSYDQQSGYDQHQGSYDEQSNYDQQHDSYSQNQQSYHSQRENYSHHTQDDRRDVSRYGEDNRGYGGSQGG

SEQ ID NO: 4 - protein, 340 aa, Homo sapiens
FNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGT

SEQ ID NO: 5 - protein, 149 aa, Homo sapiens
SNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 6 - protein, 86 aa, Homo sapiens
SKGPGRGDSPYSGRGDSPYSGRGDSPYSGRGDSPYSGRGDSPYSGRGDSPYSGRGDSPYSGRGDSPYSGRGDSPYSGRGDSPYSGY

SEQ ID NO: 7 - protein, 769 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRTSGSGSGSGSMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 8 - protein, 1011 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRTSGSGSGSGSMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGSGSMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 9 - protein, 732 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGGSGGTSMSDSGSYGQSGGEQQSYSTYGNPGSQGYGQASQSYSGYGQTTDSSYGQNYSGYSSYGQSYSQSYGGYENQKQSSYSQQPYNNQGQQQNMESSGSQGGRAPSYDQPDYGQQDSYDQQSGYDQHQGSYDEQSNYDQQHDSYSQNQQSYHSQRENYSHHTQDDRRDVSRYGEDNRGYGGSQGG

SEQ ID NO: 10 - protein, 974 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGGSGGTSMSDSGSYGQSGGEQQSYSTYGNPGSQGYGQASQSYSGYGQTTDSSYGQNYSGYSSYGQSYSQSYGGYENQKQSSYSQQPYNNQGQQQNMESSGSQGGRAPSYDQPDYGQQDSYDQQSGYDQHQGSYDEQSNYDQQHDSYSQNQQSYHSQRENYSHHTQDDRRDVSRYGEDNRGYGGSQGGGSGSMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 11 - protein, 787 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRTGSGSMGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYG

SEQ ID NO: 12 - protein, 1031 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRTGSGSMGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYGTSGSGSMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 13 - protein, 890 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGGSGGFNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGT

SEQ ID NO: 14 - protein, 1132 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGGSGGFNKSFGTPFGGGTGGFGTTSTFGQNTGFGTTSGGAFGTSAFGSSNNTGGLFGNSQTKPGGLFGTSSFSQPATSTSTGFGFGTSTGTANTLFGTASTGTSLFSSQNNAFAQNKPTGFGNFGTSTSSGGLFGTTNTTSNPFGSTSGSLFGPSSFTAAPTGTTIKFNPPTGTDTMVKAGVSTNISTKHQCITAMKEYESKSLEELRLEDYQANRKGPQNQVGAGTTTGLFGSSPATSSATGLFSSSTTNSGFAYGQNKTAFGTSTTGFGTNPGGLFGQQNQQTTSLFSKPFGQATTTQNTGFSFGNTSTIGQPSTNTMGLFGVTQASQPGGLFGTATNTSTGTGSGSMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 15 - protein, 699 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGSGSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 16 - protein, 943 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGSGSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMTSGSGSMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 17 - protein, 635 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGSSKGPGRGDSPYSGRGDSPYSGRGDSPYSGRGDSPYSGRGDSPYSGRGDSPYSGRGDSPYSGRGDSPYSGRGDSPYSGRGDSPYSGY

SEQ ID NO: 18 - protein, 879 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGSSKGPGRGDSPYSGRGDSPYSGRGDSPYSGRGDSPYSGRGDSPYSGRGDSPYSGRGDSPYSGRGDSPYSGRGDSPYSGRGDSPYSGYTSGSGSMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 19 - protein, 789 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRTSGSGSMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 20 - protein, 730 aa, Homo sapiens
MALPVTALLLPLALLLHAARPDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKGGGGSGGGGSGGGGSEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSAGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRTSGSGSGSGSMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 21 - protein, 748 aa, Homo sapiens
MALPVTALLLPLALLLHAARPDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKGGGGSGGGGSGGGGSEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSAGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRTGSGSMGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYG

SEQ ID NO: 22 - protein, 693 aa, Homo sapiens
MALPVTALLLPLALLLHAARPDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKGGGGSGGGGSGGGGSEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSAGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRTSGSGSMMSDSGSYGQSGGEQQSYSTYGNPGSQGYGQASQSYSGYGQTTDSSYGQNYSGYSSYGQSYSQSYGGYENQKQSSYSQQPYNNQGQQQNMESSGSQGGRAPSYDQPDYGQQDSYDQQSGYDQHQGSYDEQSNYDQQHDSYSQNQQSYHSQRENYSHHTQDDRRDVSRYGEDNRGYGGSQGG

SEQ ID NO: 23 - protein, 750 aa, Homo sapiens
MALPVTALLLPLALLLHAARPDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKGGGGSGGGGSGGGGSEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSAGSGSEQKLISEEDLGSTTTPAPRPPTPAPTIASQPLSLRPEACRPAAGGAVHTRGLDFACDIYIWAPLAGTCGVLLLSLVITLYCSRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRTSGSGSMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 24 - protein, 806 aa, Homo sapiens
METDTLLLWVLLLWVPGSTGDKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGSGSEQKLISEEDLGSTTTPAPRPPTPAPTIASQPLSLRPEACRPAAGGAVHTRGLDFACDFWVLVVVGGVLACYSLLVTVAFIIFWVSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRTSGSGSGSGSMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 25 - protein, 769 aa, Homo sapiens
METDTLLLWVLLLWVPGSTGDKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGSGSEQKLISEEDLGSTTTPAPRPPTPAPTIASQPLSLRPEACRPAAGGAVHTRGLDFACDFWVLVVVGGVLACYSLLVTVAFIIFWVSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRTSGSGSMMSDSGSYGQSGGEQQSYSTYGNPGSQGYGQASQSYSGYGQTTDSSYGQNYSGYSSYGQSYSQSYGGYENQKQSSYSQQPYNNQGQQQNMESSGSQGGRAPSYDQPDYGQQDSYDQQSGYDQHQGSYDEQSNYDQQHDSYSQNQQSYHSQRENYSHHTQDDRRDVSRYGEDNRGYGGSQGG

SEQ ID NO: 26 - protein, 824 aa, Homo sapiens
METDTLLLWVLLLWVPGSTGDKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGSGSEQKLISEEDLGSTTTPAPRPPTPAPTIASQPLSLRPEACRPAAGGAVHTRGLDFACDFWVLVVVGGVLACYSLLVTVAFIIFWVSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRTGSGSMGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYG

SEQ ID NO: 27 - protein, 826 aa, Homo sapiens
METDTLLLWVLLLWVPGSTGDKYLLPTAAAGLLLLAAQPAMAQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGGSGSEQKLISEEDLGSTTTPAPRPPTPAPTIASQPLSLRPEACRPAAGGAVHTRGLDFACDFWVLVVVGGVLACYSLLVTVAFIIFWVSRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRTSGSGSMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

(19) United States
(12) Patent Application Publication
(10) Pub. No.: US 2026/0224700 A1
(43) Pub. Date: Aug. 6, 2026

(54) CHIMERIC ANTIGEN RECEPTORS (CAR) WITH INTRINSICALLY DISORDERED REGIONS AND METHODS OF USE THEREOF

(71) Applicant: YALE UNIVERSITY, New Haven, CT (US)

(72) Inventors: Xiaolei SU, New Haven, CT (US); Xinyan ZHANG, New Haven, CT (US)

(21) Appl. No.: 19/149,103

(22) PCT Filed: Jan. 18, 2024
(86) PCT No.: PCT/US2024/012029

§ 371 Date: Jul. 18, 2025

Related U.S. Application Data
(60) Provisional application No. 63/480,488, filed on Jan. 18, 2023.
(60) Provisional application No. 63/481,737, filed on Jan. 26, 2023.

(51) Int. Cl. A61K 40/31; A61K 40/42; C07K 14/705; C07K 16/28; C07K 16/32

(52) U.S. Cl. CPC A61K 40/31; A61K 40/4205; A61K 40/4211; A61K 40/4212; C07K 14/7051; C07K 14/70517; C07K 14/70521; C07K 14/70578; C07K 16/2803; C07K 16/32

(57) ABSTRACT

Compositions and methods for improved Adoptive Cell Therapy (ACT) are provided. Compositions of CAR-T cells including CAR modified by fusion with one or more copies of a polypeptide including from between 50 and 500 amino acids derived from intrinsically disordered regions (IDRs) of proteins are described. CAR-IDR-T cells have improved condensation of CAR and T cell activation, and overall enhanced anti-tumor efficacy as compared with CAR-T cells lacking the IDR domain(s). In preferred embodiments, CAR-IDR fusion peptides include an IDR domain of any of SEQ ID NOs.1-6. The compositions and methods provide enhanced CAR-T cell therapy for cancer as well as other disease and disorders. Also disclosed are genetically modified cells and pharmaceutical compositions and methods of use thereof for treating subjects having diseases or disorders.

### CROSS-REFERENCE TO RELATED APPLICATIONS

This application claims the benefit of and priority to U.S. Provisional Application No. 63/480,488, filed Jan. 18, 2023, and U.S. Ser. No. 63/481,737 filed Jan. 26, 2023, each of which is hereby incorporated by reference in its entirety.

### STATEMENT REGARDING FEDERALLY SPONSORED RESEARCH

This invention was made with government support under GM138299 awarded by the National Institutes of Health. The government has certain rights in the invention.

### REFERENCE TO SEQUENCE LISTING

The Sequence Listing XML submitted as a file named “YU_8604_PCT_ST26.xml” and having a size of 42,656 bytes is hereby incorporated by reference pursuant to 37 C.F.R. § 1.834(c)(1).

### FIELD OF THE INVENTION

The invention is generally in the field of immunotherapy, and more particularly to improved chimeric antigen receptor (CAR) T cells with enhanced anti-tumor efficacy, and methods of making and using thereof.

### BACKGROUND OF THE INVENTION

Genetically engineered T cells represent a versatile and effective platform for cancer therapy. T cells equipped with Chimeric Antigen Receptors (CAR-Ts) have been tested in a variety of cancers and have achieved unprecedented success in treating blood malignancy (Ghobadi, A. C Curr Res Transi Med 66, 43-49, (2018); Lee, D. W. et al., Lancet 385, 517-528, (2015); Porter, D. L., et al., N Engl J Med 365, 725-733, (2011); Maldini, C. R., Ellis, G. I. & Riley, J. L. Nat Rev Immunol 18, 605-616, (2018)). As of 2022, there are six FDA approved CAR-T therapies for certain types of leukemia, lymphoma, and myeloma. These achievements have stimulated enthusiasm for developing new CAR-Ts targeting an expanding pool of cancerous, infectious, autoimmune, and fibrotic diseases (Aghajanian et al., 2019; Amor et al., 2020; Elinav et al., 2008; Ellebrecht et al., 2016; Hale et al., 2017; Leibman et al., 2017).

However, CAR T therapy is still limited by several major obstacles, including the risk of cytokine release syndrome, neurotoxicity, and limited signaling efficiency. Though derived from the T cell receptor (TCR), CARs typically display a much lower antigen sensitivity than TCR: a few hundred or more antigen molecules are required to activate a CAR T cell (Gudipati et al., 2020; Watanabe, K. et al., J Immunol 194, 911-920, (2015); Majzner, R. G. et al., Cancer Discov, doi:10.1158/2159-8290. CD-19-0945 (2020)) whereas a single peptide-loaded MHC molecule is sufficient to trigger the activation of a normal T cell (Irvine, D. J., Purbhoo, M. A., Krogsgaard, M. & Davis, M. M. Nature 419, 845-849, (2002); Huang, J. et al. Immunity 39, 846-857, (2013)). This low antigen sensitivity not only limits CAR-T's target to high antigen-expressing cancers, but also brought a challenge on maintaining sustained CAR-T activity against tumors. Since high antigen-expressing cancers downregulate their antigen levels to escape CAR-T's attacking, CAR T-treated patients experience an up to 60% of relapse, which is mostly associated with antigen loss (Park, J. H. et al. N Engl J Med 378, 449-459, (2018); Maude, S. L. et al. N Engl J Med 378, 439-448, (2018); Majzner, R. G. & Mackall, C. L. Cancer Discov 8, 1219-1226, (2018)). Therefore, there is a critical need to develop CAR T cells that can recognize low antigen-expressing cancer cells.

Mechanistically, the cause for low antigen sensitivity of CARs remains unclear. Because the binding of antigen to CAR is much tighter (with a Kd of nM to pM) than the binding of pMHC to TCR (with a Kd of μM). Therefore, the inability of CAR-Ts to be activated by low antigen-expressing cells more likely results from the signal processing mechanism downstream CAR rather than the binding of CAR to antigen.

It is an object of the invention to provide CAR T cells with enhanced antigen sensitivity and sustained CAR-T activity against tumors.

It is also an object of the invention to provide enhanced CAR T cells having improved anti-tumor efficacy and methods thereof for treating proliferative diseases.

### SUMMARY OF THE INVENTION

Compositions and methods for improving the efficiency of CAR-T cells through engineering CARs for enhanced condensation of CARs at the cell surface and/or subsequent T cell activation have been developed. The disclosed compositions and methods are especially applicable to development of enhanced chimeric antigen receptor engineered T cell therapy (CAR-T).

Polypeptides including an amino acid sequence of an intrinsically disordered region (IDR) of a protein and an amino acid sequence that is heterologous to IDR are provided. Typically, the IDR includes between 50 and 500 amino acids. Polypeptides including an amino acid sequence including at least 70% and less than 100% sequence identity to any one of SEQ ID NOs:1-6 or functional fragment thereof; and a heterologous amino acid sequence that is heterologous to IDR are also described. In some embodiments, the polypeptide further includes the amino acid sequence of SEQ ID NO:1, or a functional fragment or variant thereof. In some embodiments, the polypeptide includes the amino acid sequence of SEQ ID NO:2 or a functional fragment or variant thereof. In some embodiments, the polypeptide includes one or more additional copies of the amino acid sequence of SEQ ID NO:3, or a functional fragment or variant thereof. In some embodiments, the polypeptide includes the amino acid sequence of SEQ ID NO:4 or a functional fragment or variant thereof. In some embodiments, the polypeptide includes the amino acid sequence of SEQ ID NO:5 or a functional fragment or variant thereof. In some embodiments, the polypeptide includes the amino acid sequence of SEQ ID NO:6 or a functional fragment or variant thereof.

Exemplary heterologous amino acid sequences further include one or more molecules such as a chimeric antigen receptor (CAR), protein transduction domain, fusogenic polypeptide, targeting signal, expression and/or purification tag. In some embodiments, the heterologous sequence includes a chimeric antigen receptor (CAR), and the amino acid sequence of the IDR is present within the extracellular or intracellular region of the CAR. CAR fusion proteins are modular in nature with several discrete domains fused together. The IDR can be inserted or addended to any of the existing domains of the CAR construct, preferably provided its addition does not eliminate the activity of the construct (e.g., to bind to antigen, transduce signal, active T cells, etc.)

In some embodiments, the heterologous sequence includes a chimeric antigen receptor (CAR), and the amino acid sequence of the IDR is contiguous with the carboxyl terminus of the CAR. In some embodiments, the heterologous sequence includes a chimeric antigen receptor (CAR) having an intracellular component of CD3 zeta, and the amino acid sequence of the IDR is contiguous with the intracellular component of CD3 zeta. In one embodiment, the heterologous sequence includes a chimeric antigen receptor (CAR) having a transmembrane domain of CD8a, cytosolic signaling domains or a fragment there of derived from CD28, 41BB, and CD3 zeta, and the amino acid sequence of the IDR is contiguous with the intracellular component of CD3 zeta. In another embodiment, the heterologous sequence includes a chimeric antigen receptor (CAR) having a transmembrane domain of CD28, cytosolic signaling domains or a fragment there of derived from CD28, 41BB, and CD3 zeta, and the amino acid sequence of the IDR is contiguous with the intracellular component of CD3 zeta. In a further embodiment, the heterologous sequence includes a chimeric antigen receptor (CAR) having a transmembrane domain of CD8a, cytosolic signaling domains or a fragment there of derived from CD28 and CD3 zeta, and the amino acid sequence of the IDR is contiguous with the intracellular component of CD3 zeta. In exemplary embodiments, the polypeptide has or includes the sequence of any one of SEQ ID NOs:7-27.

In some embodiments, the CAR is specific for an antigen selected from a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof. Exemplary CAR targets are selected from AFP, AKAP 4, ALK, Androgen receptor, B7H3, BCMA, Bcr Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD2, GD3, GloboH, GM3, gp100, GPC3, HER 2/neu, HER2, HMWMAA, HPV E6/E7, hTERT, Idiotype, IL12, IL13RA2, IM19, IX, LCK, Legumain, IgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR β, PLAC1, Polysialic acid, Proteinase3 (PRI), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, and XAGE. In some embodiments, the antigen is a cancer antigen selected from 4 1BB, 5T4, adenocarcinoma antigen, alpha fetoprotein, BAFF, B lymphoma cell, C242 antigen, CA 125, carbonic anhydrase 9 (CA IX), C MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD2, GD3 ganglioside, glycoprotein 75, GPNMB, HER2/neu, HGF, human scatter factor receptor kinase, IGF 1 receptor, IGF I, IgGI, L1 CAM, IL 13, IL 6, insulin-like growth factor I receptor, integrin α5β1, integrin αvβ33, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF β, TRAIL R1, TRAIL R2, tumor antigen CTAA16.88, VEGF A, VEGFR 1, VEGFR2, and vimentin. In exemplary embodiments, the CAR is anti-CD19, anti-CD22, and/or anti-HER2.

Nucleic acids including a nucleic acid encoding the polypeptides are also described. Exemplary nucleic acids include a nucleic acid encoding a polypeptide including a chimeric antigen receptor (CAR) and one or more of SEQ ID NOs:1-27. In some embodiments, the nucleic acid is RNA or DNA. In some embodiments, the nucleic acid is mRNA, and/or includes an expression control sequence(s), and/or is, or is encoded by a vector or a transposon. An exemplary vector is a viral vector, such as a lentiviral vector, an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector including a combination of any two or more of a Adeno-associated virus (AAV) vector, Herpes Simplex virus (HSV) vector, vesicular stomatitis (VSV) vector, or a human Bocavirus vector (hBoV). In some embodiments, the vector is a nucleic acid expression vector selected from a plasmid, a cosmid, and a replicon. In some embodiments, the nucleic acid includes one or more of a promotor, a protein transduction domain, fusogenic polypeptide, or targeting signal conjugated thereto.

Isolated cells including the polypeptides and/or nucleic acids are also provided. Exemplary cells include a T cell, hematopoietic stem cell (HSC), macrophage, natural killer cell (NK), or dendritic cell (DC). For example, in some embodiments, the T cell is a CD8+ T cell selected from effector T cells, memory T cells, central memory T cells, and effector memory T cells. In other embodiments, the T cell is a CD4+ T cell selected from Th1 cells, Th2 cells, Th17 cells, and Treg cells. Isolated cells including a polypeptide encoding (i) an amino acid sequence encoding a CAR; and (ii) an IDR optionally have an amino acid sequence of one or more of SEQ ID NOs:1-6, or a variant having at least 70%, 75%, 80%, 85%, 90%, 95%, or 99% identity to one or more of SEQ ID NOs:1-6, are also described. In some embodiments, the isolated cell includes a polypeptide including (i) an amino acid sequence encoding a CAR; and (ii) an amino acid sequence of one or more of SEQ ID NOs:1-6. In some embodiments, the amino acid sequence of any one of SEQ ID NOs: 1-6 is contiguous with the residue at the carboxyl terminus of the CAR.

Populations of cells derived by expanding an isolated cell including a polypeptide including: (i) an amino acid sequence encoding a CAR; and (ii) an amino acid sequence of one or more of SEQ ID NOs:1-6, or a variant having at least 75% identity to one or more of SEQ ID NOs:1-6 are also provided. In some embodiments, the amino acid sequence of one or more of SEQ ID NOs:1-6 is contiguous with the residue at the carboxyl terminus of the CAR.

Pharmaceutical compositions including (a) a population of cells expanded from an isolated cell including or expressing a polypeptide including: (i) an amino acid sequence encoding a CAR; and (ii) an amino acid sequence of one or more of SEQ ID NOs:1-6, or a variant having at least 75% identity to one or more of SEQ ID NOs:1-6; and (b) a pharmaceutically acceptable buffer, carrier, diluent or excipient are also provided.

Methods of treating a subject having a disease, disorder, or condition including administering to the subject an effective amount of a pharmaceutical composition of cells are also provided. Typically, the methods treat a subject having a disease, disorder, or condition associated with an elevated expression or specific expression of an antigen, by administering to the subject an effective amount of a T cell modified to include or express a polypeptide including: (i) an amino acid sequence encoding a CAR that targets the antigen; and (ii) an amino acid sequence of one or more of SEQ ID NOs:1-6, or a variant having at least 75% identity to one or more of SEQ ID NOs:1-6. In some embodiments, the cell was isolated from the subject having the disease, disorder, or condition. In other embodiments, the cell was isolated from a healthy donor.

Typically, the subject is a human, such as a human with a disease selected from cancer, an inflammatory disease, a neuronal disorder, HIV/AIDS, a diabetes, a cardiovascular disease, an infectious disease (including a viral, a protozoan, a bacterial disease, and an allergy), an autoimmune disease and an autoimmune disease, and a genetic disorder.

In some embodiments, the subject has cancer or has been identified as being at increased risk of developing cancer.

Methods of introducing a fusion peptide including an IDR domain into a cell, are also provided. Typically, the method includes the steps of introducing to the cell: (i) a vector or transposon or mRNA encoding a polypeptide encoding a fusion peptide including an IDR domain; and (ii) causing the polypeptide to be expressed in the cell.

Chimeric antigen receptors (CARs), including one or more IDR polypeptides (CAR-IDR) are described. In certain embodiments, the CAR-IDR targets CD19 and includes the amino acid sequence of any one of SEQ ID NOs:7-18. In a preferred embodiment, the CAR-IDR which targets CD19 and includes the amino acid sequence of any one of SEQ ID NOs:7-18 has increased activity in condensation of CAR and/or activation of T cells expressing the CAR-IDR compared to a control. A suitable control has the same CAR targeting CD19 but without fusion to an IDR, for example, an exemplary control has the amino acid sequence of SEQ ID NO:19. In other embodiments, the CAR-IDR targets CD22 and includes the amino acid sequence of any one of SEQ ID NOs:20-22. In a preferred embodiment, the CAR-IDR which targets CD22 and includes the amino acid sequence of any one of SEQ ID NOs:20-22 has increased activity in condensation of CAR and/or activation of T cells expressing the CAR-IDR compared to a control. An exemplary control has the amino acid sequence of SEQ ID NO:23. In further embodiments, the CAR-IDR targets HER2 and includes the amino acid sequence of any one of SEQ ID NOs:24-26. In a preferred embodiment, the CAR-IDR which targets HER2 and includes the amino acid sequence of any one of SEQ ID NOs:24-26 has increased activity in condensation of CAR and/or activation of T cells expressing the CAR-IDR compared to a control. An exemplary control has the amino acid sequence of SEQ ID NO:27.

Nucleic acids, including a nucleic acid sequence encoding the chimeric antigen receptor including an IDR polypeptide (CAR-IDR), that targets CD19, CD22, and/or HER2 are also described. In some embodiments, the nucleic acid is a vector or a transposon. Isolated cells including a CAR including an IDR polypeptide (CAR-IDR), that targets CD19, CD22, and/or HER2, or a nucleic acid encoding the polypeptide are also described. Populations of cells derived from expanding the isolated cells including the CAR-IDRs that target CD19, CD22, and/or HER2 are also provided, and pharmaceutical compositions including the populations of cells and a pharmaceutically acceptable buffer, carrier, diluent, or excipient are also described. Methods of treating a subject having a disease, disorder, or condition include administering to the subject an effective amount of the pharmaceutical composition. In some embodiments, the subject has cancer.

### BRIEF DESCRIPTION OF THE DRAWINGS

FIGS. 1A-1K depict construction of IDR CARs. FIG. 1A is a schematic of the CD19 CAR used in this study which contains an scFv targeting CD19 (FMC63), a CD8 hinge and transmembrane domain, an intracellular signaling domain composed of CD28, 41BB, and CD3ζ, an intrinsically disordered region (IDR), and superfolder GFP. FIGS. 1B-1I are dot plot graphs showing FACS analyses of total and cell surface expression of IDR CARs, as determined by GFP and surface staining of FMC63, showing T cells with No CAR (FIG. 1B); Control CAR (FIG. 1C); CAR with FUS IDR (FUS) (FIG. 1D); CAR with TAF15 IDR (TAF15) (FIG. 1E); CAR with EWS IDR (EWS) (FIG. 1F); CAR with Nup98 IDR (Nup98) (FIG. 1G); CAR with TDP43 IDR (TDP43) (FIG. 1H); and CAR with Synthetic IDR (SynIDR) (FIG. 1I), respectively. FIG. 1J is a graph showing the results of TIRF microscopy to determine condensation of IDR CARs, with clustering of CARs quantified as normalized variance (N=48-50 cells) of the fluorescence (0-1500) for each of Control, FUS, TAF15, EWS, Nup98, TDP43 and SynIDR, respectively. Shown are mean+std. All comparisons in this and following figures were made between individual IDR CARs and the control CAR (n.s. p>=0.05, * p<0.05, ** p<0.01, *** p<0.001). FIG. 1K is a graph showing the results of CAR-T cell proliferation with or without IL-2. Quantification of the absolute cell number in the culture with or without IL-2. N=3 independent experiments. Shown are mean+std.

FIGS. 2A-2Q depict IDR-enhanced cytotoxicity of CD19 CAR-T in vitro and in vivo. FIGS. 2A-2B are graphs of the expression of CD19 CARs on the T cell surface as determined by an antibody recognizing FMC63 and the expression of CD19 on Nalm6 cells, showing numbers of CARs for each of EWS IDR CAR (EWS), TAF15 IDR CAR (TAF15), FUS IDR CAR (FUS), Control CAR and No CAR, respectively (FIG. 2A); and numbers of CD19 on each of NalmCD19 high cells (34,517 molecules per cell), NalmCD19 low cells (1,470 molecules per cell), and Null, respectively; the average number of CD19 on each cell was determining by flow cytometry using a BD Quantibrite Kit (FIG. 2B). FIGS. 2C-2D are graphs of cytotoxicity of CD19 CAR-Ts to CD19 high Nalm6 cells (FIG. 2C), or CD19 low Nalm6 cells (FIG. 2D), showing specific lysis (%) for each Control (▪), FUS (▴), TAF15 (▾) and EWS (♦) CART cells, at ratios of 0.3:1 to 10:1, respectively, as reported by the luciferase assay. FIGS. 2E-2I are graphs showing production of cytotoxic factors by CAR-T. The control or IDR CAR-Ts were co-cultured with CD19-low Nalm6 cells for 1 day at an E:T of 3:1. The cytokines released into the culture media were quantified by flow cytometry using a LegendPlex assay kit. N=3 independent experiments. Shown are mean+std (* p<0.05, ** p<0.01, *** p<0.001). FIG. 2J is a schematic showing examination of the anti-tumor effect of CD19 IDR CAR-Ts in a mouse xenograft model. 1×10⁶ FLuc+CD19 low Raji B cells were engrafted into the immune-deficient NSG mice via tail vein. Three days later, 8×10⁶ CAR-T cells or PBS were injected into the mice via tail vein. Cancer progression was monitored by bioluminescent imaging. Blood was drawn at Day 16 to characterize T cell phenotypes. FIG. 2K-2O are graphs showing quantification of tumor progression in vivo revealed by bioluminescent imaging. Both the individual and averaged traces were shown. N=5 mice. Shown are mean+std (n.s. p>=0.05, *** p<0.001). FIG. 2P is a plot showing expression of T cell exhaustion markers during cancer progression. Blood was collected at day 16 post CAR-T infusion, stained with antibodies recognizing the exhaustion marker TIM3, LAG3, and PD1, and analyzed by flow cytometry. N=5 mice. Shown are mean+std (n.s. p>=0.05, *p<0.05, **p<0.01). FIG. 2Q is a plot showing quantification of T cells number in the blood. Blood was collected at day 16 post CAR-T infusion, stained with antibodies recognizing human CD3 and human CD45, and analyzed by flow cytometry. N=5 mice. Shown are mean+std (n.s. p>=0.05).

FIGS. 3A-3W depict IDRs enhancing cytotoxicity of HER2 CAR-T in vitro and in vivo. FIG. 3A is a schematic of the IDR CAR construct targeting HER2 (H3B1), a CD28 hinge and transmembrane domain, an intracellular signaling domain composed of CD28, 41BB and CD3ζ, and an intrinsically disordered region (IDR). The control CAR contains a GFP tag at the C-terminus so that the size and surface expression level of the control and IDR CARs can be maintained in a similar level over time. FIG. 3B is a graph of the expression of the control or IDR CAR targeting HER2 in human primary T cells. CAR expression was detected by recombinant HER2 proteins and measured by flow cytometry showing numbers of CARs for each of EWS IDR CAR (EWS), TAF15 IDR CAR (TAF15), FUS IDR CAR (FUS), Control CAR and Null, respectively (FIG. 3B). FIGS. 3C-3D are a pair of plots showing quantification of the HER2 level in K562 cells expressing high (102,797 molecules per cell) or low (5,703 molecules per cell) HER2, and in SKOV3 (77,679 molecules per cell) and HT29 (3,351 molecules per cell) cells. The average number of HER2 on each cell was determining by flow cytometry using a BD Quantibrite Kit. FIGS. 3E-3H are graphs of cytotoxicity of HER2 CAR-Ts to HER2 high or low K562, HT29 or NCK292 cells as reported by the luciferase assay, showing specific lysis (%) for each of Null, Control, FUS, TAF15 and EWS CART cells, at ratios of 0.3:1 to 10:1, respectively, for each of CAR-T:HER2-K562-high (FIG. 3E); CAR-T:HER2-K562-low (FIG. 3F); CAR-T:SKOV3 (FIG. 3G); and CAR-T:HT29 (FIG. 3H), CAR-T:NC1292 (FIG. 3W) respectively. FIGS. 3I-3M are graphs showing production of cytotoxic factors by CAR-T. The control or IDR CAR-Ts were co-cultured with HT29 cells for 1 day at an E:T of 3:1. The cytokines released into the culture media were quantified by flow cytometry using a LegendPlex assay kit. N=3 independent experiments. Shown are mean+std (n.s. p>=0.05, * p<0.05, ** p<0.01, ***p<0.001). FIG. 3N is a schematic showing examination of the anti-tumor effect of HER2 IDR CAR-Ts in a mouse xenograft model. 2.5×106 HT29 cells were engrafted subcutaneously into the immune-deficient NSG mice. Eight days later, 8×106 CAR-T cells were injected into the mice via tail vein, followed by a second injection of 4×106 CAR-T cells five days after. Cancer progression was monitored by measuring the tumor size using an electronic digital caliper. FIGS. 3O-3T are graphs showing quantification of cancer progression in vivo after CAR-T treatment. Both the averaged and individual traces were shown. N=5 mice. Shown are mean+std (*p<0.05). FIG. 3U is a plot showing quantification of T cell number in blood. Blood was drawn at Day 25, stained with antibodies recognizing human CD3 and CD45. The percentile of human T cells was measured by flow cytometry. N=5 mice. Shown are mean+std. FIG. 3V is a plot showing quantification of tumor-infiltrating T cells. At the end timepoint (the tumor size over 2000 mm3), tumors were dissected, digested, stained with antibodies recognizing human CD3 and CD45. The percentile of human T cells was measured by flow cytometry. N=5 mice. Shown are mean+std.

FIGS. 4A-4N depict IDRs enhanced the anti-tumor effect of CD22 CAR-T in vitro and in vivo. FIG. 4A is a schematic of the CD22 CAR used in this study. It contains an scFv targeting CD22 (RFB4, low signaling efficiency), a CD28 hinge and transmembrane domain, an intracellular signaling domain composed of CD28 and CD3ζ, and an intrinsically disordered region (IDR). The control CAR contained a GFP tag at the C-terminus so that the size and surface expression level of the control and IDR CARs were similar. FIG. 4B is a graph showing numbers of CARs for each of TAF15 CAR, EWS IDR CAR (EWS), FUS IDR CAR (FUS), Control CAR and Null, respectively. FIG. 4C is a plot showing quantification of the CD22 level in the wild-type Raji B (21,297 molecules per cell) and Nalm6 (8,376 molecules per cell). The average number of CD22 on each cell was determining by flow cytometry using a BD Quantibrite Kit. FIG. 4D is a graph of inhibition of cancer progression by CAR-Ts, showing Radiance (p/sec/cm2/sr) over time (Days post CAR-T treatment) for each of PBS (e), Control (n), FUS (A) and EWS (v), respectively. FIG. 4E is a bar graph of quantified flow cytometry analysis showing % Exhaustion markers of each of PD1, TIM3, and LAG3 on each of Control, FUS and EWS CAR-T cells, respectively, at day 19. N=5 mice. Shown are mean+std (n.s. p>=0.05, *p<0.05). FIGS. 4F-4G are graph showing cytotoxicity of CD22 CAR-T in vitro. The control or IDR CAR-Ts were co-cultured with Raji B (FIG. 4F) or Nalm6 (FIG. 4G) cells expressing luciferase (Fluc+) for 3 or 1 day, respectively, with an effector to target (E:T) ratio from 0.3:1 to 10:1. The percentile of lysed Naml6 cells were quantified by the luciferase assay and normalized to the group of Raji B or Nalm6 alone. N=3 independent experiments. Shown are mean+std (* p<0.05, **p<0.01, ***p<0.001). FIGS. 4H-4L are graphs showing production of cytotoxic factors by CAR-T. The control or IDR CAR-Ts were co-cultured with the wild-type Nalm6 cells for 1 day at an E:T of 1:1. The cytokines released into the culture media were quantified by flow cytometry using a LegendPlex assay kit. N=3 independent experiments. Shown are mean+std (n.s. p>=0.05, *p<0.05). FIG. 4M is a schematic showing examination of the anti-tumor effect of CD22 IDR CAR-Ts in a mouse xenograft model. 1×10⁶ FLuc+Nalm6-WT cells were engrafted into the immune-deficient NSG mice via tail vein. Three days later, 8×10⁶ CAR-T cells or PBS were injected into the mice via tail vein. Cancer progression was monitored by bioluminescent imaging. Blood was drawn at Day 10 and 19 to characterize T cell phenotypes. FIG. 4N shows quantification of T cells in the blood. Blood was collected at day 10 post CAR-T infusion, stained with antibodies recognizing human CD3 and human CD45, and analyzed by flow cytometry. N=5 mice. Shown are mean+std (*p<0.05, ** p<0.01).

FIGS. 5A-5L depict IDR promoted membrane-proximal signaling in CAR-T. FIG. 5A is a graph showing cell-cell conjugation between CAR-T and Nalm6 cells. CD19 CAR-Ts were co-cultured with CD19-low RajiB cells for 30 min at 37° C. with an E:T ratio of 1:1. Cell conjugation was examined by confocal microscopy. N=3 independent experiments. Shown are mean+std (n.s. p>=0.05). FIGS. 5B-5J are plots demonstrating phosphorylation kinetics of CD3ζ (pY142), LAT (pY171), and ERK (pT202/Y204). CAR-Ts were mixed with CD19-low Raji B cells at an E:T=1:1 at 37° C. Cells were fixed at 0, 5 or 15 min, stained and examined with flow cytometry. Displayed are traces for three independent experiments. Relative pCD3z, pLAT, and pERK was calculated by dividing the geometric mean of fluorescence at indicated time point to that at 0 min. FIG. 5K is a plot showing quantification of pCD3ζ at the CAR-T synapse. Normalized pCD3ζ was calculated by dividing the fluorescence of pCD3ζ to the fluorescence of CAR in the synapse. N=77-107 cells. Shown are mean+std (**p<0.01, *** p<0.001). FIG. 5L is a plot showing quantification of pLAT at the CAR-T synapse. Normalized pLAT was calculated by dividing the fluorescence of pLAT to the fluorescence of CAR in the synapse. N=79-105 cells. Shown are mean+std (***p<0.001).

FIGS. 6A-6I depict oligomerization by coiled-coil domain reduced CAR surface localization and CAR-T activation. FIG. 6A is a schematic of the coiled-coil (cc) CD19 CAR which contains an scFv targeting CD19 (FMC63), a CD8 hinge, a LAT transmembrane domain, an intracellular signaling domain composed of CD28, 41BB, and CD3ζ, and a coiled-coil domain. FIGS. 6B-6F are graphs showing expression of coiled-coil CARs. The localization of CAR on the T cell surface was detected via flow cytometry using an anti-FMC63 antibody. A GFP tag was fused on the C-terminus of each CAR to monitor the total expression of CAR. FIG. 6G is a plot showing cell-cell conjugation between CAR-T and Nalm6 cells. CAR-Ts were mixed with Naml6-H8 clone (expressing 137,795 CD19 molecules per cell) for 30 min at 37° C. with an E:T=1:1. Cell conjugation was monitored by confocal microscopy. N=3 independent experiments. Shown are mean+std (n.s. p>=0.05, * p<0.05, **p<0.01). FIGS. 6H-6I are plots showing activation of coiled-coil CAR-Ts assessed by CD69 expression and IFNγ release. CAR-Ts were co-cultured with Nalm6-H8 for 1 day at an E:T=1:1. CD69 expression was revealed by flow cytometry. IFNγ release was quantified by ELISA. N=3 independent experiments. Shown are mean+std (n.s. p>=0.05, *p<0.05, **p<0.01).

FIGS. 7A-7D depict that IDR do not trigger tonic signaling. FIGS. 7A-7B show graphs demonstrating activation of CD19 CAR-T in the presence and absence of antigen. CAR-Ts were cultured alone or co-cultured with the wild-type Raji B cells at E:T=10:1 for 1 day. Expression of CD69 was examined by flow cytometry. TNFα release was measured by ELISA. N=3 independent experiments. Shown are mean+std (n.s. p>=0.05, *p<0.05, **p<0.01). FIGS. 7C-7D are graphs showing activation of HER2 CAR-T in the presence and absence of antigen. CAR-Ts were stimulated by plate-coated HER2 protein for 1 day. Expression of CD69 was examined by flow cytometry. TNFα release was measured by ELISA. N=3 independent experiments. Shown are mean+std (n.s. p>=0.05, * p<0.05, **p<0.01, ***p<0.001).

FIGS. 8A-8P depict characterization of IDR CAR-Ts targeting CD19. FIG. 8A is a plot showing quantification of the CD19 level in Raji B cells expressing high (37,172 molecules per cell) or low (2,638 molecules per cell) level of CD19. The average number of CD19 on each cell was determined by flow cytometry using a BD Quantibrite Kit. FIGS. 8B-8C are graphs showing cytotoxicity of CD19 CAR-T against Raji B cells. The control or IDR CAR-Ts were co-cultured with CD19-high or low Raji B cells expressing luciferase (Fluc+) for 3 days with an effector to target (E:T) ratio from 1:1 to 10:1. The percentile of lysed Raji B cells were quantified by the luciferase assay and normalized to the group of Raji B alone. N=3 independent experiments. Shown are mean+std (*p<0.05, ** p<0.01). FIG. 8D demonstrates representative heat map illustrating cytotoxic factors released by CD19 CAR-Ts. The control or IDR CAR-Ts were co-cultured with CD19-low Nalm6 cells for 1 day at an E:T of 3:1. The cytokines released into the culture media were quantified by flow cytometry using a LegendPlex assay kit. FIGS. 8E-8G are representative flow cytometry plots showing T cell exhaustion markers. Blood was collected at day 16 post CAR-T infusion, stained with antibodies recognizing the exhaustion marker TIM3, LAG3, and PD1, and analyzed by flow cytometry. FIGS. 8H-8K are representative flow cytometry plots showing T cell population in the blood. Blood was collected at day 16 post CAR-T infusion, stained with antibodies recognizing human CD3 and human CD45. FIGS. 8L-80 are representative flow cytometry plots showing T cell differentiation in vivo. Blood was collected at day 16 post CAR-T infusion, stained with antibodies recognizing CD45RA and CD62L, and analyzed by flow cytometry. FIG. 8P is a graph showing quantification of T cell differentiation in vivo. N=5 mice. Shown are mean+std.

FIGS. 9A-9K depict characterization of IDR CAR-Ts targeting HER2. FIG. 9A is a plot of representative heat map illustrating cytotoxic factors released by HER2 CAR-Ts. The control or IDR CAR-Ts were co-cultured with HT29 cells for 1 day at an E:T of 3:1. The cytokines released into the culture media were quantified by flow cytometry using a LegendPlex assay kit. FIGS. 9B-9K are representative flow cytometry plots showing T cell population in the blood and tumor. Blood was draw at day 25 and tumors were dissected at day 48, digested, stained with antibodies recognizing human CD3 and CD45. The percentile of human T cells was measured by flow cytometry.

FIG. 10A-10M depict characterization of IDR CAR-Ts targeting CD22. FIG. 10A is plot of representative heat map illustrating cytotoxic factors released by CD22 CAR-Ts. The control or IDR CAR-Ts were co-cultured with the wild-type Nalm6 cells for 1 day at an E:T of 1:1. The cytokines released into the culture media were quantified by flow cytometry using a LegendPlex assay kit. FIGS. 10B-10E are representative flow cytometry plots showing T cell population in the blood. Blood was collected at day 10 post CAR-T infusion, stained with antibodies recognizing human CD3 and human CD45. FIGS. 10F-10H are representative flow cytometry plots showing T cell exhaustion markers. Blood was collected at day 19 post CAR-T infusion, stained with antibodies recognizing the exhaustion marker TIM3, LAG3, and PD1, and analyzed by flow cytometry. FIGS. 10I-10L are representative flow cytometry plots showing T cell differentiation in vivo. Blood was collected at day 10 post CAR-T infusion, stained with antibodies recognizing CD45RA and CD62L, and analyzed by flow cytometry. FIG. 10M shows quantification of T cell differentiation in vivo. N=5 mice. Shown are mean+std.

FIGS. 11A-11I depict phosphorylation kinetics of CD3ζ, LAT, and ERK. CAR-Ts were mixed with CD19-low Raji B cells at an E:T=1:1 at 37° C. Cells were fixed at 0, 5 or 15 min, stained with phospho-antibodies and examined with flow cytometry. Shown are representative flow plots.

FIGS. 12A-12F depict oligomerization by coiled-coil domain reduce CAR-T activation. FIGS. 12A-12B show representative flow cytometry plots showing CD69 expression in coiled-coil CAR-Ts. CAR-Ts were co-cultured with Nalm6-H8 for 1 day at an E:T=1:1. FIG. 12C is a graph showing cell-cell conjugation between CAR-T and Raji B cells. CAR-Ts were mixed with CD19-high Raji B cells for 30 min at 37° C. with an E:T=10:1. Cell conjugation was monitored by confocal microscopy. N=3 independent experiments. Shown are mean+std (* p<0.05, ***p<0.001). FIGS. 12D-12F are plots showing CD69 expression on coiled-coil CAR-Ts when engaged with Raji B cells. CAR-Ts were co-cultured with CD19-high Raji B cells for 1 day at an E:T=10:1. CD69 expression was revealed by flow cytometry. N=3 independent experiments. Shown on are mean+std (n.s. p>=0.05, ***p<0.001). Representative flow cytometry plots were displayed.

### DETAILED DESCRIPTION OF THE INVENTION

### I. Definitions

“Introduce” in the context of genome modification refers to bringing in to contact. For example, to introduce a gene editing composition to a cell is to provide contact between the cell and the composition. The term encompasses penetration of the contacted composition to the interior of the cell by any suitable means, e.g., via transfection, electroporation, transduction, gene gun, nanoparticle delivery, etc.

As used herein, “homologous” means derived from a common ancestor. For example, a homologous trait is any characteristic of organisms that is inherited by two or more species from a common ancestor species. Homologous sequences can be orthologous or paralogous. Homologous sequences are orthologous if they were separated by a speciation event: when a species diverges into two separate species, the divergent copies of a single gene in the resulting species are said to be orthologous. Orthologs, or orthologous genes, are genes in different species that are similar to each other because they originated from a common ancestor. Homologous sequences are paralogous if they were separated by a gene duplication event: if a gene in an organism is duplicated to occupy two different positions in the same genome, then the two copies are paralogous.

“Heterologous” means having a different relation, relative position, or structure. Thus, unless otherwise specified, heterologous includes joining or linking of two or more amino acid or nucleic acid sequences from that organism (e.g., species) that are not normally found joined or linked (e.g., together) as well as joining or linking of two or more amino acid or nucleic acid sequences from different species.

“Endogenous” refers to any material from or produced inside an organism, cell, tissue or system.

“Exogenous” refers to any material introduced from or produced outside an organism, cell, tissue or system.

The term “transmembrane domain” refers to any protein structure that is thermodynamically stable in a cell membrane, preferably a eukaryotic cell membrane. As used herein, the terms “extracellular domain” and “ectodomain” refer to any protein structure that is thermodynamically stable in outside of the cell membrane (i.e., in the extracellular space). As used herein, an “intracellular domain” refers to any protein structure that is thermodynamically stable in inside of the cell membrane (i.e., in the intracellular cytosol).

The term “Chimeric Antigen Receptor”, or alternatively “CAR”, refers to a set of polypeptides, typically two in the simplest embodiments, which when in an immune effector cell, provides the cell with specificity for a cancer cell, or other specific cell, and with intracellular signal generation. In exemplary embodiments, a CAR includes at least an antigen binding domain such as an extracellular binding domain, a transmembrane domain and a cytoplasmic signaling domain (also referred to as “an intracellular signaling domain”) including a functional signaling domain derived from a stimulatory molecule and/or costimulatory molecule. The term “antigen binding domain” is used in the context of a CAR to refer to the portion of a CAR that specifically recognizes and binds to an antigen of interest. The “antigen binding domain” of a CAR may be derived from a binding protein such as an antibody or fragment thereof. In some embodiments, the “binding domain” of a CAR is a single-chain variable fragment (scFv). In certain embodiments, the “binding domain” of a CAR includes the complementarity determining regions of a binding protein disclosed herein. In some embodiments, the stimulatory molecule is, or is derived from, the CD3ζ (zeta), also known as “zeta stimulatory domain”, associated with a T cell receptor complex. In some embodiments, the cytoplasmic signaling domain of the CAR further includes one or more functional signaling domains derived from at least one costimulatory molecule (e.g., 4-1BB (i.e., CD137), CD27 and/or CD28). In some embodiments, the CAR includes a chimeric fusion protein including an extracellular antigen binding domain, a transmembrane domain and an intracellular signaling domain including a functional signaling domain derived from a stimulatory molecule. In various embodiments, CARs are fusion proteins of single-chain variable fragments (scFv) fused to a CD3-zeta transmembrane domain. However, other intracellular signaling domains such as CD28, 41-BB and Ox40 may be used in various combinations to give the desired intracellular signal. Exemplary CAR-T cells include Axicabtagene ciloleucel (KTE-C19, Axi-cel), Tisagenlecleucel, Lisocabtagene Maraleucel (liso-cel; JCAR017).

The term “antigen” as used herein is defined as a molecule capable of being bound by an antibody or T-cell receptor. An antigen can additionally be capable of provoking an immune response. This immune response can involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which includes a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the disclosed compositions and methods includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid. In the context of cancer, “antigen” refers to an antigenic substance that is produced in a tumor cell, which can therefore trigger an immune response in the host. These cancer antigens can be useful as markers for identifying a tumor cell, which could be a potential candidate/target during treatment or therapy. There are several types of cancer or tumor antigens. There are tumor specific antigens (TSA) which are present only on tumor cells and not on healthy cells, as well as tumor associated antigens (TAA) which are present in tumor cells and on some normal cells. In some forms, the chimeric antigen receptors are specific for tumor specific antigens. In some forms, the chimeric antigen receptors are specific for tumor associated antigens. In some forms, the chimeric antigen receptors are specific both for one or more tumor specific antigens and one or more tumor associated antigens.

The terms “CD3ζ”, “CD3 zeta” or “CD3 eta” are used interchangeably to define the protein provided as GenBan Acc. No. BAG36664.1, or the equivalent residues from a non-human species. A “zeta stimulatory domain” or alternatively a “CD3-zeta stimulatory domain” is defined as the amino acid residues from the cytoplasmic domain of the zeta chain, or functional derivatives thereof, that are sufficient to functionally transmit an initial signal necessary for T cell activation.

The term “immune effector cell,” is used herein to refer to a cell that is involved in an immune response (e.g. promotion of an immune effector response). Examples of immune effector cells include T cells, e.g., alpha/beta T cells and gamma/delta T cells, B cells, natural killer (NK) cells, natural killer T (NKT) cells, mast cells, and myeloic-derived phagocytes. In some embodiments, the immune effector cell(s) is allogenic. In some embodiments, the immune effector cell(s) is autologous. Immune effector cells such as T cells may be activated and expanded generally using methods previously described, such as for example, as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, all of which are incorporated herein by reference in their entirety.

“Bi-specific chimeric antigen receptor” refers to a CAR that includes two domains, wherein the first domain is specific for a first ligand/antigen/target, and wherein the second domain is specific for a second ligand/antigen/target. In some forms, the ligand is a B-cell specific protein, a tumor-specific ligand/antigen/target, a tumor associated ligand/antigen/target, or combinations thereof. A bispecific CAR is specific to two different antigens. A multi-specific or multivalent CAR is specific to more than one different antigen, e.g., 2, 3, 4, 5, or more. In some forms, a multi-specific or multivalent CAR targets and/or binds three or more different antigens.

“Encoding” or “encode” refers to the property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “locus” is the specific physical location of a DNA sequence (e.g., of a gene) on a chromosome. It is understood that a locus of interest can not only qualify a nucleic acid sequence that exists in the main body of genetic material (i.e., in a chromosome) of a cell but also a portion of genetic material that can exist independently to said main body of genetic material such as plasmids, episomes, virus, transposons or in organelles such as mitochondria as non-limiting examples.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

In the context of cells, the term “isolated” also refers to a cell altered or removed from its natural state. That is, the cell is in an environment different from that in which the cell naturally occurs, e.g., separated from its natural milieu such as by concentrating to a concentration at which it is not found in nature. “Isolated cell” is meant to include cells that are within samples that are substantially enriched for the cell of interest and/or in which the cell of interest is partially or substantially purified.

As used herein, “transformed,” “transduced,” and “transfected” encompass the introduction of a nucleic acid or other material into a cell by one of a number of techniques known in the art.

A “vector” is a composition of matter which includes an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” encompasses an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus (AAV) vectors, retroviral vectors, and the like.

“Tumor burden” or “tumor load” as used herein, refers to the number of cancer cells, the size or mass of a tumor, or the total amount of tumor/cancer in a particular region of a subject. Methods of determining tumor burden for different contexts are known in the art, and the appropriate method can be selected by the skilled person. For example, in some forms, tumor burden can be assessed using guidelines provided in the Response Evaluation Criteria in Solid Tumors (RECIST).

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

The term “inhibit” or other forms of the word such as “inhibiting” or “inhibition” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with or restraining the expression and/or activity of the gene/gene product pathway relative to a standard or a control.

“Treatment” or “treating” means to administer a composition to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., cancer). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “functional fragment” or “functional variant” means a fragment or variant of a polypeptide, such as a full-length or native polypeptide, that retains one or more functional properties of the full-length or native polypeptide. For example, in some embodiments, a functional fragment or functional variant of an IDR polypeptide is a fragment or variant that retains the function of enhancing condensation of CAR as a CAR-IDR fusion polypeptide compared to a control CAR polypeptide without an IDR.

As used herein, the terms “variant” or “active variant” refers to a polypeptide or polynucleotide that differs from a reference polypeptide or polynucleotide, but retains one or more functional properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Len, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needleman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the disclosure.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. Compositions

It has been established that polypeptides derived from intrinsically disordered regions (IDRs) of proteins impart the function of enhanced receptor signaling at the cell surface. Compositions including isolated polypeptides of disordered regions of proteins (“IDR” polypeptides), and fusion proteins incorporating the IDR polypeptides together with one or more heterologous sequences are provided. Chimeric Antigen Receptors (CAR) including one, two, three or more IDR polypeptides are provided (i.e., CAR-IDR). In preferred embodiments, the CAR-IDR fusion peptides include at least one IDR polypeptide, for example, any of SEQ ID NOs:1-6. In further preferred embodiments, the CAR-IDR fusion peptides have amino acid sequence of any of SEQ ID NOs:7-27. Recombinant constructs including nucleic acids expressing or encoding the polypeptides and fusion proteins are also provided. Viral genomes including the recombinant constructs, recombinant viruses including the constructs, and vaccine formulations formed thereof are also provided.

The disclosed compositions and methods are especially applicable to development of enhanced chimeric antigen receptor engineered T cell therapy (CAR-T). Compositions of genetically modified T lymphocytes that have enhanced anti-tumor activity are described.

### A. Intrinsically Disordered Regions (IDRs)

Intrinsically disordered regions (IDRs) are commonly present in about 30% of the human proteome. They do not typically fold into a well-defined 3-dimensional structure. However, some of them self-assemble via weak inter and intra molecular interactions to form biomolecular condensates, which drive the formation of membranelles organelles in the intracellular environment. These condensates displayed unique biochemical activities through enriching and organizing effector molecules to promote signaling (Xiao, Q., McAtee, C. K. & Su, X. Nat Rev Immunol 22, 188-199; Case, L. B., Ditlev, J. A. & Rosen, M. K. Annu Rev Biophys 48, 465-494, doi:10.1146/annurev-biophys-052118-115534 (2019)).

Compositions including and encoding a polypeptide including a fragment of the intrinsically disordered region gene product or variant thereof, referred to herein as “IDR polypeptides”, are provided. Typically, the IDR polypeptide is between about 50 amino acids and about 500 amino acids, or any specific integer number of amino acids therebetween, including, but not limited to 60, 70, 80, 90, 100, 125, 150, 175, 200, 225, 250, 275, 300, 350, 400, or 450 amino acids; preferably, at least a length that is sufficient to promote the condensation of CAR and enhance T cell activation when forming a fusion protein with CAR. The IDR polypeptides, nucleic acids encoding the same, and delivery vehicles thereof, and cells including them can optionally include one or more additional heterologous proteins, polypeptides, or other amino acid sequences. Typically, the presence of one or more IDR polypeptides within the cytoplasmic domain of a recombinant fusion peptide will impart an increased cell activation and/or signaling to/from the fusion peptide. Thus, in some embodiments, IDR domains are described as part of a fusion protein.

Recognition of the natural abundance and functional importance of intrinsically disordered proteins (IDPs), and protein hybrids that contain both intrinsically disordered regions (IDRs) and ordered regions, is changing protein science. IDPs and IDRs, i.e., functional proteins and protein regions without unique structures, can often be found in all organisms, and typically play vital roles in various biological processes. Disorder-based functionality complements the functions of ordered proteins and domains. However, by virtue of their existence, IDPs/IDRs, which are characterized by remarkable conformational flexibility and structural plasticity, break multiple rules established over the years to explain structure, folding, and functionality of well-folded proteins with unique structures. Despite the general belief that unique biological functions of proteins require unique 3D-structures (which dominated protein science for more than a century), structure-less IDPs/IDRs are functional, being able to engage in biological activities and perform tricks that are highly unlikely for ordered proteins.

The development of tools for reliable prediction of intrinsic disorder predisposition based on sequence data has presented the opportunity to evaluate the commonness of IDPs/IDRs at the proteome level. Already, the first application of one of such tools to the Swiss Protein Database produced a very “big catch”: long IDPRs of at least 40 consecutive residues were predicted to be present in over 15,000 proteins, and particularly high disorder scores were found for more than 1,000 proteins (Romero P, et al., Pac Symp Biocomput. (1998) 1998:437-48). Analysis of proteins encoded by 31 genomes from 3 kingdoms of life showed that eukaryotes typically have a higher disorder score than either archaea or prokaryotes, since 52-67% of eukaryotic proteins have long IDPRs as compared to 26-51 and 16-45% proteins with such long IDPRs in archaea and bacteria, respectively (Dunker A K, et al., Genome Inform Ser Workshop Genome Inform. (2000) 11:161-71; Oldfield C J, et al., Biochemistry (2005) 44:1989-2000). The natural abundance of intrinsic disorder was later supported by multiple comprehensive computational studies (Walsh I, et al., Bioinformatics (2015) 31:201-8; Burra P V, et al., PLoS ONE (2010) 5:e12069).

On multiple levels, ranging from amino acid composition to amino acid sequence, to structural content and spatial organization, ordered proteins, and domains are obviously much more complex than IDPs and IDRs. It seems that this global, multi-level simplicity of IDPs/IDRs is rooted in the peculiarities of their amino acid sequences, which are known to be depleted in order-promoting residues (Trp, Cys, Tyr, Ile, Phe, Val, Asn, and Leu) and enriched in disorder-promoting residues (Arg, Pro, Gln, Gly, Glu, Ser, Ala, and Lys) and commonly contain repeats. Therefore, IDPs/IDRs are characterized by the reduced informational content of their amino acid sequences, and their amino acid alphabet is decreased in comparison with the alphabet utilized in the amino acid sequences of ordered domains and proteins (Uversky V N. Intrins Disord Proteins (2016) 4:e1135015). Furthermore, since they do not need to gain any sort of ordered structure, IDPs/IDRs have a noticeably greater sequence space than that of ordered proteins (Uversky V N. Biochim Biophys Acta (2013) 1834:932-51). Therefore, the sequence simplicity associated with intrinsic disorder is translated into an immensely expanded sequence space, which, in its turn, gives rise to the enormous structural complexity of these proteins and regions.

Similarly, despite their inability to fold into unique structures in isolation and despite their simplified spatial organization and globally reduced structural content, IDPs are characterized by exceptional spatiotemporal heterogeneity, where parts of an IDP are dis/ordered to varying degrees at one moment in time, but can change state at a future point in time (Uversky V N. Biochim Biophys Acta (2013) 1834:932-51; Uversky V N. Intrins Disord Proteins (2016) 4:e1135015). Therefore, IDPs are not homogeneous, but represent a very complex mixture of a broad variety of partially foldable, potentially foldable, differently foldable, or completely unfoldable segments. This behavior of an IDP as a highly frustrated system that does not possess a singular folded state is reflected in its free energy landscape, which is relatively flat and simple, lacks a deep energy minimum that can be found within ordered proteins, and appears as a “hilly plateau,” where the “hills” correspond to forbidden conformations (Fisher C K, Stultz C M. Curr Opin Struct Biol. (2011) 21:426-31; Turoverov K K, Kuznetsova I M, Uversky V N. Prog Biophys Mol Biol. (2010) 102:73-84; Uversky V N, Oldfield C J, Dunker A K. Annu Rev Biophys. (2008) 37:215-46). Such a simplified and flattened energy landscape is extremely sensitive to different environmental changes that can modify the landscape in several different ways, lowering some energy minima while raising some energy barriers. This explains the conformational plasticity of an IDP/IDR, its extreme sensitivity to changes in the environment, its ability to interact with multiple different partners, and consequently to fold in different ways. Therefore, this exceptional spatio-temporal heterogeneity of IDPs/IDRs, where functional elements have varying degrees of disorder and have unique responses to environmental changes, is directly related the astonishing multifunctionality of disordered proteins that are able to control, regulate, interact with, as well as be controlled and regulated by, a plethora of structurally unrelated partners. In summary, the multilevel structural, spatiotemporal, and sequential simplicity of IDPs/IDRs and their simplified energy landscapes define the structural heterogeneity and exceptional functional complexity of disordered proteins and regions.

Online tools can be used to identify regions of proteins that are intrinsically disordered. Exemplary online tools include IUPred2A. IUPred2A is a combined prediction tool designed to discover intrinsically disordered or conditionally disordered proteins and protein regions. Methods of analyzing protein disorder using IUPred2A has been previously described (Mészáros B et al., Nucleic Acids Research 2018; 46(W1):W329-W337; Erdös G and Dosztányi Z, Curr Protoc Bioinformatics. 2020 June; 70(1):e99). In some embodiments, suitable IDRs are first determined using IUPred2A prior to using functional assays to assess their ability to promote condensation or CAR, T cell activation, and/or tumor cell cytotoxicity as described in the Examples.

### 1. Exemplary IDRs and Variants

In some embodiments, the IDR is one derived from RNA-binding protein FUS. An exemplary amino acid sequence of IDR derived from FUS is:

(SEQ ID NO: 1)
MASNDYTQQATQSYGAYPTQPGQGYSQQSSQPYGQQSYSGYSQSTDTSGYG
QSSYSSYGQSQNTGYGTQSTPQGYGSTGGYGSSQSSQSSYGQQSSYPGYGQ
QPAPSSTSGSYGSSSQSSSYGQPQSGSYSQQPSYGGQQQSYGQQQSYNPPQ
GYGQQNQYNSSSGGGGGGGGGGNYGQDQSSMSSGGGSGGGYGNQDQSGGGG
SGGYGQQDRG

In some embodiments, the IDR is one derived from RNA-binding protein EWS. RNA-binding protein EWS is a protein that in humans is encoded by the EWSR1 gene on human chromosome 22, specifically 22q12.2. An exemplary amino acid sequence of IDR derived from EWS is:

(SEQ ID NO: 2)
GYAQTTQAYGQQSYGTYGQPTDVSYTQAQTTATYGQTAYATSYGQPPTGYT
TPTAPQAYSQPVQGYGTGAYDTTTATVTTTQASYAAQSAYGTQPAYPAYGQ
QPAATAPTRPQDGNKPTETSQPQSSTGGYNQPSLGYGQSNYSYPQVPGSYP
MQPVTAPPSYPPTSYSSTQPTSYDQSSYSQQNTYGQPSSYGQQSSYGQQSS
YGQQPPTSYPPQTGSYSQAPSQYSQQSSSYG

In some embodiments, the IDR is one derived from TATA box binding protein (TBP)-associated factor 15 (TAF15). An exemplary amino acid sequence of IDR derived from TAF15 is:

(SEQ ID NO: 3)
MSDSGSYGQSGGEQQSYSTYGNPGSQGYGQASQSYSGYGQTTDSSYGQNYS
GYSSYGQSYSQSYGGYENQKQSSYSQQPYNNQGQQQNMESSGSQGGRAPSY
DQPDYGQQDSYDQQSGYDQHQGSYDEQSNYDQQHDSYSQNQQSYHSQRENY
SHHTQDDRRDVSRYGEDNRGYGGSQGG

In some embodiments, the IDR is one derived from nuclear pore complex protein Nup98. An exemplary amino acid sequence of IDR derived from Nup98 is:

(SEQ ID NO: 4)
FNKSFGTPFGGGIGGFGTTSTFGQNTGFGTTSGGAFGTSAFGSSNNTGGL
FGNSQTKPGGLFGTSSFSQPATSTSTGFGFGTSTGTANTLFGTASTGTSLF
SSQNNAFAQNKPTGFGNFGTSTSSGGLFGTTNTTSNPFGSTSGSLFGPSSF
TAAPTGTTIKFNPPTGTDTMVKAGVSTNISTKHQCITAMKEYESKSLEELR
LEDYQANRKGPQNQVGAGTTTGLFGSSPATSSATGLFSSSTINSGFAYGQN
KTAFGTSTTGFGINPGGLFGQQNQQTTSLFSKPFGQATTTQNTGFSFGNTS
TIGQPSTNTMGLFGVTQASQPGGLFGTATNTSTGT

In some embodiments, the IDR is one derived from TAR DNA binding protein. An exemplary amino acid sequence of IDR derived from TDP43 is:

(SEQ ID NO: 5)
SNRQLERSGREGGNPGGFGNQGGFGNSRGGGAGLGNNQGSNMGGGMNFGAF
SINPAMMAAAQAALQSSWGMMGMLASQQNQSGPSGNNQNQGNMQREPNQAF
GSGNNSYSGSNSGAAIGWGSASNAGSGSGFNGGFGSSMDSKSSGWGM

In some embodiments, the IDR is a synthetic polypeptide having the desired functional properties. An exemplary amino acid sequence of a synthetic IDR is:

(SEQ ID NO: 6)
SKGPGRGDSPYSGRGDSPYSGRGDSPYSGRGDSPYSGRGDSPYSGRGDSPY
SGRGDSPYSGRGDSPYSGRGDSPYSGRGDSPYSGY

In some embodiments, the IDR is one, two, three, or more amino acid sequences of SEQ ID NOs:1-6, or one, two, three or more copies of one, two, three or more of SEQ ID NOs:1-6.

It has been discovered that the IDR polypeptide is sufficient to promote the condensation of CAR and enhance T cell activation of a recombinant fusion protein including the IDR polypeptide and one or more additional domains. Thus, compositions and methods of use of IDR peptide and fusion peptides thereof are provided. The compositions typically are, or include, an IDR polypeptide (e.g., SEQ ID NOs:1-6) or a functional fragment or variant thereof, or a nucleic acid encoding the same. Functional fragments and variants can be, for example, any number of amino acids sufficient to drive enhanced condensation of CAR and T cell activation. The data below supports the conclusions that IDR enhances tumor cell killing by CAR-T cells having a CAR-IDR fusion, by increased condensation of CAR and T cell activation.

In some embodiments, the IDR polypeptide is between about 50 amino acids and about 500 amino acids, or any specific integer number of amino acids therebetween, including, but not limited to 60, 70, 80, 90, 100, 125, 150, 175, 200, 225, 250, 275, 300, 350, 400, or 450 amino acids; preferably, at least a length that is sufficient to promote the condensation of CAR and enhance T cell activation. Variants can have, for example, at least 65%, 70%, 75%, 80%, 85%, 90%, 95%, 99% or 100% sequence identity to any one of SEQ ID NOs:1-6, or a functional fragment thereof; or the corresponding sequence of a homologue such as an orthologue or paralogue of any of the foregoing sequences; or any combination thereof. In a particular embodiment, a variant IDR polypeptide has at least 75%, 80%, 85%, 90%, 95%, 99% or 100% sequence identity to SEQ ID NO:1. In a particular embodiment, a variant IDR polypeptide has at least 75%, 80%, 85%, 90%, 95%, 99% or 100% sequence identity to SEQ ID NO:2. In a particular embodiment, a variant IDR polypeptide has at least 75%, 80%, 85%, 90%, 95%, 99% or 100% sequence identity to SEQ ID NO:3. In a particular embodiment, a variant IDR polypeptide has at least 75%, 80%, 85%, 90%, 95%, 99% or 100% sequence identity to SEQ ID NO:4. In a particular embodiment, a variant IDR polypeptide has at least 75%, 80%, 85%, 90%, 95%, 99% or 100% sequence identity to SEQ ID NO:5. In a particular embodiment, a variant IDR polypeptide has at least 75%, 80%, 85%, 90%, 95%, 99% or 100% sequence identity to SEQ ID NO:6. Preferably variants maintain the ability to promote the condensation of CAR and enhance T cell activation. In some embodiments, an IDR polypeptide variant is considered to be “functional” if it maintains the ability to promote the condensation of CAR and enhance T cell activation. In some embodiments, IDR polypeptide variants are identified as functional if they enhance cytotoxicity in vitro or in vivo towards target cells.

Any of the IDR polypeptide sequences including the amino acid sequences of SEQ ID NOs:1-6 can include one or more amino acid substitutions. Typically, the amino acid substitutions do not impact the ability of the IDR domain of a fusion peptide to promote cell activation and/or signaling, for example in the case of CAR-IDR fusion proteins. Amino acid substitutions within CT peptides are generally based on the relative similarity of the amino acid side-chain substituents, for example, their hydrophobicity, hydrophilicity, charge, size, and the like. Exemplary substitutions of amino acids within any of SEQ ID NOs:1-6 can include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of an IDR polypeptide, as set forth in any one of SEQ ID NOs:1-6. In particular embodiments, the IDR polypeptides can include variants having about 50%, 60%, 70%, 80%, 90%, and 95% sequence identity to the polypeptide of SEQ ID NOs:1-6. Such variants and fragments can be used alone or in combination with each other.

In preferred embodiments, suitable IDRs are those sufficient to induce biomolecular condensates inside a eukaryotic cell or on the cell surface.

### B. IDR Fusion Peptides

IDR fusion proteins, including one or more heterologous polypeptide sequences fused to one or more IDR polypeptides are provided.

The term “IDR domain” is used exclusively in the context of fusion peptides that include one or more copies of one or more IDR polypeptides, to refer to the component of the fusion peptide that includes the IDR polypeptide(s).

The term “IDR fusion protein” or “IDR fusion peptide” or “IDR fusion” refers to a polypeptide that includes an IDR domain and one or more heterologous amino acid sequences. A heterologous sequence for inclusion within an IDR fusion protein is not a component of an IDR peptide.

The term “Fusion peptide domain” refers to a heterologous sequence that is directly or indirectly fused to an IDR domain. Typically, a fusion peptide domain includes at least one amino acid that is on the extracellular side of the cell membrane, i.e., constitutes an extracellular domain. In some embodiments, the fusion peptide domain also includes a transmembrane domain.

In some embodiments, IDR-fusion proteins are engineered to include one or more IDR domains as a cytosolic component of the IDR fusion protein. In preferred embodiments, IDR fusion peptides include one or more heterologous polypeptides fused to an intracellular component, including an IDR domain that includes one or more of SEQ ID NOs:1-6, or a functional homologue or variant thereof. In other embodiments, IDR-fusion proteins are engineered to include one or more IDR domains as an extracellular component of the IDR fusion protein.

An exemplary heterologous polypeptide for inclusion within IDR-fusion peptides includes the ectodomain and transmembrane domain of a cell-surface protein, such as an ectodomain of a cell-surface receptor that is not a component of the protein where the IDR is derived from.

In some embodiments, IDR-fusion proteins include one or more extracellular polypeptide domains, and one or more transmembrane domains, and optionally an intracellular domain, where the transmembrane domain or optionally the intracellular domain is fused to the IDR domain(s). In some embodiments, where a fusion protein includes one or more intracellular domains, the one or more IDR domains are fused to the amino (N) or carboxyl (C) terminus of the intracellular domains. In some embodiments, the fusion proteins include an entire endogenous protein fused to one or more IDR domains.

### 1. Heterologous Sequences

Heterologous elements that can be associated with, linked, conjugated, or otherwise attached directly or indirectly to the IDR polypeptide sequence(s), or nucleic acids expressing the IDR polypeptides are disclosed. Such molecules include, but are not limited to, protein domains, such as transduction domains, fusogenic peptides, targeting molecules, and sequences that enhance protein expression and/or isolation. Suitable protein domains include ectodomains, transmembrane domains, cytoplasmic domains of proteins and macromolecular structures including combinations of ectodomains, transmembrane domains, and cytoplasmic domains. Typically, the other protein domains are not proteins from which the IDR domain is derived. In some embodiments, the other protein domains have or have potential for one or more molecular functions or activities. Such “functional” domains can be engineered to provide one or more functions or activities, as desired. Exemplary functions include receptor or ligand binding, enzymic activity, and molecular transport, such as active transport of one or more molecules into or out of one or more cellular compartments. In some embodiments, the other protein domains within an IDR fusion protein bind to a specific substrate or molecule. An exemplary molecule is an antigen or a cell-surface receptor.

Thus, in some embodiments, IDR fusion peptides include one or more heterologous peptide domains, such as receptors at the surface of a cell, optionally including a transmembrane domain that anchors or connects the ectodomain to the cell surface and connects with the intracellular IDR domain.

Exemplary cell surface receptors coordinate the activity of cells upon interaction with other cells, such as immune cells, such as T cells. For example, in some embodiments, the heterologous domain is a recombinant or engineered chimeric antigen receptor (CAR). In other embodiments, the heterologous domain includes a specific transmembrane domain (e.g., transmembrane domain of CD8 or CD28) for further enhancing signaling and receptor sensitivity in the case of CAR. In further embodiments, the heterologous domain is a co-signaling domain. Exemplary co-signaling domains have been previously described (Majzner, R. G. et al., Cancer Discov, doi:10.1158/2159-8290. CD-19-0945 (2020); Heitzeneder, S. et al., Cancer Cell 40, 53-69 e59, (2022); Priceman, S. J. et al., Oncoimmunology 7, e1380764, (2018)) In some embodiments, the fusion peptides include multiple heterologous domains, such as a CAR domain (e.g., a single chain variable fragment (scFv, FMC63) targeting CD19), a transmembrane domain of CD8a, cytosolic signaling domains or a fragment there of derived from CD28, 41BB, and CD3Q.

a. Chimeric Antigen Receptors (CAR)

In some forms, the fusion protein includes a Chimeric Antigen Receptor (CAR) fused with one or more IDR domains (“CAR-IDR”). Typically, CARs include a transmembrane domain and one or more intracellular/cytoplasmic domains.

In the first generation of CARs, there was only one intracellular signal component (e.g., CD3 zeta). The second generation of CAR added one costimulatory molecule on the basis of the first generation (e.g., CD28, 4-1BB). Based on the second generation of CARs, the third generation of CAR added another costimulatory molecule. Fourth-generation of CAR T cells can activate the downstream transcription factor to induce cytokine production after the CAR recognizes the target antigens. The fifth-generation of CARs, based on the second generation, uses gene editing to inactivate the TRAC gene, leading to the removal of the TCR alpha and beta chains. See, e.g., Zhao, Lijun & Cao, Yu. (2019). Engineered T Cell Therapy for Cancer in the Clinic. Frontiers in Immunology. 10.10.3389/fimmu.2019.02250. Any of these CAR strategies and structures can serve as a starting point for the addition of one or more IDRs.

Thus, CAR fusion proteins are modular in structure, containing several discrete domains (e.g., additional examples discussed below). The IDR can be added or inserted into a CAR fusion protein at any location (e.g., between, before, or after existing domains) provided the CAR remains functional (e.g., the activity in antigen binding and in activating T cells). For example, the IDR can be inserted/added before or after the scFv or equivalent domains that bind antigens, or any intracellular signaling domains (e.g., co-stimulatory domain(s)) of the CAR fusion protein.

Therefore, in some embodiments, a CAR-IDR fusion protein includes an entire CAR with one or more IDR domains fused at the C terminus. In some embodiments, the CAR includes an intracellular domain that includes a component of endogenous CD28, 41BB, and/or CD3 zeta protein. Therefore, in some embodiments, a CAR-IDR fusion protein includes an entire CAR, with one or more IDR domains fused directly to the end of the CD3 zeta region, such that both the CD3 zeta and contiguous IDT domain(s) are expressed in the cytoplasmic compartment. Additionally or alternatively, IDR(s) can be fused before or after (e.g., N-terminal or C-terminal to) the antigen binding domain, transmembrane domain, CD28, 41BB, CD3 zeta protein, or other intracellular or co-stimulatory domains.

i. CAR Structure

CARs are engineered receptors that possess both antigen-binding and T-cell-activating functions. Immunotherapy using T cells genetically engineered to express a CAR is rapidly emerging as a superior new treatment for hematological and non-hematological malignancies. Based on the location of the CAR in the membrane of the cell, the CAR can be divided into three main distinct domains, including an extracellular antigen-binding domain, followed by a space region, a transmembrane domain, and the intracellular signaling domain. The antigen-binding domain, most commonly derived from variable regions of immunoglobulins, typically contains VH and VL chains that are joined up by a linker to form the so-called “scFv.” The segment interposing between the antigen-binding domain (e.g., scFv) and the transmembrane domain is a “spacer domain.” The spacer domain can include the constant IgGI hinge-CH2-CH3 Fc domain. In some cases, the spacer domain and the transmembrane domain are derived from CD8. The intracellular signaling domains mediating T cell activation can include a CD3ζ co-receptor signaling domain derived from C-region of the TCR α and β chains and one or more costimulatory domains. In some forms, conjugation with one or more IDR domains includes addition of the one or more IDR domains immediately next to the costimulatory domains of the CAR.

In some forms, the antigen-binding domain is derived from an antibody. The term antibody herein refers to natural or synthetic polypeptides that bind a target antigen. The term includes polyclonal and monoclonal antibodies, including intact antibodies and functional (e.g., antigen-binding) antibody fragments, including Fab fragments, F(ab′)₂ fragments, Fab′ fragments, Fv fragments, recombinant IgG (rlgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. The term also encompasses intact or full-length antibodies, including antibodies of any class or subclass, including IgG and sub-classes thereof, IgM, IgE, IgA, and IgD. The antigen-binding domain of a CAR can contain complementary determining regions (CDR) of an antibody, variable regions of an antibody, and/or antigen binding fragments thereof. For example, the antigen-binding domain for a CD19 CAR can be derived from a human monoclonal antibody to CD19, such as those described in U.S. Pat. No. 7,109,304, which is specifically incorporated by reference herein in its entirety for use in accordance with the disclosed compositions and methods. In some forms, the antigen-binding domain can include an F(ab′)2, Fab′, Fab, Fv or scFv.

In some forms, the CAR includes one or more spacer domain(s) (also referred to as hinge domain) that is located between the extracellular antigen-binding domain and the transmembrane domain. A spacer domain is an amino acid segment that is generally found between two domains of a protein and may allow for flexibility of the protein and movement of one or both of the domains relative to one another. Any amino acid sequence that provides such flexibility and movement of the extracellular antigen-binding domain relative to the transmembrane domain can be used. The spacer domain can be a spacer or hinge domain of a naturally occurring protein. In some forms, the hinge domain is derived from CD8a, such as, a portion of the hinge domain of CD8a, e.g., a fragment containing at least 5 (e.g., 5, 10, 15, 20, 25, 30, 35, or 40) consecutive amino acids of the hinge domain of CD8a. Hinge domains of antibodies, such as an IgG, IgA, IgM, IgE, or IgD antibodies can also be used. In some forms, the hinge domain is the hinge domain that joins the constant CH1 and CH2 domains of an antibody. Non-naturally occurring peptides may also be used as spacer domains. For example, the spacer domain can be a peptide linker, such as a (G×S)n linker, wherein x and n, independently can be an integer of 3 or more, including 3, 4, 5, 6, 7, 8, 9, 10, 11, 12, or more.

In some forms, the CAR includes a transmembrane domain that can be directly or indirectly fused to the antigen-binding domain. The transmembrane domain may be derived either from a natural or a synthetic source. In some forms, the transmembrane domain of the CAR includes a transmembrane domain of an alpha, beta, or zeta chain of a T-cell receptor, CD8, CD4, CD28, CD137, CD80, CD86, CD152 (CTLA-4) or PD1, or a portion thereof. Transmembrane domains can also contain at least a portion of a synthetic, non-naturally occurring protein segment. In some forms, the transmembrane domain is a synthetic, non-naturally occurring alpha helix or beta sheet. In some forms, the protein segment is at least about 15 amino acids, e.g., at least 15, 16, 17, 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776.

The intracellular signaling domain is responsible for activation of at least one of the normal effector functions of the immune effector cell expressing the CAR. The term effector function refers to a specialized function of a cell. Effector function of a T cell, for example, may be cytolytic activity or helper activity including the secretion of cytokines. In some forms, an intracellular signaling domain includes the zeta chain of the T cell receptor or any of its homologs (e.g., eta, delta, gamma, or epsilon), MBl chain, B29, Fc RIII, Fc RI and combinations of signaling molecules such as CD3ζ and CD28, 4-1BB, OX40 and combination thereof, as well as other similar molecules and fragments. Intracellular signaling portions of other members of the families of activating proteins can be used, such as FcγRIII and FcεRI.

Many immune effector cells require co-stimulation, in addition to stimulation of an antigen-specific signal, to promote cell proliferation, differentiation and survival, as well as to activate effector functions of the cell. Therefore, in some forms, the CAR includes at least one co-stimulatory signaling domain. The term co-stimulatory signaling domain, refers to at least a portion of a protein that mediates signal transduction within a cell to induce an immune response such as an effector function. The co-stimulatory signaling domain can be a cytoplasmic signaling domain from a co-stimulatory protein, which transduces a signal and modulates responses mediated by immune cells, such as T cells, NK cells, macrophages, neutrophils, or eosinophils. In some forms, the co-stimulatory signaling domain is derived from a co-stimulatory molecule selected from CD27, CD28, CD137, 0X40, CD30, CD40, CD3, LFA-1, ICOS, CD2, CD7, LIGHT, NKG2C, B7-H3, ligands of CD83 and combinations thereof. In one form, the co-stimulatory signaling domain is derived from CD28.

CARs can be used in order to generate immuno-responsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and PCT Publication WO 9215322, each of which is specifically incorporated by reference herein in its entirety). Alternative CAR constructs can be characterized as belonging to successive generations. First-generation CARs typically include a single-chain variable fragment of an antibody specific for an antigen, for example including a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936, each of which is specifically incorporated by reference herein in its entirety). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3C; see U.S. Pat. Nos. 8,911,993; 8,916,381; 8,975,071; 9,101,584; 9,102,760; 9,102,761, each of which is specifically incorporated by reference herein in its entirety). Third-generation CARs include a combination of costimulatory endodomains, such a CD3ζ-chain, CD97, GDI1a-CD18, CD2, ICOS, CD27, CD154, CDS, OX40, 4-1BB, or CD28 signaling domains (for example scFv-CD28-4-1BB-CD3ζ or scFv-CD28-OX40-CD3C; see U.S. Pat. Nos. 8,906,682; 8,399,645; 5,686,281; PCT Publication No. WO2014134165; PCT Publication No. WO2012079000, each of which is specifically incorporated by reference herein in its entirety). Alternatively, co-stimulation can be orchestrated by expressing CARs in antigen-specific T cells, chosen so as to be activated and expanded following engagement of their native αβTCR, for example by antigen on professional antigen-presenting cells, with attendant co-stimulation. Any of the first, second, or third generation CARs described above can be used in accordance with the disclosed compositions and methods.

ii. Exemplary Antigens and CAR Embodiments

The target specificity of the cell expressing a CAR is determined by the antigen recognized by the antibody/ectodomain. The disclosed compositions and methods can be used to create constructs, and cells expressing the constructs, that target any antigen. In the context of immunotherapy, particularly cancer immunotherapy, numerous antigens, and suitable ectodomains for targeting them, are well known. Unlike the native TCR, the majority of scFv-based CARs recognize target antigens expressed on the cell surface rather than internal antigens that are processed and presented by the cells' MHC, however, CARs have the advantage over the classical TCR that they can recognize structures other than protein epitopes, including carbohydrates and glycolipids Dotti, et al., Immunol Rev. 2014 January; 257(1):. doi:10.1111/imr.12131 (35 pages) thus increasing the pool of potential target antigens. Preferred targets include antigens that are only expressed on cancer cells or their surrounding stroma (Cheever, et al., Clin Cancer Res., 15:5323-5337 (2009)), such as the splice variant of EGFR (EGFRvI1I), which is specific to glioma cells (Sampson, et al., Semin Immunol., 20(5):267-75 (2008)). However, human antigens meet this requirement, and the majority of target antigens are expressed either at low levels on normal cells (e.g. GD2, CAIX, HER2) and/or in a lineage restricted fashion (e.g. CD19, CD20).

Preferred targets, and CARs that target them are known in the art (see, e.g., Dotti, et al., Immmunol Rev. 2014 January; 257(1):. doi:10.1111/imr.12131 (35 pages). For example, CAR targets for hematological malignancies include, but are not limited to, CD 19 (e.g., B-cell) (Savoldo, et al., J Clin Invest., 121:1822-1826 (2011), Cooper, et al., Blood, 105:1622-1631 (2005); Jensen, et al., Biol Blood Marrow Transplant (2010), Kochenderfer, et al., Blood, 119:2709-2720 (2012), Brentjens, et al., Molecular Therapy, 17:S157 (2009), Brentjens, et al., Nat Med., 9:279-286 (2003), Brentjens, et al., Blood, 118:4817-4828 (2011), Porter, et al., N Engl J Med., 365:725-733 (2011), Kalos, et al., Sci Transl Med., 3:95ra73 (2011), Brentjens, et al., Sci Transl Med., 5:177ra38 (2013), Grupp, et al., N Engl J Med (2013)); CD20 (e.g., B-cell) (Jensen, et al., Biol Blood Marrow Transplant (2010), Till, et al., Blood, 112:2261-2271 (2008), Wang, et al., Hum Gene Ther., 18:712-725 (2007), Wang, et al., Mol Ther., 9:577-586 (2004), Jensen, et al., Biol Blood Marrow Transplant, 4:75-83 (1998)); CD22 (e.g., B-cell) (Haso, et al., Blood, 121:1165-1174 (2013)); CD30 (e.g., B-cell) (Di Stasi, et al., Blood, 113:6392-6402 (2009), Savoldo, et al., Blood, 110:2620-2630 (2007), Hombach, et al., Cancer Res., 58:1116-1119 (1998)); CD33 (e.g., Myeloid) (Finney, et al., J Immunol., 161:2791-2797 (1998)); CD70 (e.g., B-cell/T-cell) (Shaffer, et al., Blood, 117:4304-4314 (2011)); CD123 (e.g., Myeloid) (Tettamanti, et al., Br J Haematol., 161:389-401 (2013)); Kappa (e.g., B-cell) (Vera, et al., Blood, 108:3890-3897 (2006)); Lewis Y (e.g., Myeloid) (Peinert, et al., Gene Ther., 17:678-686 (2010), Ritchie, et al., Mol Ther. (2013)); NKG2D ligands (e.g., Myeloid) (Barber, et al., Exp Hematol., 36:1318-1328 (2008), Lehner, et al., PLoS One., 7:e31210 (2012), Song, et al., Hum Gene Ther., 24:295-305 (2013), Spear, et al., J Immunol. 188:6389-6398 (2012)); ROR1 (e.g., B-cell) (Hudecek, et al., Clin Cancer Res. (2013)).

CAR targets for solid tumors include, but are not limited to, B7H3 (e.g., sarcoma, glioma) (Cheung, et al., Hybrid Hybridomics, 22:209-218 (2003)); CAIX (e.g., kidney) (Lamers, et al., J Clin Oncol., 24:e20-e22. (2006)), Weijtens, et al., Int J Cancer, 77:181-187 (1998)); CD44 v6/v7 (e.g., cervical) (Hekele, et al., Int J Cancer, 68:232-238 (1996)), Dall, et al., Cancer Immunol Immunother, 54:51-60 (2005); CD171 (e.g., neuroblastoma) (Park, et al., Mol Ther., 15:825-833 (2007)); CEA (e.g., colon) (Nolan, et al., Clin Cancer Res., 5:3928-3941 (1999)); EGFRvII (e.g., glioma) (Bullain, et al., J Neurooncol. (2009), Morgan, et al., Hum Gene Ther., 23:1043-1053 (2012)); EGP2 (e.g., carcinomas) (Meier, et al., Magn Reson Med., 65:756-763 (2011), Ren-Heidenreich, et al., Cancer Immunol Immunother., 51:417-423 (2002)); EGP40 (e.g., colon) (Daly, et al., Cancer Gene Ther., 7:284-291 (2000); EphA2 (e.g., glioma, lung) (Chow, et al., Mol Ther., 21:629-637 (2013)); ErbB2(HER2) (e.g., breast, lung, prostate, glioma) (Zhao, et al., J Immunol., 183:5563-5574 (2009), Morgan, et al., Mol Ther., 18:843-851 (2010), Pinthus, et al., 114:1774-1781 (2004), Teng, et al., Hum Gene Ther., 15:699-708 (2004), Stancovski, et al., J Immunol., 151:6577-6582 (1993), Ahmed, et al., Mol Ther., 17:1779-1787 (2009), Ahmed, et al., Clin Cancer Res., 16:474-485 (2010), Moritz, et al., Proc Nat Acad Sci U.S.A., 91:4318-4322 (1994)); ErbB receptor family (e.g., breast, lung, prostate, glioma) (Davies, et al., Mol Med., 18:565-576 (2012)); ErbB3/4 (e.g., breast, ovarian) (Muniappan, et al., Cancer Gene Ther., 7:128-134 (2000), Altenschmidt, et al., Clin Cancer Res., 2:1001-1008 (1996)); HLA-A1/MAGE1 (e.g., melanoma) (Willemsen, et al., Gene Ther., 8:1601-1608 (2001), Willemsen, et al., J Immunol., 174:7853-7858 (2005)); HLA-A2/NY-ESO-1 (e.g., sarcoma, melanoma) (Schuberth, et al., Gene Ther., 20:386-395 (2013)); FR-α (e.g., ovarian) (Hwu, et al., J Exp Med., 178:361-366 (1993), Kershaw, et al., Nat Biotechnol., 20:1221-1227 (2002), Kershaw, et al., Clin Cancer Res., 12:6106-6115 (2006), Hwu, et al., Cancer Res., 55:3369-3373 (1995)); FAP (e.g., cancer associated fibroblasts) (Kakarla, et al., Mol Ther. (2013)); FAR (e.g., rhabdomyosarcoma) (Gattenlohner, et al., Cancer Res., 66:24-28 (2006)); GD2 (e.g., neuroblastoma, sarcoma, melanoma) (Pule, et al., Nat Med., 14:1264-1270 (2008), Louis, et al., Blood, 118:6050-6056 (2011), Rossig, et al., Int J Cancer., 94:228-236 (2001)); GD3 (e.g., melanoma, lung cancer) (Yun, et al., Neoplasia., 2:449-459 (2000)); HMW-MAA (e.g., melanoma) (Burns, et al., Cancer Res., 70:3027-3033 (2010)); IL11Rα (e.g., osteosarcoma) (Huang, et al., Cancer Res., 72:271-281 (2012)); IL13Rα2 (e.g., glioma) (Kahlon, et al., Cancer Res., 64:9160-9166 (2004), Brown, et al., Clin Cancer Res. (2012), Kong, et al., Clin Cancer Res., 18:5949-5960 (2012), Yaghoubi, et al., Nat Clin Pract Oncol., 6:53-58 (2009)); Lewis Y (e.g., breast/ovarian/pancreatic) (Peinert, et al., Gene Ther., 17:678-686 (2010), Westwood, et al., Proc Natl Acad Sci U.S.A., 102:19051-19056 (2005), Mezzanzanica, et al., Cancer Gene Tlier., 5:401-407 (1998)); Mesothelin (e.g., mesothelioma, breast, pancreas) (Lanitis, et al., Mol Ther., 20:633-643 (2012), Moon, et al., Clin Cancer Res., 17:4719-4730 (2011)); Muel (e.g., ovarian, breast, prostate) (Wilkie, et al., J Immunol., 180:4901-4909 (2008)); NCAM (e.g., neuroblastoma, colorectal) (Gilham, et al., J Immunother., 25:139-151 (2002)); NKG2D ligands (e.g., ovarian, sacoma) (Barber, et al., Exp Hematol., 36:1318-1328 (2008), Lehner, et al., PLoS One, 7:e31210 (2012), Song, et al., Gene Ther., 24:295-305 (2013), Spear, et al., J Immunol., 188:6389-6398 (2012)); PSCA (e.g., prostate, pancreatic) (Morgenroth, et al., Prostate, 67:1121-1131 (2007), Katari, et al., HPB, 13:643-650 (2011)); PSMA (e.g., prostate) (Maher, et al., Nat Biotechnol., 20:70-75 (2002), Gong, et al., Neoplasia., 1:123-127 (1999)); TAG72 (e.g., colon) (Hombach, et al., Gastroenterology, 113:1163-1170 (1997), McGuinness, et al., Hum Gene Ther., 10:165-173 (1999)); VEGFR-2 (e.g., tumor vasculature) (J Clin Invest., 120:3953-3968 (2010), Niederman, et al., Proc Natl Acad Sci U.S.A., 99:7009-7014 (2002)).

In some forms, the CAR targets one or more antigens specific for cancer, an inflammatory disease, a neuronal disorder, HIV/AIDS, diabetes, a cardiovascular disease, an infectious disease, an autoimmune disease, or combinations thereof. One of skill in the art, based on general knowledge in the field and/or routine experimentation would be able to determine the appropriate antigen to be targeted by a CAR for a specific disease, disorder, or condition.

Exemplary antigens specific for cancer that could be targeted by the CAR include, but are not limited to, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGl, Ll-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, and combinations thereof.

In preferred forms, the CAR targets CD19, CD22, HER2 or any combinations thereof.

The results presented below show that coiled-coiled domains can reduce membrane localization of and signaling from CAR fusion proteins. Thus, in some embodiments, the IDR fusion proteins, particularly the IDR-CAR fusion proteins, lack coiled-coiled domain(s).

b. Exemplary Chimeric Antigen Receptors Including IDR Domains (CAR-IDR)

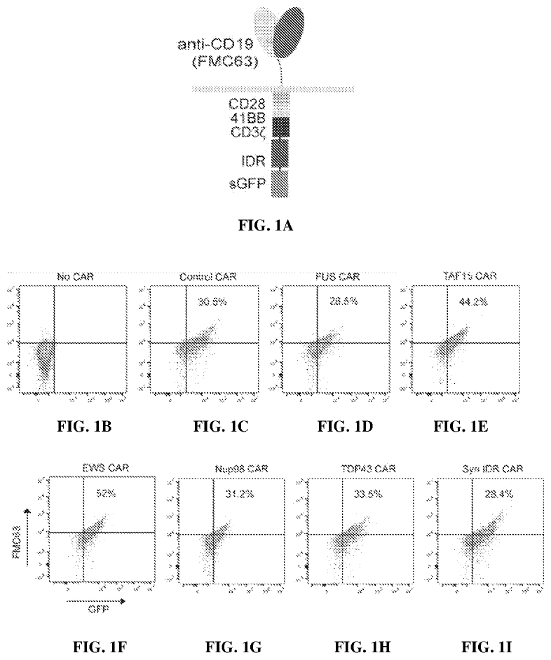

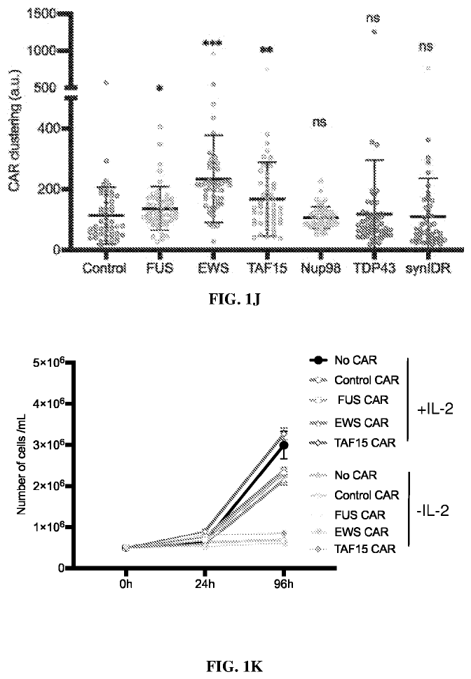

In some forms, the fusion peptide is a CAR including an IDR polypeptide conjugated to the carboxyl terminus of the CAR. An exemplary schematic for conjugation of a functional CAR with an IDR domain is provided in FIGS. 1A, 3A, and 4A.

In exemplary embodiments, the structure of a CAR-IDR is a fusion protein having the following features:

N-[CAR]-[IDR]_{Z}-C, where “N” and “C” refer to the amino (NH₂) and Carboxyl (COOH) termini, respectively, “CAR” refers to a single contiguous CAR fusion protein, “IDR” refers to an IDR and “Z” is an integer between one and four, inclusive, and. Preferably, Z is 1 or 2.

In some embodiments, the structure of a CAR-IDR is a fusion protein having the following features: N-[CAR part 1]-[IDR]_{Z}-[CAR part 2], where “N” and “C” refer to the amino (NH₂) and Carboxyl (COOH) termini, respectively, “IDR” refers to an IDR and “Z” is an integer between one and four, inclusive, “CAR-part 1” includes one or more traditional CAR domains and “CAR-part 2” includes one or more traditional CAR domains such that without the intervening [IDR]_{Z} domain, CAR-part 1 and CAR-part 2 form a single contiguous CAR fusion protein. Preferably, Z is 1 or 2.

In some embodiments, a CAR-IDR including one IDR domain fused to the cytoplasmic domain of the functional CAR includes the amino acid sequence of any of SEQ ID NOs:1-6 fused to the last residue of the CAR.

In some embodiments, the fusion peptide is a CAR including one or more IDR domains. An exemplary CAR-IDR is an anti-CD19 CAR-IDR. An exemplary amino acid sequence anti-CD19 CAR-IDR including an IDR derived from FUS is:

(SEQ ID NO: 7)
MALPVTALLLPLALLLHAARPDIQMTQTTSSLSASLGDRVTISCRASQDIS
KYLNWYQQKPDGTVKLLIYHTSRLHSGVPSRFSGSGSGTDYSLTISNLEQE
DIATYFCQQGNTLPYTFGGGTKLEITGGGGSGGGGSGGGGSEVKLQESGPG
LVAPSQSLSVTCTVSGVSLPDYGVSWIRQPPRKGLEWLGVIWGSETTYYNS
ALKSRLTIIKDNSKSQVFLKMNSLQTDDTAIYYCAKHYYYGGSYAMDYWGQ
GTSVTVSSGSGSEQKLISEEDLGSTTTPAPRPPTPAPTIASQPLSLRPEAC
RPAAGGAVHTRGLDFACDIYIWAPLAGTCGVLLLSLVITLYCSRSKRSRLL
HSDYMNMTPRRPGPTRKHYQPYAPPRDFAAYRSKRGRKKLLYIFKQPFMRP
VQTTQEEDGCSCRFPEEEEGGCELRVKFSRSADAPAYQQGQNQLYNELNLG
RREEYDVLDKRRGRDPEMGGKPQRRKNPQEGLYNELQKDKMAEAYSEIGMK
GERRRGKGHDGLYQGLSTATKDTYDALHMQALPPRTSGSGSGSGSMASNDY
TQQATQSYGAYPTQPGQGYSQQSSQPYGQQSYSGYSQSTDTSGYGQSSYSS
YGQSQNTGYGTQSTPQGYGSTGGYGSSQSSQSSYGQQSSYPGYGQQPAPSS
TSGSYGSSSQSSSYGQPQSGSYSQQPSYGGQQQSYGQQQSYNPPQGYGQQN
QYNSSSGGGGGGGGGGNYGQDQSSMSSGGGSGGGYGNQDQSGGGGSGGYGQ
QDRG

In some embodiments, the CAR-IDR is further conjugated to one or more fluorescent tags such as GFP. An exemplary amino acid sequence of an anti-CD19 CAR-IDR including an IDR derived from FUS and conjugated thereto a GFP is:

(SEQ ID NO: 8)
MALPVTALLLPLALLLHAARPDIQMTQTTSSLSASLGDRVTISCRASQDIS
KYLNWYQQKPDGTVKLLIYHTSRLHSGVPSRFSGSGSGTDYSLTISNLEQE
DIATYFCQQGNTLPYTFGGGTKLEITGGGGSGGGGSGGGGSEVKLQESGPG
LVAPSQSLSVTCTVSGVSLPDYGVSWIRQPPRKGLEWLGVIWGSETTYYNS
ALKSRLTIIKDNSKSQVFLKMNSLQTDDTAIYYCAKHYYYGGSYAMDYWGQ
GTSVTVSSGSGSEQKLISEEDLGSTTTPAPRPPTPAPTIASQPLSLRPEAC
RPAAGGAVHTRGLDFACDIYIWAPLAGTCGVLLLSLVITLYCSRSKRSRLL
HSDYMNMTPRRPGPTRKHYQPYAPPRDFAAYRSKRGRKKLLYIFKQPFMRP
VQTTQEEDGCSCRFPEEEEGGCELRVKFSRSADAPAYQQGQNQLYNELNLG
RREEYDVLDKRRGRDPEMGGKPQRRKNPQEGLYNELQKDKMAEAYSEIGMK
GERRRGKGHDGLYQGLSTATKDTYDALHMQALPPRTSGSGSGSGSMASNDY
TQQATQSYGAYPTQPGQGYSQQSSQPYGQQSYSGYSQSTDTSGYGQSSYSS
YGQSQNTGYGTQSTPQGYGSTGGYGSSQSSQSSYGQQSSYPGYGQQPAPSS
TSGSYGSSSQSSSYGQPQSGSYSQQPSYGGQQQSYGQQQSYNPPQGYGQQN
QYNSSSGGGGGGGGGGNYGQDQSSMSSGGGSGGGYGNQDQSGGGGSGGYGQ
QDRGGSGSMSKGEELFTGVVPILVELDGDVNGHKFSVRGEGEGDATNGKLT
LKFICTTGKLPVPWPTLVTTLTYGVQCFSRYPDHMKRHDFFKSAMPEGYVQ
ERTISFKDDGTYKTRAEVKFEGDILVNRIELKG
IDFKEDGNILGHKLEYNFNSHNVYITADKQKNGIKANFKIRHNVEDGSVQL
ADHYQQNTPIGDGPVLLPDNHYLSTQSVLSKDPNEKRDHMVLLEFVTAAGI
THGMDELYK

Another exemplary amino acid sequence anti-CD19 CAR-IDR including an IDR derived from TAF15 is:

(SEQ ID NO: 9)
MALPVTALLLPLALLLHAARPDIQMTQTTSSLSASLGDRVTISCRASQDIS
KYLNWYQQKPDGTVKLLIYHTSRLHSGVPSRFSGSGSGTDYSLTISNLEQE
DIATYFCQQGNTLPYTFGGGTKLEITGGGGSGGGGSGGGGSEVKLQESGPG
LVAPSQSLSVTCTVSGVSLPDYGVSWIRQPPRKGLEWLGVIWGSETTYYNS
ALKSRLTIIKDNSKSQVFLKMNSLQTDDTAIYYCAKHYYYGGSYAMDYWGQ
GTSVTVSSGSGSEQKLISEEDLGSTTTPAPRPPTPAPTIASQPLSLRPEAC
RPAAGGAVHTRGLDFACDIYIWAPLAGTCGVLLLSLVITLYCSRSKRSRLL
HSDYMNMTPRRPGPTRKHYQPYAPPRDFAAYRSKRGRKKLLYIFKQPFMRP
VQTTQEEDGCSCRFPEEEEGGCELRVKFSRSADAPAYQQGQNQLYNELNLG
RREEYDVLDKRRGRDPEMGGKPQRRKNPQEGLYNELQKDKMAEAYSEIGMK
GERRRGKGHDGLYQGLSTATKDTYDALHMQALPPRGGSGGTSMSDSGSYGQ
SGGEQQSYSTYGNPGSQGYGQASQSYSGYGQTTDSSYGQNYSGYSSYGQSY
SQSYGGYENQKQSSYSQQPYNNQGQQQNMESSGSQGGRAPSYDQPDYGQQD
SYDQQSGYDQHQGSYDEQSNYDQQHDSYSQNQQSYHSQRENYSHHTQDDRR
DVSRYGEDNRGYGGSQGG

In some embodiments, the CAR-IDR is further conjugated to one or more fluorescent tags such as GFP. An exemplary amino acid sequence of an anti-CD19 CAR-IDR including an IDR derived from TAF15 and conjugated thereto a GFP is:

(SEQ ID NO: 10)
MALPVTALLLPLALLLHAARPDIQMTQTTSSLSASLGDRVTISCRASQDIS
KYLNWYQQKPDGTVKLLIYHTSRLHSGVPSRFSGSGSGTDYSLTISNLEQE
DIATYFCQQGNTLPYTFGGGTKLEITGGGGSGGGGSGGGGSEVKLQESGPG
LVAPSQSLSVTCTVSGVSLPDYGVSWIRQPPRKGLEWLGVIWGSETTYYNS
ALKSRLTIIKDNSKSQVFLKMNSLQTDDTAIYYCAKHYYYGGSYAMDYWGQ
GTSVTVSSGSGSEQKLISEEDLGSTTTPAPRPPTPAPTIASQPLSLRPEAC
RPAAGGAVHTRGLDFACDIYIWAPLAGTCGVLLLSLVITLYCSRSKRSRLL
HSDYMNMTPRRPGPTRKHYQPYAPPRDFAAYRSKRGRKKLLYIFKQPFMRP
VQTTQEEDGCSCRFPEEEEGGCELRVKFSRSADAPAYQQGQNQLYNELNLG
RREEYDVLDKRRGRDPEMGGKPQRRKNPQEGLYNELQKDKMAEAYSEIGMK
GERRRGKGHDGLYQGLSTATKDTYDALHMQALPPRGGSGGTSMSDSGSYGQ
SGGEQQSYSTYGNPGSQGYGQASQSYSGYGQTTDSSYGQNYSGYSSYGQSY
SQSYGGYENQKQSSYSQQPYNNQGQQQNMESSGSQGGRAPSYDQPDYGQQD
SYDQQSGYDQHQGSYDEQSNYDQQHDSYSQNQQSYHSQRENYSHHTQDDRR
DVSRYGEDNRGYGGSQGGGSGSMSKGEELFTGVVPILVELDGDVNGHKFSV
RGEGEGDATNGKLTLKFICTTGKLPVPWPTLVTTLTYGVQCFSRYPDHMKR
HDFFKSAMPEGYVQERTISFKDDGTYKTRAEVKFEGDTLVNRIELKGIDFK
EDGNILGHKLEYNFNSHNVYITADKQKNGIKAN
FKIRHNVEDGSVQLADHYQQNTPIGDGPVLLPDNHYLSTQSVLSKDPNEKR
DHMVLLEFVTAAGITHGMDELYK

An exemplary amino acid sequence anti-CD19 CAR-IDR including an IDR derived from EWS is:

(SEQ ID NO: 11)
MALPVTALLLPLALLLHAARPDIQMTQTTSSLSASLGDRVTISCRASQDIS
KYLNWYQQKPDGTVKLLIYHTSRLHSGVPSRFSGSGSGTDYSLTISNLEQE
DIATYFCQQGNTLPYTFGGGTKLEITGGGGSGGGGSGGGGSEVKLQESGPG
LVAPSQSLSVTCTVSGVSLPDYGVSWIRQPPRKGLEWLGVIWGSETTYYNS
ALKSRLTIIKDNSKSQVFLKMNSLQTDDTAIYYCAKHYYYGGSYAMDYWGQ
GTSVTVSSGSGSEQKLISEEDLGSTTTPAPRPPTPAPTIASQPLSLRPEAC
RPAAGGAVHTRGLDFACDIYIWAPLAGTCGVLLLSLVITLYCSRSKRSRLL
HSDYMNMTPRRPGPTRKHYQPYAPPRDFAAYRSKRGRKKLLYIFKQPFMRP
VQTTQEEDGCSCRFPEEEEGGCELRVKFSRSADAPAYQQGQNQLYNELNLG
RREEYDVLDKRRGRDPEMGGKPQRRKNPQEGLYNELQKDKMAEAYSEIGMK
GERRRGKGHDGLYQGLSTATKDTYDALHMQALPPRRTGSGSMGYAQTTQAY
GQQSYGTYGQPTDVSYTQAQTTATYGQTAYATSYGQPPTGYTTPTAPQAYS
QPVQGYGTGAYDITTATVTTTQASYAAQSAYGTQPAYPAYGQQPAATAPTR
PQDGNKPTETSQPQSSTGGYNQPSLGYGQSNYSYPQVPGSYPMQPVTAPPS
YPPTSYSSTQPTSYDQSSYSQQNTYGQPSSYGQQSSYGQQSSYGQQPPTSY
PPQTGSYSQAPSQYSQQSSSYG

In some embodiments, the CAR-IDR is further conjugated to one or more fluorescent tags such as GFP. An exemplary amino acid sequence of an anti-CD19 CAR-IDR including an IDR derived from EWS and a GFP conjugated thereto is:

(SEQ ID NO: 12)
MALPVTALLLPLALLLHAARPDIQMTQTTSSLSASLGDRVTISCRASQDIS
KYLNWYQQKPDGTVKLLIYHTSRLHSGVPSRFSGSGSGTDYSLIISNLEQE
DIATYFCQQGNTLPYTFGGGTKLEITGGGGSGGGGSGGGGSEVKLQESGPG
LVAPSQSLSVTCTVSGVSLPDYGVSWIRQPPRKGLEWLGVIWGSETTYYNS
ALKSRLTIIKDNSKSQVFLKMNSLQTDDTAIYYCAKHYYYGGSYAMDYWGQ
GTSVTVSSGSGSEQKLISEEDLGSTTTPAPRPPTPAPTIASQPLSLRPEAC
RPAAGGAVHTRGLDFACDIYIWAPLAGTCGVLLLSLVITLYCSRSKRSRLL
HSDYMNMTPRRPGPTRKHYQPYAPPRDFAAYRSKRGRKKLLYIFKQPFMRP
VQTTQEEDGCSCRFPEEEEGGCELRVKFSRSADAPAYQQGQNQLYNELNLG
RREEYDVLDKRRGRDPEMGGKPQRRKNPQEGLYNELQKDKMAEAYSEIGMK
GERRRGKGHDGLYQGLSTATKDTYDALHMQALPPRRIGSGSMGYAQTTQAY
GQQSYGTYGQPTDVSYTQAQTTATYGQTAYATSYGQPPTGYTTPTAPQAYS
QPVQGYGTGAYDTTTATVTTTQASYAAQSAYGTQPAYPAYGQQPAATAPTR
PQDGNKPTETSQPQSSTGGYNQPSLGYGQSNYSYPQVPGSYPMQPVTAPPS
YPPTSYSSTQPTSYDQSSYSQQNTYGQPSSYGQQSSYGQQSSYGQQPPTSY
PPQTGSYSQAPSQYSQQSSSYGTSGSGSMSKGEELFTGVVPILVELDGDVN
GHKFSVRGEGEGDATNGKLTLKFICTTGKLPVPWPTLVTTLTYGVQCFSRY
PDHMKRHDFFKSAMPEGYVQERTISFKDDGTYKTRAEVKFEGDTLVNRIEL
KGIDFKEDGNILGHKLEYNFNSHNVYITADKQKNGIKANFKIRHNVEDGSV
QLADHYQQNTPIGDGPVLLPDNHYLSTQSVLSKDPNEKRDHMVLLEFVTAA
GITHGMDELYK

An exemplary amino acid sequence anti-CD19 CAR-IDR including an IDR derived from Nup98 is:

(SEQ ID NO: 13)
MALPVTALLLPLALLLHAARPDIQMTQTTSSLSASLGDRVTISCRASQDIS
KYLNWYQQKPDGTVKLLIYHTSRLHSGVPSRFSGSGSGTDYSLTISNLEQE
DIATYFCQQGNTLPYTFGGGTKLEITGGGGSGGGGSGGGGSEVKLQESGPG
LVAPSQSLSVTCTVSGVSLPDYGVSWIRQPPRKGLEWLGVIWGSETTYYNS
ALKSRLTIIKDNSKSQVFLKMNSLQTDDTAIYYCAKHYYYGGSYAMDYWGQ
GTSVTVSSGSGSEQKLISEEDLGSTTTPAPRPPTPAPTIASQPLSLRPEAC
RPAAGGAVHTRGLDFACDIYIWAPLAGTCGVLLLSLVITLYCSRSKRSRLL
HSDYMNMTPRRPGPTRKHYQPYAPPRDFAAYRSKRGRKKLLYIFKQPFMRP
VQTTQEEDGCSCRFPEEEEGGCELRVKFSRSADAPAYQQGQNQLYNELNLG
RREEYDVLDKRRGRDPEMGGKPQRRKNPQEGLYNELQKDKMAEAYSEIGMK
GERRRGKGHDGLYQGLSTATKDTYDALHMQALPPRGGSGGFNKSFGTPFGG
GTGGFGTTSTFGQNTGFGTTSGGAFGTSAFGSSNNTGGLFGNSQTKPGGLF
GTSSFSQPATSTSTGFGFGISTGTANTLFGTASTGTSLFSSQNNAFAQNKP
TGFGNFGTSTSSGGLFGTTNTTSNPFGSTSGSLFGPSSFTAAPTGTTIKEN
PPTGTDTMVKAGVSTNISTKHQCITAMKEYESKSLEELRLEDYQANRKGPQ
NQVGAGTTTGLFGSSPATSSATGLFSSSTTNSGFAYGQNKTAFGTSTTGFG
TNPGGLFGQQNQQTTSLFSKPFGQATTTQNTGFSFGNTSTIGQPSTNTMGL
FGVTQASQPGGLFGTATNTSTGT

In some embodiments, the CAR-IDR is further conjugated to one or more fluorescent tags such as GFP. An exemplary amino acid sequence of an anti-CD19 CAR-IDR including an IDR derived from Nup98 and a GFP conjugated thereto is:

(SEQ ID NO: 14)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSGSGSEQKLISEEDLGSTTTPAPRPPTPAPTIASQPLS
LRPEACRPAAGGAVHTRGLDFACDIYIWAPLAGTCGVLLLSLVITLYCSR
SKRSRLLHSDYMNMTPRRPGPTRKHYQPYAPPRDFAAYRSKRGRKKLLYI
FKQPFMRPVQTTQEEDGCSCRFPEEEEGGCELRVKFSRSADAPAYQQGQN
QLYNELNLGRREEYDVLDKRRGRDPEMGGKPQRRKNPQEGLYNELQKDKM
AEAYSEIGMKGERRRGKGHDGLYQGLSTATKDTYDALHMQALPPRGGSGG
FNKSFGTPFGGGTGGFGTTSTFGQNTGFGTTSGGAFGTSAFGSSNNTGGL
FGNSQTKPGGLFGTSSFSQPATSTSTGFGFGTSTGTANTLFGTASTGTSL
FSSQNNAFAQNKPTGFGNFGTSTSSGGLFGTTNTTSNPFGSTSGSLFGPS
SFTAAPTGTTIKFNPPTGTDTMVKAGVSTNISTKHQCITAMKEYESKSLE
ELRLEDYQANRKGPQNQVGAGTTTGLFGSSPATSSATGLFSSSTTNSGFA
YGQNKTAFGTSTTGFGTNPGGLFGQQNQQTTSLFSKPFGQATTTQNTGFS
FGNTSTIGQPSTNTMGLFGVTQASQPGGLFGTATNTSTGTGSGSMSKGEE
LFTGVVPILVELDGDVNGHKFSVRGEGEGDATNGKLTLKFICTTGKLPVP
WPTLVTTLTYGVQCFSRYPDHMKRHDFFKSAMPEGYVQERTISFKDDGTY
KTRAEVKFEGDTLVNRIELKGIDFKEDGNILGHKLEYNFNSHNVYITADK
QKNGIKANFKIRHNVEDGSVQLADHYQQNTPIGDGPVLLPDNHYLSTQSV
LSKDPNEKRDHMVLLEFVTAAGITHGMDELYK

An exemplary amino acid sequence anti-CD19 CAR-IDR including an IDR derived from TDP43 is:

(SEQ ID NO: 15)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSGSGSEQKLISEEDLGSTTTPAPRPPTPAPTIASQPLS
LRPEACRPAAGGAVHTRGLDFACDIYIWAPLAGTCGVLLLSLVITLYCSR
SKRSRLLHSDYMNMTPRRPGPTRKHYQPYAPPRDFAAYRSKRGRKKLLYI
FKQPFMRPVQTTQEEDGCSCRFPEEEEGGCELRVKFSRSADAPAYQQGQN
QLYNELNLGRREEYDVLDKRRGRDPEMGGKPQRRKNPQEGLYNELQKDKM
AEAYSEIGMKGERRRGKGHDGLYQGLSTATKDTYDALHMQALPPRGSGSG
SNRQLERSGRFGGNPGGFGNQGGFGNSRGGGAGLGNNQGSNMGGGMNFGA
FSINPAMMAAAQAALQSSWGMMGMLASQQNQSGPSGNNQNQGNMQREPNQ
AFGSGNNSYSGSNSGAAIGWGSASNAGSGSGFNGGFGSSMDSKSSGWGM

In some embodiments, the CAR-IDR is further conjugated to one or more fluorescent tags such as GFP. An exemplary amino acid sequence of an anti-CD19 CAR-IDR including an IDR derived from TDP43 and a GFP conjugated thereto is:

(SEQ ID NO: 16)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSGSGSEQKLISEEDLGSTTTPAPRPPTPAPTIASQPLS
LRPEACRPAAGGAVHTRGLDFACDIYIWAPLAGTCGVLLLSLVITLYCSR
SKRSRLLHSDYMNMTPRRPGPTRKHYQPYAPPRDFAAYRSKRGRKKLLYI
FKQPFMRPVQTTQEEDGCSCRFPEEEEGGCELRVKFSRSADAPAYQQGQN
QLYNELNLGRREEYDVLDKRRGRDPEMGGKPQRRKNPQEGLYNELQKDKM
AEAYSEIGMKGERRRGKGHDGLYQGLSTATKDTYDALHMQALPPRGSGSG
SNRQLERSGRFGGNPGGFGNQGGFGNSRGGGAGLGNNQGSNMGGGMNFGA
FSINPAMMAAAQAALQSSWGMMGMLASQQNQSGPSGNNQNQGNMQREPNQ
AFGSGNNSYSGSNSGAAIGWGSASNAGSGSGFNGGFGSSMDSKSSGWGMT
SGSGSMSKGEELFTGVVPILVELDGDVNGHKFSVRGEGEGDATNGKLTLK
FICTTGKLPVPWPTLVTTLTYGVQCFSRYPDHMKRHDFFKSAMPEGYVQE
RTISFKDDGTYKTRAEVKFEGDTLVNRIELKGIDFKEDGNILGHKLEYNF
NSHNVYITADKQKNGIKANFKIRHNVEDGSVQLADHYQQNTPIGDGPVLL
PDNHYLSTQSVLSKDPNEKRDHMVLLEFVTAAGITHGMDELYK

Another exemplary amino acid sequence anti-CD19 CAR-IDR including a synthetic IDR of SEQ ID NO:6.

(SEQ ID NO: 17)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSGSGSEQKLISEEDLGSTTTPAPRPPTPAPTIASQPLS
LRPEACRPAAGGAVHTRGLDFACDIYIWAPLAGTCGVLLLSLVITLYCSR
SKRSRLLHSDYMNMTPRRPGPTRKHYQPYAPPRDFAAYRSKRGRKKLLYI
FKQPFMRPVQTTQEEDGCSCRFPEEEEGGCELRVKFSRSADAPAYQQGQN
QLYNELNLGRREEYDVLDKRRGRDPEMGGKPQRRKNPQEGLYNELQKDKM
AEAYSEIGMKGERRRGKGHDGLYQGLSTATKDTYDALHMQALPPRGSGSS
KGPGRGDSPYSGRGDSPYSGRGDSPYSGRGDSPYSGRGDSPYSGRGDSPY
SGRGDSPYSGRGDSPYSGRGDSPYSGRGDSPYSGY

An exemplary amino acid sequence of an anti-CD19 CAR-IDR including a synthetic IDR of SEQ ID NO:6 and a GFP conjugated thereto is:

(SEQ ID NO: 18)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSGSGSEQKLISEEDLGSTTTPAPRPPTPAPTIASQPLS
LRPEACRPAAGGAVHTRGLDFACDIYIWAPLAGTCGVLLLSLVITLYCSR
SKRSRLLHSDYMNMTPRRPGPTRKHYQPYAPPRDFAAYRSKRGRKKLLYI
FKQPFMRPVQTTQEEDGCSCRFPEEEEGGCELRVKFSRSADAPAYQQGQN
QLYNELNLGRREEYDVLDKRRGRDPEMGGKPQRRKNPQEGLYNELQKDKM
AEAYSEIGMKGERRRGKGHDGLYQGLSTATKDTYDALHMQALPPRGSGSS
KGPGRGDSPYSGRGDSPYSGRGDSPYSGRGDSPYSGRGDSPYSGRGDSPY
SGRGDSPYSGRGDSPYSGRGDSPYSGRGDSPYSGYTSGSGSMSKGEELFT
GVVPILVELDGDVNGHKFSVRGEGEGDATNGKLTLKFICTTGKLPVPWPT
LVTTLTYGVQCFSRYPDHMKRHDFFKSAMPEGYVQERTISFKDDGTYKTR
AEVKFEGDTLVNRIELKGIDFKEDGNILGHKLEYNFNSHNVYITADKQKN
GIKANFKIRHNVEDGSVQLADHYQQNTPIGDGPVLLPDNHYLSTQSVLSK
DPNEKRDHMVLLEFVTAAGITHGMDELYK

In some forms, the function of the fusion peptide including one or more IDR domains is compared to a control peptide, e.g., one having the same CAR domain but without an IDR domain. In some cases, a fluorescent tag is added to track or assess the control peptide, for example, a GFP can be fused to the C-terminus of the control peptide. An exemplary control CAR peptide with a C-terminal GFP, is an anti-CD19 CAR-GFP having the following amino acid sequence.

(SEQ ID NO: 19)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSGSGSEQKLISEEDLGSTTTPAPRPPTPAPTIASQPLS
LRPEACRPAAGGAVHTRGLDFACDIYIWAPLAGTCGVLLLSLVITLYCSR
SKRSRLLHSDYMNMTPRRPGPTRKHYQPYAPPRDFAAYRSKRGRKKLLYI
FKQPFMRPVQTTQEEDGCSCRFPEEEEGGCELRVKFSRSADAPAYQQGQN
QLYNELNLGRREEYDVLDKRRGRDPEMGGKPQRRKNPQEGLYNELQKDKM
AEAYSEIGMKGERRRGKGHDGLYQGLSTATKDTYDALHMQALPPRISGSG
SMSKGEELFTGVVPILVELDGDVNGHKFSVRGEGEGDATNGKLTLKFICT
TGKLPVPWPTLVTTLTYGVQCFSRYPDHMKRHDFFKSAMPEGYVQERTIS
FKDDGTYKTRAEVKFEGDTLVNRIELKGIDFKEDGNILGHKLEYNFNSHN
VYITADKQKNGIKANFKIRHNVEDGSVQLADHYQQNTPIGDGPVLLPDNH
YLSTQSVLSKDPNEKRDHMVLLEFVTAAGITHGMDELYK

In some forms, the fusion peptide is a CAR including one or more IDR domains. An exemplary CAR-IDR is an anti-CD22 CAR-IDR. An exemplary amino acid sequence anti-CD22 CAR-IDR including an IDR derived from FUS is:

(SEQ ID NO: 20)
MALPVTALLLPLALLLHAARPDIQMTQTTSSLSASLGDRVTISCRASQDI
SNYLNWYQQKPDGTVKLLIYYTSILHSGVPSRFSGSGSGTDYSLTISNLE
QEDFATYFCQQGNTLPWTFGGGTKLEIKGGGGSGGGGSGGGGSEVQLVES
GGGLVKPGGSLKLSCAASGFAFSIYDMSWVRQTPEKRLEWVAYISSGGGT
TYYPDTVKGRFTISRDNAKNTLYLQMSSLKSEDTAMYYCARHSGYGSSYG
VLFAYWGQGTLVTVSAGSGSEQKLISEEDLGSTTTPAPRPPTPAPTIASQ
PLSLRPEACRPAAGGAVHIRGLDFACDIYIWAPLAGTCGVLLLSLVITLY
CSRSKRSRLLHSDYMNMTPRRPGPTRKHYQPYAPPRDFAAYRSRVKFSRS
ADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHM
QALPPRTSGSGSGSGSMASNDYTQQATQSYGAYPTQPGQGYSQQSSQPYG
QQSYSGYSQSTDTSGYGQSSYSSYGQSQNTGYGTQSTPQGYGSTGGYGSS
QSSQSSYGQQSSYPGYGQQPAPSSTSGSYGSSSQSSSYGQPQSGSYSQQP
SYGGQQQSYGQQQSYNPPQGYGQQNQYNSSSGGGGGGGGGGNYGQDQSSM
SSGGGSGGGYGNQDQSGGGGSGGYGQQDRG

Another exemplary amino acid sequence anti-CD22 CAR-IDR including an IDR derived from EWS is:

(SEQ ID NO: 21)
MALPVTALLLPLALLLHAARPDIQMTQTTSSLSASLGDRVTISCRASQDI
SNYLNWYQQKPDGTVKLLIYYTSILHSGVPSRFSGSGSGTDYSLTISNLE
QEDFATYFCQQGNTLPWTFGGGTKLEIKGGGGSGGGGSGGGGSEVQLVES
GGGLVKPGGSLKLSCAASGFAFSIYDMSWVRQTPEKRLEWVAYISSGGGT
TYYPDTVKGRFTISRDNAKNTLYLQMSSLKSEDTAMYYCARHSGYGSSYG
VLFAYWGQGTLVTVSAGSGSEQKLISEEDLGSTTTPAPRPPTPAPTIASQ
PLSLRPEACRPAAGGAVHTRGLDFACDIYIWAPLAGTCGVLLLSLVITLY
CSRSKRSRLLHSDYMNMTPRRPGPTRKHYQPYAPPRDFAAYRSRVKFSRS
ADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHM
QALPPRRTGSGSMGYAQTTQAYGQQSYGTYGQPTDVSYTQAQTTATYGQT
AYATSYGQPPTGYTTPTAPQAYSQPVQGYGTGAYDTTTATVTTTQASYAA
QSAYGTQPAYPAYGQQPAATAPTRPQDGNKPTETSQPQSSTGGYNQPSLG
YGQSNYSYPQVPGSYPMQPVTAPPSYPPTSYSSTQPTSYDQSSYSQQNTY
GQPSSYGQQSSYGQQSSYGQQPPTSYPPQTGSYSQAPSQYSQQSSSYG

Another exemplary amino acid sequence anti-CD22 CAR-IDR including an IDR derived from TAF15 is:

(SEQ ID NO: 22)
MALPVTALLLPLALLLHAARPDIQMTQTTSSLSASLGDRVTISCRASQDI
SNYLNWYQQKPDGTVKLLIYYTSILHSGVPSRFSGSGSGTDYSLTISNLE
QEDFATYFCQQGNTLPWTFGGGTKLEIKGGGGSGGGGSGGGGSEVQLVES
GGGLVKPGGSLKLSCAASGFAFSIYDMSWVRQTPEKRLEWVAYISSGGGT
TYYPDTVKGRFTISRDNAKNTLYLQMSSLKSEDTAMYYCARHSGYGSSYG
VLFAYWGQGTLVTVSAGSGSEQKLISEEDLGSTTTPAPRPPTPAPTIASQ
PLSLRPEACRPAAGGAVHTRGLDFACDIYIWAPLAGTCGVLLLSLVITLY
CSRSKRSRLLHSDYMNMTPRRPGPTRKHYQPYAPPRDFAAYRSRVKFSRS
ADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHM
QALPPRISGSGSMMSDSGSYGQSGGEQQSYSTYGNPGSQGYGQASQSYSG
YGQTTDSSYGQNYSGYSSYGQSYSQSYGGYENQKQSSYSQQPYNNQGQQQ
NMESSGSQGGRAPSYDQPDYGQQDSYDQQSGYDQHQGSYDEQSNYDQQHD
SYSQNQQSYHSQRENYSHHTQDDRRDVSRYGEDNRGYGGSQGG

In some forms, the function of the fusion peptide including one or more IDR domains is compared to a control peptide, e.g., one having the same CAR domain but without an IDR domain. In some cases, a fluorescent tag is added to track or assess the control peptide, for example, a GFP can be fused to the C-terminus of the control peptide. An exemplary control CAR peptide with a C-terminal GFP, is an anti-CD22 CAR-GFP having the following amino acid sequence.

(SEQ ID NO: 23)
MALPVTALLLPLALLLHAARPDIQMTQTTSSLSASLGDRVTISCRASQDI
SNYLNWYQQKPDGTVKLLIYYTSILHSGVPSRFSGSGSGTDYSLTISNLE
QEDFATYFCQQGNTLPWTFGGGTKLEIKGGGGSGGGGSGGGGSEVQLVES
GGGLVKPGGSLKLSCAASGFAFSIYDMSWVRQTPEKRLEWVAYISSGGGT
TYYPDTVKGRFTISRDNAKNTLYLQMSSLKSEDTAMYYCARHSGYGSSYG
VLFAYWGQGTLVTVSAGSGSEQKLISEEDLGSTTTPAPRPPTPAPTIASQ
PLSLRPEACRPAAGGAVHTRGLDFACDIYIWAPLAGTCGVLLLSLVITLY
CSRSKRSRLLHSDYMNMTPRRPGPTRKHYQPYAPPRDFAAYRSRVKFSRS
ADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHM
QALPPRTSGSGSMSKGEELFTGVVPILVELDGDVNGHKFSVRGEGEGDAT
NGKLTLKFICTTGKLPVPWPTLVTTLTYGVQCFSRYPDHMKRHDFFKSAM
PEGYVQERTISFKDDGTYKTRAEVKFEGDILVNRIELKGIDFKEDGNILG
HKLEYNFNSHNVYITADKQKNGIKANFKIRHNVEDGSVQLADHYQQNTPI
GDGPVLLPDNHYLSTQSVLSKDPNEKRDHMVLLEFVTAAGITHGMDELYK

In some forms, the fusion peptide is a CAR including one or more IDR domains. An exemplary CAR-IDR is an anti-HER2 CAR-IDR. An exemplary amino acid sequence anti-HER2 CAR-IDR including an IDR derived from FUS is:

(SEQ ID NO: 24)
METDTLLLWVLLLWVPGSTGDKYLLPTAAAGLLLLAAQPAMAQVQLVQSG
AEVKKPGESLKISCKGSGYSFTSYWIAWVRQMPGKGLEYMGLIYPGDSDT
KYSPSFQGQVTISVDKSVSTAYLQWSSLKPSDSAVYFCARHDVGYCTDRT
CAKWPEYFQHWGQGTLVTVSSGGGGDGGGGSGGGGSQSVLTQPPSVSAAP
GQKVTISCSGSSSNIGNNYVSWYQQLPGTAPKLLIYDHTNRPAGVPDRFS
GSKSGTSASLAISGFRSEDEADYYCASWDYTLSGWVFGGGTKLTVLGGSG
SEQKLISEEDLGSTTTPAPRPPTPAPTIASQPLSLRPEACRPAAGGAVHT
RGLDFACDFWVLVVVGGVLACYSLLVTVAFIIFWVSRSKRSRLLHSDYMN
MTPRRPGPTRKHYQPYAPPRDFAAYRSKRGRKKLLYIFKQPFMRPVQTTQ
EEDGCSCRFPEEEEGGCELRVKFSRSADAPAYQQGQNQLYNELNLGRREE
YDVLDKRRGRDPEMGGKPQRRKNPQEGLYNELQKDKMAEAYSEIGMKGER
RRGKGHDGLYQGLSTATKDTYDALHMQALPPRTSGSGSGSGSMASNDYTQ
QATQSYGAYPTQPGQGYSQQSSQPYGQQSYSGYSQSTDTSGYGQSSYSSY
GQSQNTGYGTQSTPQGYGSTGGYGSSQSSQSSYGQQSSYPGYGQQPAPSS
TSGSYGSSSQSSSYGQPQSGSYSQQPSYGGQQQSYGQQQSYNPPQGYGQQ
NQYNSSSGGGGGGGGGGNYGQDQSSMSSGGGSGGGYGNQDQSGGGGSGGY
GQQDRG

An exemplary amino acid sequence anti-HER2 CAR-IDR including an IDR derived from TAF15 is:

(SEQ ID NO: 25)
METDTLLLWVLLLWVPGSTGDKYLLPTAAAGLLLLAAQPAMAQVQLVQSG
AEVKKPGESLKISCKGSGYSFTSYWIAWVRQMPGKGLEYMGLIYPGDSDT
KYSPSFQGQVTISVDKSVSTAYLQWSSLKPSDSAVYFCARHDVGYCTDRT
CAKWPEYFQHWGQGTLVTVSSGGGGSGGGGSGGGGSQSVLTQPPSVSAAP
GQKVTISCSGSSSNIGNNYVSWYQQLPGTAPKLLIYDHTNRPAGVPDRFS
GSKSGTSASLAISGFRSEDEADYYCASWDYTLSGWVFGGGTKLTVLGGSG
SEQKLISEEDLGSTTTPAPRPPTPAPTIASQPLSLRPEACRPAAGGAVHT
RGLDFACDFWVLVVVGGVLACYSLLVTVAFIIFWVSRSKRSRLLHSDYMN
MTPRRPGPTRKHYQPYAPPRDFAAYRSKRGRKKLLYIFKQPFMRPVQTTQ
EEDGCSCRFPEEEEGGCELRVKFSRSADAPAYQQGQNQLYNELNLGRREE
YDVLDKRRGRDPEMGGKPQRRKNPQEGLYNELQKDKMAEAYSEIGMKGER
RRGKGHDGLYQGLSTATKDTYDALHMQALPPRTSGSGSMMSDSGSYGQSG
GEQQSYSTYGNPGSQGYGQASQSYSGYGQTTDSSYGQNYSGYSSYGQSYS
QSYGGYENQKQSSYSQQPYNNQGQQQNMESSGSQGGRAPSYDQPDYGQQD
SYDQQSGYDQHQGSYDEQSNYDQQHDSYSQNQQSYHSQRENYSHHTQDDR
RDVSRYGEDNRGYGGSQGG

An exemplary amino acid sequence anti-HER2 CAR-IDR including an IDR derived from EWS is:

(SEQ ID NO: 26)
METDTLLLWVLLLWVPGSTGDKYLLPTAAAGLLLLAAQPAMAQVQLVQSG
AEVKKPGESLKISCKGSGYSFTSYWIAWVRQMPGKGLEYMGLIYPGDSDT
KYSPSFQGQVTISVDKSVSTAYLQWSSLKPSDSAVYFCARHDVGYCTDRT
CAKWPEYFQHWGQGTLVTVSSGGGGSGGGGSGGGGSQSVLTQPPSVSAAP
GQKVTISCSGSSSNIGNNYVSWYQQLPGTAPKLLIYDHTNRPAGVPDRFS
GSKSGTSASLAISGFRSEDEADYYCASWDYTLSGWVFGGGTKLTVLGGSG
SEQKLISEEDLGSTTTPAPRPPTPAPTIASQPLSLRPEACRPAAGGAVHT
RGLDFACDFWVLVVVGGVLACYSLLVTVAFIIFWVSRSKRSRLLHSDYMN
MTPRRPGPTRKHYQPYAPPRDFAAYRSKRGRKKLLYIFKQPFMRPVQTTQ
EEDGCSCRFPEEEEGGCELRVKFSRSADAPAYQQGQNQLYNELNLGRREE
YDVLDKRRGRDPEMGGKPQRRKNPQEGLYNELQKDKMAEAYSEIGMKGER
RRGKGHDGLYQGLSTATKDTYDALHMQALPPRRIGSGSMGYAQTTQAYGQ
QSYGTYGQPTDVSYTQAQTTATYGQTAYATSYGQPPTGYTTPTAPQAYSQ
PVQGYGTGAYDTTTATVTTTQASYAAQSAYGTQPAYPAYGQQPAATAPTR
PQDGNKPTETSQPQSSTGGYNQPSLGYGQSNYSYPQVPGSYPMQPVTAPP
SYPPTSYSSTQPTSYDQSSYSQQNTYGQPSSYGQQSSYGQQSSYGQQPPT
SYPPQTGSYSQAPSQYSQQSSSYG

In some forms, the function of the fusion peptide including one or more IDR domains is compared to a control peptide, e.g., one having the same CAR domain but without an IDR domain. In some cases, a fluorescent tag is added to track or assess the control peptide, for example, a GFP can be fused to the C-terminus of the control peptide. An exemplary control CAR peptide with a C-terminal GFP, is an anti-HER2 CAR-GFP having the following amino acid sequence.

(SEQ ID NO: 27)
METDTLLLWVLLLWVPGSTGDKYLLPTAAAGLLLLAAQPAMAQVQLVQSG
AEVKKPGESLKISCKGSGYSFTSYWIAWVRQMPGKGLEYMGLIYPGDSDT
KYSPSFQGQVTISVDKSVSTAYLQWSSLKPSDSAVYFCARHDVGYCTDRT
CAKWPEYFQHWGQGTLVTVSSGGGGSGGGGSGGGGSQSVLTQPPSVSAAP
GQKVTISCSGSSSNIGNNYVSWYQQLPGTAPKLLIYDHTNRPAGVPDRFS
GSKSGTSASLAISGFRSEDEADYYCASWDYTLSGWVFGGGTKLTVLGGSG
SEQKLISEEDLGSTTTPAPRPPTPAPTIASQPLSLRPEACRPAAGGAVHT
RGLDFACDFWVLVVVGGVLACYSLLVTVAFIIFWVSRSKRSRLLHSDYMN
MTPRRPGPTRKHYQPYAPPRDFAAYRSKRGRKKLLYIFKQPFMRPVQTTQ
EEDGCSCRFPEEEEGGCELRVKFSRSADAPAYQQGQNQLYNELNLGRREE
YDVLDKRRGRDPEMGGKPQRRKNPQEGLYNELQKDKMAEAYSEIGMKGER
RRGKGHDGLYQGLSTATKDTYDALHMQALPPRTSGSGSMSKGEELFTGVV
PILVELDGDVNGHKFSVRGEGEGDATNGKLTLKFICTTGKLPVPWPTLVT
TLTYGVQCFSRYPDHMKRHDFFKSAMPEGYVQERTISFKDDGTYKTRAEV
KFEGDTLVNRIELKGIDFKEDGNILGHKLEYNFNSHNVYITADKQKNGIK
ANFKIRHNVEDGSVQLADHYQQNTPIGDGPVLLPDNHYLSTQSVLSKDPN
EKRDHMVLLEFVTAAGITHGMDELYK.

### C. Nucleic Acids

Nucleic acids and vectors encoding or expressing IDR proteins and IDR fusion proteins are also described.

### 1. Isolated Nucleic Acid Molecules of IDR or IDR-Fusion Peptides

Isolated nucleic acid sequences encoding IDR polypeptides and IDR fusion peptides are disclosed. In some embodiments, the isolated nucleic acid sequences encode a CAR-IDR, including a CAR fused with an IDR domain. In preferred embodiments, an isolated nucleic acid sequence encodes a CAR fused with an IDR domain including any one of SEQ ID NOs: 1-27.

The term “isolated nucleic acid” refers to a nucleic acid that is separated from other nucleic acid molecules that are present in a mammalian genome, including nucleic acids that normally flank one or both sides of the nucleic acid in a mammalian genome. An isolated nucleic acid can be, for example, a DNA molecule, provided one of the nucleic acid sequences normally found immediately flanking that DNA molecule in a naturally occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence encoding an IDR polypeptide or IDR fusion peptide. Thus, nucleic acids encoding SEQ ID NOs:1-27, and fragments and variants thereof, in sense and antisense, and in single stranded and double stranded forms, are provided. The nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

### 2. Vectors Expressing or Encoding IDR or IDR-Fusion Peptides

In some embodiments, nucleic acids encoding IDR or IDR-Fusion Peptides are present within vectors. In some embodiments, the vectors encode or express a CAR-IDR, including a CAR fused with an IDR domain. In preferred embodiments, a vector encodes or expresses a CAR fused with an IDR domain including any one of SEQ ID NOs: 1-27.

Vectors including an isolated polynucleotide encoding an IDR polypeptide and/or fusion polypeptide including an IDR domain and one or more heterologous domains for the expression of an IDR or IDR fusion peptide within a host cell are described.

The term “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). A vector can be a DNA vector or an RNA vector. In some embodiments, a vector is a DNA plasmid. One of ordinary skill in the art can construct a vector of the application through standard recombinant techniques in view of the present disclosure.

In some embodiments, the vector including nucleic acids encoding an IDR domain or IDR fusion protein is an expression vector. The term “expression vector” refers to any type of genetic construct including a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

In some embodiments, vectors contain one or more regulatory sequences. The term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

In some embodiments, the vector is a non-viral vector. Examples of non-viral vectors include, but are not limited to, DNA plasmids, bacterial artificial chromosomes, yeast artificial chromosomes, bacteriophages, etc. Examples of non-viral vectors include, but are not limited to, RNA replicon, mRNA replicon, modified mRNA replicon or self-amplifying mRNA, closed linear deoxyribonucleic acid, e.g., a linear covalently closed DNA, e.g., a linear covalently closed double stranded DNA molecule. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically include an origin of replication, a multiple cloning site, and a selectable marker, which for example, can be an antibiotic resistance gene. Examples of suitable DNA plasmids that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC®. complete baculovirus expression system (Thermo Fisher Scientific), which can be used for production and/or expression in insect cells; pcDNA™. or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNA™, pcDNA3™, pVAX, pVAX-1, ADVAX, NTC8454, etc. In some embodiments, an expression vector is based on pVAX-1, which can be further modified to optimize protein expression in mammalian cells. pVAX-1 is a commonly used plasmid in DNA vaccines, and contains a strong human immediate early cytomegalovirus (CMV-IE) promoter followed by the bovine growth hormone (bGH)-derived polyadenylation sequence (pA). pVAX-1 further contains a pUC origin of replication and a kanamycin resistance gene driven by a small prokaryotic promoter that allows for bacterial plasmid propagation.

In some embodiments, the vector is a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, arenavirus viral vectors, replication-deficient arenavirus viral vectors or replication-competent arenavirus viral vectors, bi-segmented or tri-segmented arenavirus, infectious arenavirus viral vectors, nucleic acids which include an arenavirus genomic segment wherein one open reading frame of the genomic segment is deleted or functionally inactivated (and replaced by a nucleic acid encoding a PC1-CTT polypeptide or another therapeutic polypeptide as described herein), arenavirus such as lymphocytic choriomeningitidis virus (LCMV), e.g., clone 13 strain or MP strain, and arenavirus such as Junin virus e.g., Candid #1 strain, etc.

In some embodiments, the viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other embodiments, an adenovirus vector is a rhAd vector, e.g., rhAd51, rhAd52 or rhAd53. In some embodiments, a recombinant viral vector is prepared using methods known in the art in view of the present disclosure. For example, in view of the degeneracy of the genetic code, several nucleic acid sequences can be designed that encode the same polypeptide. In some embodiments, a polynucleotide encoding an IDR polypeptide or IDR-fusion polypeptide is codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

In some embodiments, the vectors, e.g., a DNA plasmid or a viral vector (particularly an adenoviral vector), include any regulatory elements to establish conventional function(s) of the vector, including but not limited to replication and expression of the IDR polypeptide or IDR-fusion polypeptide encoded by the polynucleotide sequence of the vector.

### 3. Regulatory Elements

In some embodiments, the disclosed nucleic acids, including RNAs and DNAs such as DNA vectors expressing or encoding an IDR polypeptide or IDR-fusion polypeptide include one or more regulatory elements.

Regulatory elements include, but are not limited to, a promoter, an enhancer, a polyadenylation signal, translation stop codon, a ribosome binding element, a transcription terminator, selection markers, origin of replication, etc. An isolated nucleic acid can be, and a vector can include one or more expression cassettes. An “expression cassette” is part of a nucleic acid such as a vector that directs the cellular machinery to make RNA and protein. An expression cassette typically includes three components: a promoter sequence, an open reading frame, and a 3-untranslated region (UTR) optionally including a polyadenylation signal. An open reading frame (ORF) is a reading frame that contains a coding sequence of a protein of interest (e.g., IDR polypeptide or IDR-fusion polypeptide, etc.) from a start codon to a stop codon. Regulatory elements of the expression cassette can be operably linked to a polynucleotide sequence encoding an IDR polypeptide or IDR-fusion polypeptide.

As used herein, the term “operably linked” is to be taken in its broadest reasonable context, and refers to a linkage of polynucleotide (or polypeptide, etc.) elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

a. Promotors

The disclosed nucleic acids, including vectors, can include a promoter sequence, preferably within an expression cassette, to control expression of an IDR polypeptide or IDR-fusion polypeptide. The term “promoter” is used in its conventional sense and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding an IDR polypeptide or IDR-fusion polypeptide within an expression cassette.

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metallothionein.

A promoter can also be a tissue specific promoter, such as a kidney specific promoter, preferably a kidney epithelial cell promoter, which can be natural or synthetic. Examples include, but are not limited to, the CDH 16 promoter, which is mostly kidney specific (it is also expressed in the thyroid) (Igarashi, et al., Am J Physiol., 277(4):F599-610 (1999)); the Pax-8 promoter, which is also expressed primarily in the kidney as well as in the thyroid (Dehbi, et al., EMBO J., 15(16):4297-306 (1996)); the aquaporin 2 promoter, which drives expression specifically in principal cells of the renal collecting duct (which are the target of Tolvaptan) (Stricklett, et al., Exp Nephrol., 7(1):67-74 (1999)), and kidney tubule-specific promoters in association with gene delivery viral vectors (Watanabe, et al., PloS one, vol. 12,3 e0168638 (2017)).

In some embodiments, the promoter is a strong eukaryotic promoter, such as cytomegalovirus immediate early (CMV-IE) promoter.

b. Other Expression Control Elements

In some embodiments, the nucleic acids, including vectors, include additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for an IDR polypeptide or IDR-fusion polypeptide within an expression cassette of the vector. Enhancer sequences are regulatory DNA sequences that, when bound by transcription factors, enhance the transcription of an associated gene. An enhancer sequence is preferably located upstream of the polynucleotide sequence encoding an IDR polypeptide or IDR-fusion polypeptide, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human beta-globin polyadenylation signal. Preferably, a polyadenylation signal is a bovine growth hormone (bGH) polyadenylation signal or a SV40 polyadenylation signal.

Any enhancer sequence known to those skilled in the art in view of the present disclosure can be used. For example, an enhancer sequence can be a human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein AI precursor (ApoAI), untranslated R-U5 domain of the human T-cell leukemia virus type 1 (HTLV-1) long terminal repeat (LTR), a splicing enhancer, a synthetic rabbit beta-globin intron, or any combination thereof. Preferably, an enhancer sequence is a composite sequence of three consecutive elements of the untranslated R-U5 domain of HTLV-1 LTR, rabbit beta-globin intron, and a splicing enhancer, which is referred to herein as “a triple enhancer sequence.”

A vector can include a polynucleotide sequence encoding a signal peptide sequence. Preferably, the polynucleotide sequence encoding the signal peptide sequence is located upstream of the polynucleotide sequence encoding an IDR polypeptide or IDR-fusion polypeptide. Signal peptides typically direct localization of a protein, facilitate secretion of the protein from the cell in which it is produced, and/or improve expression the therapeutic polypeptide when expressed from the vector, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as the Ig heavy chain gamma signal peptide SPIgG or the Ig heavy chain epsilon signal peptide SPIgE.

A vector, such as a DNA plasmid, can also include a bacterial origin of replication and an antibiotic resistance expression cassette for selection and maintenance of the plasmid in bacterial cells, e.g., E. coli. Bacterial origins of replication and antibiotic resistance cassettes can be located in a vector in the same orientation as the expression cassette encoding an IDR polypeptide or IDR-fusion polypeptide, or in the opposite (reverse) orientation. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC.

Expression cassettes for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest, e.g., an IDR polypeptide or IDR-fusion polypeptide. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kan^{r}), ampicillin resistance gene (Amp^{r}), and tetracycline resistance gene (Tet^{r}), as well as genes conferring resistance to chloramphenicol, bleomycin, spectinomycin, carbenicillin, etc.

An expression vector can include a tag sequence, such as those discussed above.

### D. Host Cells

In some embodiments, polypeptides, nucleic acids, or vectors encoding IDR polypeptide or IDR-fusion polypeptides are present within a host cells. In some embodiments, the cells include nucleic acids or vectors or genes that encode or express a CAR-IDR, including a CAR fused with an IDR domain. In preferred embodiments, the cells include nucleic acids or vectors or genes that encode or express a CAR fused with an IDR domain having SEQ ID NOs. 7-27. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell) can be used to, for example, produce the IDR polypeptide or IDR-fusion polypeptides described herein.

In some forms, the cell is from an established cell line, or a primary cell. The term “primary cell,” refers to cells and cell cultures derived from a subject and allowed to grow in vitro for a limited number of passages, i.e., splitting, of the culture.

### 1. Human Cells

In some embodiments, cells are obtained from a human subject.

Therefore, human cells expressing and/or including IDR-fusion polypeptides are described. In preferred embodiments, the human cells include or express a CAR-IDR, including a CAR fused with an IDR domain. In preferred embodiments, the human cells include or express a CAR fused with an IDR domain including one or more of SEQ ID NOs.:1-27. For example, in some forms, the cells are autologous cells, i.e., cells obtained from a subject prior to introduction of the IDR polypeptides or IDR-fusion polypeptides, and/or nucleic acids, or vectors encoding IDR polypeptides or IDR-fusion polypeptides, and re-introduction to the same subject following modification. In other forms, the cells are heterologous cells, i.e., cells obtained from a different subject than the intended recipient. In some forms, the cells are frozen prior to or after introduction of the IDR polypeptides or IDR-fusion polypeptides, and/or nucleic acids, or vectors encoding IDR polypeptides or IDR-fusion polypeptides. Methods and compositions for freezing and thawing viable eukaryotic cells are known in the art. In some forms, the cells are autologous immune cells, such as T cells or progenitor cells/stem cells.

In some forms, cells are obtained from a healthy subject. In other forms, cells are obtained from a subject identified as having or at risk of having a disease or disorder, such as cancer and/or an auto-immune disease.

In preferred embodiments, the introduction of the IDR polypeptides or IDR-fusion polypeptides to the cells occurs through genetic modification of the cells. In some embodiments, genetic modification of the cell includes introduction of nucleic acids, or vectors encoding IDR polypeptides or IDR-fusion polypeptides to the cell for expression of the IDR polypeptides or IDR-fusion polypeptides within the cell. In some embodiments, genetic modification of the cell includes transduction with a transposon encoding an IDR polypeptide or IDR-fusion polypeptide. In an exemplary embodiment, a CAR-IDR fusion peptide is introduced into a cell in vitro by transduction of the cell with a nucleic acid encoding a transposon including the CAR-IDR. Therefore, genetically modified (transgenic) cells including IDR proteins, or IDR-fusion proteins according to the described compositions are described.

a. T Cells

In some forms, the cells are human immune cells, such as T cells. Therefore, human T cells that include or express IDR-fusion polypeptides are described. In some forms, prior to expansion and genetic modification, T cells are obtained from a diseased or healthy subject. T cells can be obtained from a number of samples, including peripheral blood mononuclear cells, bone marrow, lymph node tissue, cord blood, thymus tissue, tissue from a site of infection, ascites, pleural effusion, spleen tissue, and tumors. In some forms, T cells are obtained from a unit of blood collected from a subject using any number of techniques known to the skilled artisan, such as FICOLL™ separation. In one preferred form, cells from the circulating blood of an individual are obtained by apheresis. The apheresis product typically contains lymphocytes, including T cells, monocytes, granulocytes, B cells, other nucleated white blood cells, red blood cells, and platelets. The cells collected by apheresis can be washed to remove the plasma fraction and to place the cells in an appropriate buffer or media for subsequent processing steps. In some forms, the cells are washed with phosphate buffered saline (PBS). In some forms, the wash solution lacks calcium and can lack magnesium or can lack many if not all divalent cations. After washing, the cells can be resuspended in a variety of biocompatible buffers, such as, for example, Ca²⁺-free, Mg²⁺-free PBS, PLASMALYTE A, or other saline solution with or without buffer. Alternatively, the undesirable components of the apheresis sample are removed, and the cells directly resuspended in culture media.

In some forms, T cells are isolated from peripheral blood lymphocytes by lysing the red blood cells and depleting the monocytes, for example, by centrifugation through a PERCOLL™ gradient or by counterflow centrifugal elutriation. In specific forms, a specific subpopulation of T cells, such as CD3+, CD28+, CD4+, CD8+, CD45RA+, and CD45RO+ T cells, is further isolated by positive or negative selection techniques. For example, in some forms, T cells are isolated by incubation with anti-CD3/anti-CD28-conjugated beads, such as DYNABEADS® M-450 CD3/CD28 T, for a time period sufficient for positive selection of the desired T cells.

In some embodiments, IDR-CAR cells have one or more of increased secretion of one or more cytotoxic factors optionally included IFNγ, Perforin, FasL, Granzyme A, and/or Granzyme B; activation of LAT and/or pathway(s) downstream thereof, optionally comprising SLP76 activation, ERK activation, RAS activation, and/or actin polymerization, optionally wherein LAT activation comprises LAT phosphorylation; and/or reduction of one or more T cell exhaustion markers optionally TIM3 and/or LAG3, relative to, for example, a control cell. Such a control cell can be a cell of the same cell type and the same heterologous fusion protein, but lacking the IDR sequence(s).

b. Delivery Vehicles

Any of the disclosed compositions including, but not limited to IDR polypeptides or IDR-fusion proteins, such as CAR-IDR and/or nucleic acids, can be delivered to target cells using a delivery vehicle. The delivery vehicles can be, for example, polymeric particles, inorganic particles, silica particles, liposomes, micelles, multilamellar vesicles, etc.

Delivery vehicles may be microparticles or nanoparticles. Nanoparticles are often utilized for intertissue application, penetration of cells, and certain routes of administration. The nanoparticles may have any desired size for the intended use. The nanoparticles may have any diameter from 10 nm up to about 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In some embodiments the nanoparticles can have a diameter less than 400 nm, less than 300 nm, or less than 200 nm. The range can be between 50 nm and 300 nm.

Thus, in some embodiments, the delivery vehicles are nanoscale compositions, for example, 10 nm up to, but not including, about 1 micron. However, it will be appreciated that in some embodiments, and for some uses, the particles can be smaller, or larger (e.g., microparticles, etc.). Although many of the compositions disclosed herein are referred to as nanoparticle or nanocarrier compositions, it will be appreciated that in some embodiments and for some uses the carrier can be somewhat larger than nanoparticles. Such compositions can be referred to as microparticulate compositions. For example, a nanocarriers according to the present disclosure may be a microparticle. Microparticles can a diameter between, for example, 0.1 and 100 μm in size.

### E. Pharmaceutical Compositions

Pharmaceutical compositions containing nucleic acids encoding, or a genetically modified cell or a population of genetically modified cells expressing, IDR polypeptides or IDR-fusion proteins such as CAR-IDR are provided.

In some embodiments, the pharmaceutical compositions include one or more of a pharmaceutically acceptable buffer, carrier, diluent, or excipients. In some forms, the pharmaceutical compositions include a specific number or population of cells, for example, expanded by culturing and expanding an isolated genetically modified cell (e.g., CAR-IDR T cell), e.g., a homogenous population. Therefore, in some embodiments, pharmaceutical compositions include a homogenous population of modified cells including and/or expressing an IDR peptide or IDR-fusion peptide, such as a CAR-IDR. In other forms, the pharmaceutical compositions include populations of cells that contain variable or different genetically modified cells, e.g., a heterogeneous population. In some forms, the pharmaceutical compositions include cells that are bispecific or multi-specific. In some embodiments, the cells have been isolated from a diseased or healthy subject prior to genetic modification to express an IDR peptide or IDR-fusion peptide, such as a CAR-IDR.

The term “Pharmaceutically acceptable carrier” describes a pharmaceutically acceptable material, composition, or vehicle that is involved in carrying or transporting a compound of interest from one tissue, organ, or portion of the body to another tissue, organ, or portion of the body. For example, in some forms the carrier is a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In some embodiments, pharmaceutical compositions include buffers such as neutral buffered saline, phosphate buffered saline and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. The pharmaceutical compositions can be formulated for delivery via any route of administration. The term “Route of administration” can refer to any administration pathway known in the art, including but not limited to aerosol, nasal, oral, intravenous, intramuscular, intraperitoneal, inhalation, transmucosal, transdermal, parenteral, implantable pump, continuous infusion, topical application, capsules and/or injections. The pharmaceutical compositions are preferably formulated for intravenous administration.

Typically, the disclosed pharmaceutical compositions are administered in a manner appropriate to a disease to be treated (or prevented). The quantity and frequency of administration is typically determined by such factors as the condition of the patient, and the type and severity of the patient's disease, although appropriate dosages can be determined by clinical trials.

The disclosed pharmaceutical compositions can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### III. Methods

Methods of using the disclosed compositions including, but not limited to, IDR proteins and IDR-fusion proteins are provided.

In some embodiments, the methods enhance the efficacy of cell receptor-mediated functions are provided. In particular embodiments, the methods provide enhanced anti-tumor activity through administration of CAR-T cells including CAR-IDR fusion peptides. It has been established that the antigen sensitivity of CAR can be enhanced by the presence of one or more IDRs, this can be utilized to develop a universal approach to reprogram T cell function. The features of the IDR were developed into a simple yet versatile approach to enhance Chimeric Antigen Receptor (CAR)-T cell function. Synthetic IDRs fused to the C-terminus of the CARs were engineered to promotes condensation of the CAR receptor (CAR-IDRs), which resulted in higher CAR-T activation and killing of low antigen-expressing cells.

As set forth in the Examples below, CAR-IDR cells showed significantly enhanced cytotoxicity towards tumor antigen-expressing cells in vitro; and CAR-IDR cells have the most durable in vivo anti-tumor effect, substantially more potent than control CAR-T cells. Together, these data here provide a distinct approach to engineer CAR-T cells with functional reprogramming via addition of one or more IDRs. The enhanced CAR-T function is (i) independent of CAR types and (ii) can be applied to a broad range of cell therapy. Furthermore, based on the modular feature of CAR, IDR can be combined with other engineering strategies for improving antigen sensitivity, including selecting specific transmembrane or co-signaling domains (Majzner, R. G. et al., Cancer Discov, doi:10.1158/2159-8290. CD-19-0945 (2020); Heitzeneder, S. et al., Cancer Cell 40, 53-69 e59, (2022); Priceman, S. J. et al., Oncoimmunology 7, e1380764, (2018)), adding new signaling binding motifs (Salter, A. I. et al., Sci Signal 14, (2021)), replacing the intracellular part with that from TCR (HIT) (Mansilla-Soto, J. et al., Nat Med 28, 345-352, (2022)), dual targeting by CAR together with chimeric costimulatory receptors (Katsarou, A. et al., Sci Transl Med 13, eabh1962, (2021)), implementing other signaling pathways (Wilkens, A. B. et al. Blood 140, 2261-2275, (2022)), and/or host cell gene knockdown or knockout (Mirzaei, et al., Cancer Lett., 2018 Jun. 1; 423:95-104. doi: 10.1016/j.canlet.2018.03.010. Epub 2018 Mar. 12; “BATF Knockout in CAR T Cells Prevents Exhaustion in Solid Tumors” Cancer Discov (2022) 12 (12): OF9, doi.org/10.1158/2159-8290. CD-RW2022-188, Kamali, et al., BMC Biotechnol 21, 9 (2021). doi.org/10.1186/s12896-020-00665-4, Dai, et al., Nat Methods,. 2019 March; 16(3):247-254. doi: 10.1038/s41592-019-0329-7. Epub 2019 Feb. 25.) to increase activity, prevent exhaustion, reduce toxicity, or achieve more than additive effects.

### A. Methods of Treatment

Methods of treatment including cells and other therapeutic agents including IDR polypeptides and IDR-fusion polypeptides are described. In preferred embodiments, the methods include Adoptive Cell Therapy (ACT) employing T cells expressing recombinant CAR-IDR fusion proteins. The CAR T cells including CAR-IDR fusion proteins have enhanced anti-tumor activity. For example, the CAR T cells including CAR-IDR fusion proteins show enhanced CAR-T activation and killing of cancer cells, particularly in killing of low antigen-expressing cells, as compared to CAR T cells including CAR proteins in the absence of IDR.

An exemplary method involves treating a subject (e.g., a human) having a disease, disorder, or condition by administering to the subject an effective amount of a pharmaceutical composition including genetically modified cells including IDR polypeptides and/or IDR-fusion polypeptides. In some embodiments, the methods administer genetically manipulated T cells engineered to express recombinant CAR-IDR fusion proteins to a subject (e.g., a human) having a disease, disorder, or condition in an amount effective to treat the disease, disorder, or condition. For example, in some embodiments, the methods treat a disease or disorder associated with an elevated expression or specific expression of an antigen by administering to the subject an effective amount of a pharmaceutical composition including cells modified to express recombinant CAR-IDR fusion proteins. In some embodiments, the methods treat a subject having a disease, disorder, or condition associated with an elevated expression or specific expression of an antigen by administering to the subject an effective amount of a pharmaceutical composition including T cells modified to contain a CAR-IDR that targets the antigen.

Methods of using IDR-fusion proteins, such as a CAR-IDR, to treat a disease or disorder by are provided. Typically, the methods enhance ACT, for example, by providing CAR-IDR-bearing T cells with enhanced therapeutic efficacy in vivo. The CAR-IDR-T cells have prolonged survival/serum residency time in vivo relative to CAR-T cells lacking the IDR fusion domain. Methods of treating a subject having a disease, disorder, or condition including administering to the subject an effective amount of a pharmaceutical composition including live, viable cells engineered to express a CAR-IDR and/or another IDR-fusion protein are provided. In some embodiments, when the methods treat a subject having a disease, disorder, or condition associated with an elevated expression or specific expression of an antigen, the methods include administering to the subject an effective amount of a T cell modified to express a CAR-IDR that targets the antigen. For example, in some forms, the methods treat a subject having a disease, disorder, or condition by administering to the subject an effective amount of a pharmaceutical composition having a genetically modified cell, where the cell is modified by introducing to the cell:
(i) a vector, optionally including a transposon encoding a CAR-IDR and/or other IDR-fusion protein; and
(ii) causing the CAR-IDR and/or other IDR-fusion protein to be expressed by the cell. The cell can have been isolated from the subject having the disease, disorder, or condition, or from a healthy donor, prior to genetic modification.

In additional or alternative to ACT, CAR-IDR-expressing cells can be created in vivo in a subject in need thereof by introducing an effective amount of nucleic acids (e.g., RNA, viral vectors, etc.) encoding the CAR-IDR into the subject. In some embodiments, the constructs are targeted to the tumor microenvironment using, e.g., targeted delivery vehicles. See, e.g., Xin, et al., Front. Oncol., 10 Feb. 2022, Sec. Cancer Immunity and Immunotherapy, Volume 12-20221 doi.org/10.3389/fonc.2022.809754.

### 1. Diseases to be Treated

Methods of treating diseases and/or disorders in a subject in need thereof are provided. The subject to be treated can have a disease, disorder, or condition such as but not limited to, cancer, an immune system disorder such autoimmune disease, an inflammatory disease, a neuronal disorder, HIV/AIDS, diabetes, a cardiovascular disease, an infectious disease, or combinations thereof. The disease, disorder, or condition can be associated with an elevated expression or specific expression of an antigen.

a. Cancer

In some embodiments, the methods treat or prevent cancer. In some forms, the methods treat or prevent cancer or other proliferative disease or disorder in a subject identified as having, or at risk of having cancer or other proliferative disease or disorder. Cancer is a disease of genetic instability, allowing a cancer cell to acquire the hallmarks proposed by Hanahan and Weinberg, including (i) self-sufficiency in growth signals; (ii) insensitivity to anti-growth signals; (iii) evading apoptosis; (iv) sustained angiogenesis; (v) tissue invasion and metastasis; (vi) limitless replicative potential; (vii) reprogramming of energy metabolism; and (viii) evading immune destruction (Cell., 144:646-674, (2011)).

Tumors, which can be treated in accordance with the disclosed methods, are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The disclosed compositions and methods of treatment thereof are generally suited for treatment of carcinomas, sarcomas, lymphomas and leukemias. The described compositions and methods are useful for treating, or alleviating subjects having benign or malignant tumors by delaying or inhibiting the growth/proliferation or viability of tumor cells in a subject, reducing the number, growth, or size of tumors, inhibiting, or reducing metastasis of the tumor, and/or inhibiting or reducing symptoms associated with tumor development or growth.

The types of cancer that can be treated with the provided compositions and methods include, but are not limited to, cancers such as vascular cancer such as multiple myeloma, adenocarcinomas, and sarcomas, of bone, bladder, brain, breast, cervical, colorectal, esophageal, kidney, liver, lung, nasopharyngeal, pancreatic, prostate, skin, stomach, and uterine. In some forms, the compositions are used to treat multiple cancer types concurrently. The compositions can also be used to treat metastases or tumors at multiple locations.

Exemplary tumor cells include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytoma and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinoma including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoid cystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, and carcinosarcoma. For a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

b. Immune System Disorders

In some embodiments, the methods administer modified T cells including CAR-IDR and/or other IDR-fusion protein(s) to treat or prevent one or more immune system disorders, including autoimmune diseases.

Under certain circumstances, the ability of the immune system to distinguish self from foreign antigens can be misdirected against healthy tissues, resulting in the undesirable attack and destruction of normal host cells (i.e., autoimmune diseases). Autoimmune diseases include over 100 types of diseases, with varied etiology and prognoses based on factors such as the affected region, the age of onset, response to the therapeutic agents and clinical manifestation may vary among different people (Muhammad, et al., Chimeric Antigen Receptor Based Therapy as a Potential Approach in Autoimmune Diseases: How Close Are We to the Treatment, Frontiers in Immunology, 11 (2020)).

Auto-antibody-secreting B lymphocytes and self-reactive T-lymphocytes play a key role in the development of autoimmune diseases. Based on the extent of tissue damage, autoimmunity is classified into two general categories, including organ-specific and systemic autoimmune. The former involves a specific area of the body such as type I diabetes (TID), multiple sclerosis (MS), rheumatoid arthritis (RA), inflammatory bowel diseases (IBDs), and myasthenia gravis (MG), while the latter affects multiple regions of the body, causing systemic lupus erythematosus (SLE) and Sjögren's syndrome (SS). Therefore, in some forms, the methods treat or prevent one or more organ-specific autoimmune diseases in a subject. In other forms, the methods treat or prevent one or more systemic autoimmune diseases in a subject.

In some forms, the methods reduce or prevent one or more physiological processes associated with the development or progression of autoimmune disease in a subject. For example, in some forms, the methods reduce or prevent one or more of epitope spreading, for example, where infections alter the primary epitope into the secondary epitope or form several neoepitopes on antigen-presenting cells; bystander activation or pre-primed autoreactive T cell activation in a T cell receptor (TCR)-independent manner; persistent virus infection, or the constant presence of viral antigens that prompt immune responses; or immunological cross-reactivity between a host and pathogen, for example, due to shared immunologic epitopes or sequence similarities. Non-limiting examples of immune system disorders that can be treated or prevented by the methods include 22q11.2 deletion syndrome, Achondroplasia and severe combined immunodeficiency, Adenosine Deaminase 2 deficiency, Adenosine deaminase deficiency, Adult-onset immunodeficiency with anti-interferon-gamma autoantibodies, Agammaglobulinemia, non-Bruton type, Aicardi-Goutieres syndrome, Aicardi-Goutieres syndrome type 5, Allergic bronchopulmonary aspergillosis, Alopecia, Alopecia totalis, Alopecia universalis, Amyloidosis AA, Amyloidosis familial visceral, Ataxia telangiectasia, Autoimmune lymphoproliferative syndrome, Autoimmune lymphoproliferative syndrome due to CTLA4 haploinsuffiency, Autoimmune polyglandular syndrome type 1, Autosomal dominant hyper IgE syndrome, Autosomal recessive early-onset inflammatory bowel disease, Autosomal recessive hyper IgE syndrome, Bare lymphocyte syndrome 2, Barth syndrome, Blau syndrome, Bloom syndrome, Bronchiolitis obliterans, C1q deficiency, Candidiasis familial chronic mucocutaneous, autosomal recessive, Cartilage-hair hypoplasia, CHARGE syndrome, Chediak-Higashi syndrome, Cherubism, Chronic atypical neutrophilic dermatosis with lipodystrophy and elevated temperature, Chronic graft versus host disease, Chronic granulomatous disease, Chronic Infantile Neurological Cutaneous Articular syndrome, Chronic mucocutaneous candidiasis (CMC), Cohen syndrome, Combined immunodeficiency with skin granulomas, Common variable immunodeficiency, Complement component 2 deficiency, Complement component 8 deficiency type 1, Complement component 8 deficiency type 2, Congenital pulmonary alveolar proteinosis, Cryoglobulinemia, Cutaneous mastocytoma, Cyclic neutropenia, Deficiency of interleukin-1 receptor antagonist, Dendritic cell, monocyte, B lymphocyte, and natural killer lymphocyte deficiency, Dyskeratosis congenital, Dyskeratosis congenita autosomal dominant, Dyskeratosis congenita autosomal recessive, Dyskeratosis congenita X-linked, Epidermodysplasia verruciformis, Familial amyloidosis, Finnish type, Familial cold autoinflammatory syndrome, Familial Mediterranean fever, Familial mixed cryoglobulinemia, Felty's syndrome, Glycogen storage disease type 1B, Griscelli syndrome type 2, Hashimoto encephalopathy, Hashimoto's syndrome, Hemophagocytic lymphohistiocytosis, Hennekam syndrome, Hepatic venoocclusive disease with immunodeficiency, Hereditary folate malabsorption, Hermansky Pudlak syndrome 2, Herpes simplex encephalitis, Hoyeraal Hreidarsson syndrome, Hyper IgE syndrome, Hyper-IgD syndrome, ICF syndrome, Idiopathic acute eosinophilic pneumonia, Idiopathic CD4 positive T-lymphocytopenia, IL12RB1 deficiency, Immune defect due to absence of thymus, Immune dysfunction with T-cell inactivation due to calcium entry defect 1, Immune dysfunction with T-cell inactivation due to calcium entry defect 2, Immunodeficiency with hyper IgM type 1, Immunodeficiency with hyper IgM type 2, Immunodeficiency with hyper IgM type 3, Immunodeficiency with hyper IgM type 4, Immunodeficiency with hyper IgM type 5, Immunodeficiency with thymoma, Immunodeficiency without anhidrotic ectodermal dysplasia, Immunodysregulation, polyendocrinopathy and enteropathy X-linked, Immunoglobulin A deficiency 2, Intestinal atresia multiple, IRAK-4 deficiency, Isolated growth hormone deficiency type 3, Kawasaki disease, Large granular lymphocyte leukemia, Leukocyte adhesion deficiency type 1, LRBA deficiency, Lupus, Lymphocytic hypophysitis, Majeed syndrome, Melkersson-Rosenthal syndrome, MHC class 1 deficiency, Muckle-Wells syndrome, Multifocal fibrosclerosis, Multiple sclerosis, MYD88 deficiency, Neonatal systemic lupus erythematosus, Netherton syndrome, Neutrophil-specific granule deficiency, Nijmegen breakage syndrome, Omenn syndrome, Osteopetrosis autosomal recessive 7, Palindromic rheumatism, Papillon Lefevre syndrome, Partial androgen insensitivity syndrome, PASLI disease, Pearson syndrome, Pediatric multiple sclerosis, Periodic fever, aphthous stomatitis, pharyngitis and adenitis, PGM3-CDG, Poikiloderma with neutropenia, Pruritic urticarial papules plaques of pregnancy, Purine nucleoside phosphorylase deficiency, Pyogenic arthritis, pyoderma gangrenosum and acne, Relapsing polychondritis, Reticular dysgenesis, Sarcoidosis, Say Barber Miller syndrome, Schimke immunoosseous dysplasia, Schnitzler syndrome, Selective IgA deficiency, Selective IgM deficiency, Severe combined immunodeficiency, Severe combined immunodeficiency due to complete RAG1/2 deficiency, Severe combined immunodeficiency with sensitivity to ionizing radiation, Severe combined immunodeficiency, Severe congenital neutropenia autosomal recessive 3, Severe congenital neutropenia X-linked, Shwachman-Diamond syndrome, Singleton-Merten syndrome, SLC35C1-CDG (CDG-IIc), Specific antibody deficiency, Spondyloenchondrodysplasia, Stevens-Johnson syndrome, T-cell immunodeficiency, congenital alopecia and nail dystrophy, TARP syndrome, Trichohepatoenteric syndrome, Tumor necrosis factor receptor-associated periodic syndrome, Twin to twin transfusion syndrome, Vici syndrome, WHIM syndrome, Wiskott Aldrich syndrome, Woods Black Norbury syndrome, X-linked agammaglobulinemia, X-linked lymphoproliferative syndrome, X-linked lymphoproliferative syndrome 1, X-linked lymphoproliferative syndrome 2, X-linked magnesium deficiency with Epstein-Barr virus infection and neoplasia, X-linked severe combined immunodeficiency, and ZAP-70 deficiency.

The disclosed compositions and methods can also be used to treat autoimmune diseases or disorders. Exemplary autoimmune diseases or disorders, which are not mutually exclusive with the immune system disorders described above, include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Bald disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

c. Other Disease or Disorders

In some forms the methods administer modified T cells including CAR-IDR and/or other IDR-fusion protein(s) to treat one or more additional disease or disorder in a subject in need thereof. For example, in some forms the methods treat one or more genetic disease or disorders in a subject, such as a hereditary genetic disease or disorder, or a somatic genetic disease or disorder in a subject.

Any of the methods can include treating a subject having an underlying disease or disorder. For example, in some forms, the methods treat a disease or disorder, such as a cancer or auto-immune disease in a patient having another disease or disorder, such as diabetes, a bacterial infection (e.g., Tuberculosis), viral infection (e.g., Hepatitis, HIV, HPV infection, etc.), or a drug-associated disease or disorder. In some forms, the methods treat an immunocompromised subject. In some forms, the methods treat a subject having a disease of the kidney, liver, heart, lung, brain, bladder, reproductive system, bowel/intestines, stomach, bones, or skin.

### B. Effective Amounts

In some forms the methods administer modified T cells including CAR-IDR and/or other IDR-fusion protein(s) in an effective amount. The effective amount or therapeutically effective amount of a pharmaceutical compositions including modified cells, such as therapeutic T cells, can be a dosage sufficient to treat, inhibit, or alleviate one or more symptoms of a disease or disorder, such as a cancer or autoimmune disease, or to otherwise provide a desired pharmacologic and/or physiologic effect, for example, reducing, inhibiting, or reversing one or more of the underlying pathophysiological mechanisms underlying a disease or disorder, such as cancer or autoimmune disease.

In some forms, when administration of the pharmaceutical compositions including modified cells, such as therapeutic T cells, including CAR-IDR and/or other IDR-fusion protein(s) elicits an anti-cancer response, the amount administered can be expressed as the amount effective to achieve a desired anti-cancer effect in the recipient. For example, in some forms, the amount of the pharmaceutical compositions including modified cells, such as therapeutic T cells, is effective to inhibit the viability or proliferation of cancer cells in the recipient. In some forms, the amount of the pharmaceutical composition including modified cells, such as therapeutic T cells, is effective to reduce the tumor burden in the recipient, or reduce the total number of cancer cells, and combinations thereof. In other forms, the amount of the pharmaceutical compositions including modified cells, such as therapeutic T cells, is effective to reduce one or more symptoms or signs of cancer in a cancer patient, or signs of an autoimmune disease in a patient having an autoimmune disease or disorder. Signs of cancer can include cancer markers, such as PSMA levels in the blood of a patient.

The effective amount of the pharmaceutical compositions including modified cells, such as therapeutic T cells, that is required will vary from subject to subject, depending on the species, age, weight and general condition of the subject, the severity of the disorder being treated, and its mode of administration. Thus, it is not possible to specify an exact amount for every pharmaceutical composition. However, an appropriate amount can be determined by one of ordinary skill in the art using only routine experimentation given the teachings herein. For example, effective dosages and schedules for administering the pharmaceutical compositions including therapeutic T cells can be determined empirically, and making such determinations is within the skill in the art. In some forms, the dosage ranges for the administration of the compositions including therapeutic T cells are those large enough to effect reduction in cancer cell proliferation or viability, or to reduce tumor burden for example.

The dosage should not be so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the composition including therapeutic T cells used for treatment can increase or decrease over the course of a particular treatment. Changes in dosage can result and become apparent from the results of diagnostic assays.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions and can generally be estimated based on EC₅₀s found to be effective in in vitro and in vivo animal models.

It can generally be stated that a pharmaceutical composition containing CAR-IDR T cells described herein can be administered at a dosage of 10⁴ to 10⁹ cells/kg body weight, preferably 10⁵ to 10⁷ cells/kg body weight, including all integer values within those ranges. In some forms, patients can be treated by infusing a disclosed pharmaceutical composition containing CAR-IDR expressing cells (e.g., T cells) in the range of about 10⁴ to 10¹² or more cells per square meter of body surface (cells/m).

The infusion can be repeated as often and as many times as the patient can tolerate until the desired response is achieved. CAR-IDR T cell compositions can also be administered once or multiple times at these dosages. The cells can be administered by using infusion techniques that are commonly known in immunotherapy (see, e.g., Rosenberg et al, New Eng. J. of Med. 319:1676, 1988). The optimal dosage and treatment regime for a particular patient can readily be determined by one skilled in the art of medicine by monitoring the patient for signs of disease and adjusting the treatment accordingly. In some forms, the unit dosage is in a unit dosage form for intravenous injection. In some forms, the unit dosage is in a unit dosage form for oral administration. In some forms, the unit dosage is in a unit dosage form for inhalation. In some forms, the unit dosage is in a unit dosage form for intra-tumoral injection.

Treatment can be continued for an amount of time sufficient to achieve one or more desired therapeutic goals, for example, a reduction of the amount of cancer cells relative to the start of treatment, or complete absence of cancer cells in the recipient. Treatment can be continued for a desired period of time, and the progression of treatment can be monitored using any means known for monitoring the progression of anti-cancer treatment in a patient. In some forms, administration is carried out every day of treatment, or every week, or every fraction of a week. In some forms, treatment regimens are carried out over the course of up to two, three, four or five days, weeks, or months, or for up to 6 months, or for more than 6 months, for example, up to one year, two years, three years, or up to five years.

The efficacy of administration of a particular dose of the pharmaceutical compositions including modified cells, such as therapeutic T cells, according to the methods described herein can be determined by evaluating the aspects of the medical history, signs, symptoms, and objective laboratory tests that are known to be useful in evaluating the status of a subject in need for the treatment of cancer or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious. In some forms, efficacy is assessed as a measure of the reduction in tumor volume and/or tumor mass at a specific time point (e.g., 1-5 days, weeks, or months) following treatment.

### C. Modes of Administration

In some embodiments the methods administer modified T cells including CAR-IDR and/or other IDR-fusion protein(s) in combination with a pharmaceutically acceptable carrier. The compositions described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, PA, which discloses typical carriers and conventional methods of preparing pharmaceutical compositions that can be used in conjunction with the preparation of formulations of the therapeutics described herein and which is incorporated by reference herein. These most typically would be standard carriers for administration of compositions to humans. In one aspect, for humans and non-humans, these include solutions such as sterile water, saline, and buffered solutions at physiological pH. Other therapeutics can be administered according to standard procedures used by those skilled in the art.

The pharmaceutical compositions including modified cells, such as therapeutic T cells, described herein can include, but are not limited to, carriers, thickeners, diluents, buffers, preservatives, surface active agents and the like in addition to the therapeutic(s) of choice.

Pharmaceutical compositions containing one or more modified cells, such as therapeutic T cells including CAR-IDR and/or other IDR-fusion protein(s), and optionally one or more additional therapeutic agents can be administered to the subject in a number of ways depending on whether local or systemic treatment is desired, and on the area to be treated. Thus, for example, a pharmaceutical composition including modified cells, such as therapeutic T cells, can be administered as an intravenous infusion, or directly injected into a specific site, for example, into or surrounding a tumor. Moreover, a pharmaceutical composition can be administered to a subject as an ophthalmic solution and/or ointment to the surface of the eye, vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. In some forms, the compositions are administered directly into a tumor or tissue, e.g., stereotactically.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant including a porous, non-porous, or gelatinous material).

Preparations for parenteral administration include sterile aqueous or non-aqueous solutions, suspensions, and emulsions which can also contain buffers, diluents, and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, antioxidants, chelating agents, and inert gases and the like.

Administration of the pharmaceutical compositions containing one or more genetically modified cells (e.g., CAR-IDR T cells) can be localized (i.e., to a particular region, physiological system, tissue, organ, or cell type) or systemic.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### D. Combination Therapy

In some embodiments the methods administer modified T cells including CAR-IDR and/or other IDR-fusion protein(s) in combination with other therapeutic agents or treatment modalities. Any of the disclosed pharmaceutical compositions including modified cells, such as therapeutic T cells (e.g., containing a population of CAR-IDR T-cells), can be used alone, or in combination with other therapeutic agents or treatment modalities, for example, chemotherapy or stem-cell transplantation. As used herein, “combination” or “combined” refer to either concomitant, simultaneous, or sequential administration of the therapeutics.

In some forms, the pharmaceutical compositions and other therapeutic agents are administered separately through the same route of administration. In other forms, the pharmaceutical compositions and other therapeutic agents are administered separately through different routes of administration. The combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one agent is given first followed by the second).

Examples of preferred additional therapeutic agents include other conventional therapies known in the art for treating the desired disease, disorder, or condition. In some forms, the therapeutic agent is one or more other targeted therapies (e.g., a targeted cancer therapy) and/or immune-checkpoint blockage agents (e.g., anti-CTLA-4, anti-PD1, and/or anti-PDL1 agents such as antibodies).

The compositions and methods described herein may be used as a first therapy, second therapy, third therapy, or combination therapy with other types of therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

The disclosed pharmaceutical compositions and/or other therapeutic agents, procedures or modalities can be administered during periods of active disease, or during a period of remission or less active disease. The pharmaceutical compositions can be administered before the additional treatment, concurrently with the treatment, post-treatment, or during remission of the disease or disorder. When administered in combination, the disclosed pharmaceutical compositions, and the additional therapeutic agents (e.g., second or third agent), or all, can be administered in an amount or dose that is higher, lower or the same than the amount or dosage of each agent used individually, e.g., as a monotherapy. In certain forms, the administered amount or dosage of the disclosed pharmaceutical composition, the additional therapeutic agent (e.g., second or third agent), or all, is lower (e.g., at least 20%, at least 30%, at least 40%, or at least 50%) than the amount or dosage of each agent used individually, e.g., as a monotherapy (e.g., required to achieve the same therapeutic effect).

### 1. Additional Anti-Cancer Agents

In some embodiments, the methods administer one or more additional anti-cancer agents to a subject.

In the context of cancer, targeted therapies are therapeutic agents that block the growth and spread of cancer by interfering with specific molecules (“molecular targets”) that are involved in the growth, progression, and spread of cancer. Many different targeted therapies have been approved for use in cancer treatment. These therapies include hormone therapies, signal transduction inhibitors, gene expression modulators, apoptosis inducers, angiogenesis inhibitors, immunotherapies, and toxin delivery molecules. Numerous antineoplastic drugs can be used in combination with the disclosed pharmaceutical compositions. In some forms, the additional therapeutic agent is a chemotherapeutic or antineoplastic drug. The majority of chemotherapeutic drugs can be divided into alkylating agents, antimetabolites, anthracyclines, plant alkaloids, topoisomerase inhibitors, monoclonal antibodies, and other anti-tumor agents.

### 2. Additional Therapeutic Agents Against Autoimmune Diseases

In some embodiments, the methods also include administering one or more conventional therapies for autoimmune diseases to the subject.

Exemplary therapies for autoimmune diseases include immunosuppressive agents, such as steroids or cytostatic drugs, analgesics, non-steroidal anti-inflammatory drugs, glucocorticoids, immunosuppressive and immunomodulatory agents, such as methotrexate, leflunomide, hydroxychloroquine, and sulfasalazine. In some forms, the methods administer one or more disease-modifying antirheumatic drugs (DMARDs). In some forms, the methods administer one or more biologic agents for localized treatment (i.e., agents that do not affect the entire immune system), such as TNF-α inhibitors, belimumab and rituximab depleting B cells, T-cell co-stimulation blocker, anti-interleukin 6 (IL-6), anti-IL-1, and protein kinase inhibitors. In other forms, the methods also administer one or more monoclonal antibodies (mAbs), such as anti-TNFα, anti-CD19, anti-CD20, anti-CD22, and anti-IL6R, or other mAbs that target multiple B cell subtypes, and other aberrant cells in autoimmune diseases.

### IV. Kits

The compositions, reagents, and other materials for cellular genomic engineering can be packaged together in any suitable combination as a kit useful for performing, or aiding in the performance of, the methods. It is useful if the components in a given kit are designed and adapted for use together in the method. For example, kits with one or more compositions for administration to a subject, may include a pre-measured dosage of the composition in a sterile needle, ampule, tube, container, or other suitable vessel. The kits may include instructions for dosages and dosing regimens.

Provided are kits containing a IDR-fusion peptide (e.g., CAR-IDR) within a vector (e.g., a viral vector) and/or mRNA encoding the IDR-fusion peptide (e.g., CAR-IDR), and instructional material for use thereof. In preferred forms, the kit includes a plurality of vectors, where each vector independently contains a IDR-fusion peptide (e.g., CAR-IDR) for insertion into a host cell genome, such as a CAR expression cassette. In some forms, the kit contains a population of cells (e.g., T cells) collectively containing the IDR-fusion peptide (e.g., CAR-IDR). The instructional material can include a publication, a recording, a diagram, or any other medium of expression which can be used to communicate the usefulness of the compositions and methods of the kit. For example, the instructional material may provide instructions for methods using the kit components, such as performing transfections, transductions, infections, and conducting screens. In some forms, kits include a transposon that includes a promoter and/or polyadenylation signal operationally linked to a reporter gene and/or a CAR; in some forms, the kit includes a transposon including a CAR that is specific for an antigen selected from a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof; for example, in some embodiments the CAR targets one or more antigens selected from the group including AFP, AKAP 4, ALK, Androgen receptor, B7H3, BCMA, Bcr Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD2, GD3, GloboH, GM3, gp100, GPC3, HER 2/neu, HER2, HMWMAA, HPV E6/E7, hTERT, Idiotype, IL12, IL13RA2, IM19, IX, LCK, Legumain, IgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR β, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, and XAGE.

In some embodiments, the kit includes a cell or vector including a CAR-IDR polypeptide, or other IDR-fusion peptide or nucleic acid encoding a CAR-IDR, or other IDR-fusion peptide. In some embodiments, the CAR-IDR is specific for an antigen that is selected from a cancer antigen selected from 4 1 BB, 5T4, adenocarcinoma antigen, alpha fetoprotein, BAFF, B lymphoma cell, C242 antigen, CA 125, carbonic anhydrase 9 (CA IX), C MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD2, GD3 ganglioside, glycoprotein 75, GPNMB, HER2/neu, HGF, human scatter factor receptor kinase, IGF 1 receptor, IGF I, IgGI, L1 CAM, IL 13, IL 6, insulin-like growth factor I receptor, integrin α5β1, integrin αvβ3, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF β, TRAIL R1, TRAIL R2, tumor antigen CTAA16.88, VEGF A, VEGFR 1, VEGFR2, and vimentin; in some forms, the CAR is bispecific or multivalent; in some forms, the CAR is anti-CD19, anti-CD22, and/or anti-HER2.

In exemplary embodiments, the kits include a nucleic acid and/or a vector expressing or encoding the CAR-IDR and/or cells. Exemplary cells include a T cell, hematopoietic stem cell (HSC), macrophage, natural killer cell (NK), or dendritic cell (DC). In some embodiments, the T cell is a CD8+ T cell selected from effector T cells, memory T cells, central memory T cells, and effector memory T cells. In some embodiments, the T cell is a CD4+ T cell selected from Th1 cells, Th2 cells, Th17 cells, and Treg cells.

The present invention can be further understood by the following numbered paragraphs.

1. A polypeptide including
(a) an amino acid sequence of an intrinsically disordered region (IDR) of a protein, optionally including between 50 and 500 amino acids, or a functional fragment or variant thereof; and
(b) an amino acid sequence that is heterologous to the IDR.

2. The polypeptide of paragraph 1, wherein the amino acid sequence of the IDR includes any one of SEQ ID NOs:1-6, or a functional variant thereof having an amino acid sequence at least 70%, 75%, 80%, 85%, 90%, 95%, or 99% identical to any one of SEQ ID NOs:1-6.

3. The polypeptide of paragraphs 1 or 2, wherein the amino acid sequence of the IDR includes at least 70% and less than 100% sequence identity to any one of SEQ ID NOs:1-6, or a homologue thereof, or functional fragment of the foregoing.

4. The polypeptide of any one of paragraphs 1-3, wherein the heterologous sequence includes one or more of a chimeric antigen receptor (CAR), protein transduction domain, fusogenic polypeptide, targeting signal, expression, and/or purification tag.

5. The polypeptide of paragraph 4, wherein the heterologous sequence includes a chimeric antigen receptor (CAR), and wherein the amino acid sequence of the IDR is present within the extracellular or intracellular region of the CAR.

6. The polypeptide of paragraph 4 or 5, wherein the heterologous sequence includes a chimeric antigen receptor (CAR), and wherein the amino acid sequence of the IDR is contiguous with the carboxyl terminus or amino terminus of the CAR, or inserted between two domains of the CAR, optionally wherein the polypeptide includes a structure according to:
(i) N-[CAR]-[IDR]_{Z}-C, where “N” and “C” refer to the amino (NH₂) and Carboxyl (COOH) termini, respectively, “CAR” refers to a single contiguous CAR fusion protein, “IDR” refers to an IDR and “Z” is an integer between one and four, inclusive; or
(ii) N-[CAR part 1]-[IDR]_{Z}-[CAR part 2], where “N” and “C” refer to the amino (NH₂) and Carboxyl (COOH) termini, respectively, “IDR” refers to an IDR and “Z” is an integer between one and four, inclusive, “CAR-part 1” includes one or more traditional CAR domains and “CAR-part 2” includes one or more traditional CAR domains such that without the intervening [IDR]_{Z} domain, CAR-part 1 and CAR-part 2 form a single contiguous CAR fusion protein.

7. The polypeptide of any one of paragraphs 4-6, wherein the heterologous sequence includes a chimeric antigen receptor (CAR) including a transmembrane domain of CD8a, cytosolic signaling domains or a fragment there of derived from CD28, 41BB, and CD3 zeta, and optionally wherein the amino acid sequence of the IDR is contiguous with the intracellular component of CD3 zeta.

8. The polypeptide of any one of paragraphs 4-7, wherein the CAR is specific for an antigen selected from the group consisting of a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

9. The polypeptide of paragraph 8, wherein the CAR targets one or more antigens selected from the group consisting of AFP, AKAP 4, ALK, Androgen receptor, B7H3, BCMA, Bcr Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD2, GD3, GloboH, GM3, gp100, GPC3, HER 2/neu, HER2, HMWMAA, HPV E6/E7, hTERT, Idiotype, IL12, IL13RA2, IM19, IX, LCK, Legumain, IgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR β, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, XAGE, Claudin-6, Claudin-18.2 and CD70.

10. The polypeptide of paragraph 8, wherein the antigen is a cancer antigen selected from the group consisting of 41BB, 5T4, adenocarcinoma antigen, alpha fetoprotein, BAFF, B lymphoma cell, C242 antigen, CA 125, carbonic anhydrase 9 (CA IX), C MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD2, GD3 ganglioside, glycoprotein 75, GPNMB, HER2/neu, HGF, human scatter factor receptor kinase, IGF 1 receptor, IGF I, IgG1, L1 CAM, IL 13, IL 6, insulin-like growth factor I receptor, integrin α5β1, integrin αvβ3, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF β, TRAIL R1, TRAIL R2, tumor antigen CTAA16.88, VEGF A, VEGFR 1, VEGFR2, and vimentin.

11. The polypeptide of paragraph 10, wherein the CAR is anti-CD19, anti-CD22, or anti-HER2.

12. The polypeptide of any one of paragraphs 1-11, including the polypeptide sequence of any one of SEQ ID NOs:7-27.

13. The polypeptide of any one of paragraphs 1-12, lacked a coiled-coiled domain.

14. A nucleic acid including a nucleic acid encoding the polypeptide of any one of paragraphs 1-13.

15. The nucleic acid of paragraph 14, wherein the nucleic acid is RNA or DNA.

16. The nucleic acid of paragraph 14 or 15, wherein the nucleic acid is mRNA.

17. The nucleic acid of any one of paragraphs 14-16, wherein the nucleic acid includes an expression control sequence(s).

18. The nucleic acid of any one of paragraphs 14-17, wherein the nucleic acid is, or is encoded by a vector or a transposon.

19. The nucleic acid of paragraph 18, wherein the vector is a viral vector.

20. The nucleic acid of paragraph 19, wherein the viral vector is selected from the group consisting of a lentiviral vector, an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector including a combination of any two or more of an Adeno-associated virus (AAV) vector, Herpes Simplex virus (HSV) vector, vesicular stomatitis (VSV) vector, or a human Bocavirus vector (hBoV).

21. The nucleic acid of paragraph 18, wherein the vector is a nucleic acid expression vector selected from the group consisting of a plasmid, a cosmid, and a replicon.

22. The nucleic acid of any one of paragraphs 14-21, wherein the nucleic acid includes a promotor.

23. The nucleic acid of any one of paragraphs 14-22, including one or more of a protein transduction domain, fusogenic polypeptide, or targeting signal conjugated thereto.

24. An isolated cell including the polypeptide of any one of paragraphs 1-12, or the nucleic acid of any one of paragraphs 14-23.

25. The isolated cell of paragraph 24, wherein the cell is a T cell, hematopoietic stem cell (HSC), macrophage, natural killer cell (NK), or dendritic cell (DC).

26. The isolated cell of paragraph 25, wherein the T cell is a CD8+ T cell selected from the group consisting of effector T cells, memory T cells, central memory T cells, and effector memory T cells.

27. The isolated cell of paragraph 25, wherein the T cell is a CD4+ T cell selected from the group consisting of Th1 cells, Th2 cells, Th17 cells, and Treg cells.

28. The isolated cell of any one of paragraphs 24-27, wherein the polypeptide includes
(i) an amino acid sequence encoding a CAR; and
(ii) an amino acid sequence of one or more of SEQ ID NOs:1-6, or a variant having at least 70%, 75%, 80%, 85%, 90%, 95%, or 99% identity to one or more of SEQ ID NOs:1-6.

29. The isolated cell of any one of paragraphs 24-28, wherein the polypeptide includes an amino acid sequence of one or more of SEQ ID NOs:7-27, or a variant having at least 70%, 75%, 80%, 85%, 90%, 95%, or 99% identity to one or more of SEQ ID NOs: 7-27.

30. The isolated cells of any one of paragraphs 24-29, wherein the cell has increased secretion of one or more cytotoxic factors optionally included IFNγ, Perforin, FasL, Granzyme A, and/or Granzyme B; activation of LAT and/or pathway(s) downstream thereof, optionally including SLP76 activation, ERK activation, RAS activation, and/or actin polymerization, optionally wherein LAT activation includes LAT phosphorylation; and/or reduction of one or more T cell exhaustion markers optionally TIM3 and/or LAG3 relative to a control cell, optionally wherein the control cells is the same cell type including the same heterologous sequence without the IDR sequence.

31. A population of cells including a plurality of the cells of any one of paragraphs 24-30, optionally derived by expanding the cell of any one of paragraphs 24-30.

32. A pharmaceutical composition including the population of cells of paragraph 31 and a pharmaceutically acceptable buffer, carrier, diluent, or excipient.

33. A method of treating a subject having a disease, disorder, or condition including administering to the subject an effective amount of the pharmaceutical composition of paragraph 32.

34. A method of treating a subject having a disease, disorder, or condition associated with an elevated expression or specific expression of an antigen, the method including administering to the subject an effective amount of a cell of any one of paragraphs 27-30, wherein the CAR targets the antigen.

35. The method of any one of paragraphs 33-34, wherein the cell is isolated from the subject having the disease, disorder, or condition prior to the introduction to the cell.

36. The method of any one of paragraphs 33-34, wherein the cell is isolated from a healthy donor.

37. The method of any one of paragraphs 33-36, wherein the subject is a human.

38. The method of any one of paragraphs 33-37, wherein the subject has cancer.

39. A method of introducing an IDR fusion peptide into a cell, the method including introducing to the cell:
(i) a vector or transposon or mRNA encoding a polypeptide of any one of paragraphs 1-13; and
(ii) causing the polypeptide to be expressed in the cell.

40. A chimeric antigen receptor (CAR) including a polypeptide of an intrinsically disordered region (IDR) (CAR-IDR), wherein the CAR-IDR targets CD19 and includes the amino acid sequence of any one of SEQ ID NOs:7-18.

41. The chimeric antigen receptor (CAR) of paragraph 40, wherein the CAR-IDR has increased activity in condensation of CAR and/or activation of T cells expressing the CAR-IDR compared to a control, wherein the control has the same CAR targeting CD19 but without fusion to an IDR including the amino acid sequence of SEQ ID NO:19.

42. A chimeric antigen receptor (CAR) including a polypeptide of an intrinsically disordered region (IDR) (CAR-IDR), wherein the CAR-IDR targets CD22 and includes the amino acid sequence of any one of SEQ ID NOs: 20-22.

43. The chimeric antigen receptor (CAR) of paragraph 42, wherein the CAR-IDR has increased activity in condensation of CAR and/or activation of T cells expressing the CAR-IDR compared to a control, wherein the control has the same CAR targeting CD22 but without fusion to an IDR including the amino acid sequence of SEQ ID NO:23.

44. A chimeric antigen receptor (CAR) including a polypeptide of an intrinsically disordered region (IDR) (CAR-IDR), wherein the CAR-IDR targets HER2 and includes the amino acid sequence of any one of SEQ ID NOs:24-26.

45. The chimeric antigen receptor (CAR) of paragraph 44, wherein the CAR-IDR has increased activity in condensation of CAR and/or activation of T cells expressing the CAR-IDR compared to a control, wherein the control has the same CAR targeting HER2 but without fusion to an IDR including the amino acid sequence of SEQ ID NO:27.

46. A nucleic acid, including a nucleic acid sequence encoding the chimeric antigen receptor of any one of paragraphs 40-45.

47. The nucleic acid of paragraph 46, wherein the nucleic acid is a vector or a transposon.

48. An isolated cell including the CAR of any one of paragraphs 40-45, or the nucleic acid of any one of paragraphs 46-47.

49. A population of cells derived by expanding the cell of paragraph 48.

50. A pharmaceutical composition including the population of cells of paragraph 49 and a pharmaceutically acceptable buffer, carrier, diluent, or excipient.

51. A method of treating a subject having a disease, disorder, or condition including administering to the subject an effective amount of the pharmaceutical composition of paragraph 50.

52. The method of paragraph 51, wherein the disease is a cancer.

53. A composition or method as disclosed herein including in the text and drawings.

The present invention will be further understood by reference to the following non-limiting examples.

### EXAMPLES

Previous studies indicated that CARs insufficiently induce membrane-proximal signaling including ZAP70 activation and LAT phosphorylation, and LAT is weakly engaged in the CAR pathway (Dong et al., 2020; Gudipati et al., 2020; Salter et al., 2021). LAT enhances TCR signal transduction by promoting the macromolecular complex assembly and condensation of TCR signaling molecules (Houtman et al., 2006; Huang et al., 2019; Su et al., 2016; Zeng et al., 2021). These indicated an opportunity to engage condensation to improve CAR-T signaling, especially on its response to low antigen-expressing cancers.

Intrinsically disordered regions (IDRs) have the ability to form biomolecular condensates. IDRs do not typically fold into a well-defined 3D structure. Instead, they form condensates via weak inter and intra molecular interactions (Borcherds et al., 2021; Pappu et al., 2023). These condensates display unique biochemical activities through enriching and organizing effector molecules to promote cell signaling (Case et al., 2019; Xiao et al., 2022a). In the experiments below, CAR condensation was induced through constructing CAR-IDR fusion proteins. IDRs were identified, including those from FUS, EWS, and TAF15, that promote the membrane-proximal signaling, cytotoxic factor production, and killing of CAR-Ts against low antigen-expressing cancers (CD19, CD22, and HER2). Moreover, no elevated tonic signaling was observed in IDR CAR-Ts, indicating a difference in signaling outcomes between IDR-induced condensation and previously reported CAR aggregation (Chen et al., 2023; Long et al., 2015). Together, these results demonstrated that IDRs, though not originally linked to T cell signaling, can serve as a new modular motif to improve the anti-tumor effect of CAR-Ts.

### Materials and Methods

A list of reagents used in this study could be found in Tables 1-4.

| SEQ | | |
|---|---|---|
| ID | | |
| NO | Name | IDR Sequences |
| 1 | FUS | MASND YTQQATQSYGAYPTQPGQGY |
| | | SQQSSQPYGQQSYSGYSQST |
| | | DTSGYGQSSYSSYGQSQNTG |
| | | YGTQSTPQGYGSTGGYGSSQ |
| | | SSQSSYGQQSSYPGYGQQPAPSSTSGSYGSSSQSSS |
| | | YGQP QSGSYSQQPSYGGQQQSYGQ |
| | | QQSYNPPQGYGQQNQYNSSS |
| | | GGGGGGGGGGNYGQDQSSMS |
| | | SGGGSGGGYGNQDQSGGGGS GGYGQQDRG |
| 2 | EWS | GYAQTTQA YGQQSYGTYGQPTDVSYTQA |
| | | QTTATYGQTAYATSYGQPPT |
| | | GYTTPTAPQAYSQPVQGYGT |
| | | GAYDTTTATVTTTQASYAAQ |
| | | SAYGTQPAYPAYGQQPAATA |
| | | PTRPQDGNKPTETSQPQSST |
| | | GGYNQPSLGYGQSNYSYPQV |
| | | PGSYPMQPVTAPPSYPPTSY |
| | | SSTQPTSYDQSSYSQQNTYG |
| | | QPSSYGQQSSYGQQSSYGQQ |
| | | PPTSYPPQTGSYSQAPSQYSQQSSSYG |
| 3 | TAF15 | MSDSGSYG QSGGEQQSYSTYGNPGSQGY |
| | | GQASQSYSGYGQTTDSSYGQNYSGYSSYGQSYSQSY |
| | | GGYE NQKQSSYSQQPYNNQGQQQN |
| | | MESSGSQGGRAPSYDQPDYG |
| | | QQDSYDQQSGYDQHQGSYDE |
| | | QSNYDQQHDSYSQNQQSYHS |
| | | QRENYSHHTQDDRRDVSRYG |
| | | EDNRGYGGSQGG |
| 4 | NUP98 | FNKSFGTPFG GGTGGFGTTSTFGQNTGFGT |
| | | TSGGAFGTSAFGSSNNTGGL |
| | | FGNSQTKPGGLFGTSSFSQP |
| | | ATSTSTGFGFGTSTGTANTL |
| | | FGTASTGTSLFSSQNNAFAQ |
| | | NKPTGFGNFGTSTSSGGLFG |
| | | TTNTTSNPFGSTSGSLFGPS |
| | | SFTAAPTGTTIKFNPPTGTD |
| | | TMVKAGVSTNISTKHQCITA |
| | | MKEYESKSLEELRLEDYQAN |
| | | RKGPQNQVGAGTTTGLFGSS |
| | | PATSSATGLFSSSTTNSGFA |
| | | YGQNKTAFGTSTTGFGTNPG |
| | | GLFGQQNQQTTSLFSKPFGQ |
| | | ATTTQNTGFSFGNTSTIGQP |
| | | STNTMGLFGVTQASQPGGLF GTATNTSTGT |
| 5 | TDP43 | SNRQLERSGR FGGNPGGFGNQGGFGNSRGG |
| | | GAGLGNNQGSNMGGGMNFGA |
| | | FSINPAMMAAAQAALQSSWG |
| | | MMGMLASQQNQSGPSGNNQN |
| | | QGNMQREPNQAFGSGNNSYS |
| | | GSNSGAAIGWGSASNAGSGS |
| | | GFNGGFGSSMDSKSSGWGM |
| 6 | SynIDR | SKGPGRGDSPY |
| | | SGRGDSPYSGRGDSPYSGRG |
| | | DSPYSGRGDSPYSGRGDSPY |
| | | SGRGDSPYSGRGDSPYSGRG |
| | | DSPYSGRGDSPYSGY |

### Plasmids and Lentivirus

DNA fragments encoding CAR or antigen molecules was inserted into a pHR lentiviral vector with an SFFV promoter and a WPRE terminator. HEK293T cells, maintained in DMEM medium supplemented with 10% FBS and a Glutamine-Penicillin-Streptomycin mix, were co-transfected with the pHR plasmids and second-generation lentiviral packaging plasmids pMD2. G and psPAX2 (Addgene plasmid #12259 and #12260) using Genejuice transfection reagent (EMD Millipore, #70967-3). 48 hrs after transfection, cell culture media containing viral particles were harvested, centrifuged, and filtered through 0.45 μm pore size filters.

### Generation of CAR-T Cells

Pan T cells were isolated from PBMCs (Zenbio #SER-PBMC-200) from healthy donors using EasySep™ Human T Cell Isolation Kit (Stem Cell Cat #17951). T cell proliferation was stimulated with Human T-Activator CD3/CD28 Dynabeads (ThermoFisher, #11161D). Cells were cultured in RPMI1640 supplemented with 10% FBS, 50 nM 2-mercaptoethanol, 300 U/mL Interleukin-2 (PEPROTECH, #200-02). Two days after stimulation, the cells were infected with fresh lentivirus via spinoculation at 800×g for 90 min at 32° C. Half of the cell culture media were changed with fresh T cell culture medium 24 hours after infection. Five days after infection, Dynabeads were removed, and CAR-T cells were resuspended in a fresh culture medium. The medium was exchanged every two days after.

### Generation of Cancer Cell Lines

The Nalm6, Raji B, and K562 cells were cultured in RPMI1640 supplemented with 10% FBS and a Glutamine-Penicillin-Streptomycin mix. HT29 cells were cultured in DMEM supplemented with 10% FBS and a Glutamine-Penicillin-Streptomycin mix. The Nalm6 CD19-high and -low cell lines expressing GFP and Luciferase were used. The Raji B CD19-high and -low cells were generated by sorting the wild-type Raji B cells for high and low CD19 expression level. The K562 HER2-high and -low cells were generated by infecting K562 cells with lentivirus encoding the wild-type HER2, followed by single-cell sorting. The number of antigen molecules per cell was quantified using the BD Quantibrite PE Phycoerythrin Fluorescence Quantitation Kit (BD Bioscience, Cat #340495) by flow cytometry. Luciferase-expressing cells were generated by infecting cancer cells with a lentiviral plasmid expressing Luciferase-GFP or Luciferase-mCherry, followed by FACS to generate stable cell lines.

### In Vitro Cytotoxicity Assay

Cytotoxicity was measured by luciferase assay. Cancer cells expressing luciferase were resuspended in RPMI medium supplemented with 10% FBS and mixed with CAR-T cells at an effector to target ratio from 0.3:1 to 10:1. After 24 hours of incubation at 37° C., cells were collected and washed with PBS. Cell pellets were lysed in a lysis reagent (Promega, Cat #1531). The luminescence of lysates was detected by Luciferase Assay System (Promega, Cat #E1500) and analyzed using a plate spectrophotometer. The spontaneous release control was set up using cancer cells alone. Cell lysis %=[1−(experimental readout−spontaneous readout)/spontaneous readout]×100.

### Cytokine Production

CAR-T cells and cancer cells were cocultured at indicated E:T ratios for 24 hours at 37° C. The supernatant was collected for cytokine measurement using ELISA kits (IL2 ELISA kit, BioLegend #431801; IFNγ ELISA kit, BioLegend #430101; TNFα ELISA kit, BioLegend #430204) or using LEGENDplex™ Human CD8/NK Panel Kit (Cat #741186), according to the manufacturer's instruction.

### Flow Cytometry

To determine the cell-surface expression, cells were collected and blocked with an anti-human Fc receptor binding inhibitory antibody in the staining buffer (PBS with 2% FBS and 1 mM EDTA) for 15 minutes at 4° C., which were further incubated with individual antibodies in the staining buffer for 30 minutes on ice. The stained cells were washed twice with the staining buffer before sending for flow cytometry analysis. To determine the intracellular expression of targets of interest, cells were collected and fixed with Fixation/Permeabilization Solution (Cat #554714) for 15 min on ice, blocked in the staining buffer for 30 min on ice, followed by antibody staining. To characterize T cells in the mice blood, blood was drawn from a tail cut and diluted into PBS supplemented with 3 mM EDTA. Red blood cells were lysed with a red blood cell lysis buffer. The rest of cells were stained as described above and followed by flow cytometry. To characterize tumor-infiltrating T cells, tumors were dissected and digested with RPMI medium containing 0.5 mg/mL Collagenase P and 1 μg/mL DNase per 100 mg of tumor tissues for 30 min at 37° C. on a shaker. The digested tumor tissues were further homogenized and passed through a 40 μm strainer, followed by centrifugation to collect cell samples. Cells were further stained and analyzed by flow cytometry as described above.

### Mouse Tumor Xenograft

Mice were housed in pathogen-free conditions and cared for in accordance with US National Institutes of Health guidelines, and all procedures were approved by the Yale University Animal Care and Use Committee. To generate liquid cancer models, 1 million of Raji B or Naml6 cells in 100 μL of PBS were intravenously injected into the NSG (NOD.Cg-Prkdcscid Il2rgtm1Wjl/SzJ) mice (~6 weeks old) via tail vein. Three days later, 8 million of the control or IDR CAR T cells in 100 uL PBS were injected by tail vein. In vivo imaging of bioluminescence was performed to monitor tumor growth. Blood was drawn at indicated time points post CAR-T treatment for flow cytometry analysis of CAR-T. To generate solid tumor models, 2.5 million HT29 cells in 100 uL PBS were subcutaneously injected into the right flank of NSG mice. Eight days later, when tumor became palpable, 8 million of CAR T cells in 100 uL PBS were injected by tail vein, which was followed by a second dose treatment of 4 million of CAR T cells after five days. Tumor growth was measured with calipers every two days, and the size was calculated as one-half of the product of perpendicular length and square width in cubic millimeters (Volume=½*L*W*W). Mice were euthanized when the tumor size exceeded 2,000 mm³. Blood was collected and tumors were dissected to quantify tumor-infiltrating T cells.

### Signaling Characterization in CAR-T

CAR-T cells were resuspend in image medium (RPMI1640 no phenol red supplemented with 20 mM HEPES pH 7.4) and co-cultured with cancer cells at E:T=1:1 for indicated time at 37° C., followed by fixation and permeabilization on ice for 15 min, and staining with individual phosphor-antibodies before sending for flow cytometry analysis or confocal microscopy.

### Microscopy

TIRF and Confocal microscopy was performed on a Nikon Ti2-E inverted motorized microscope stand equipped with motorized stage with stagetop Piezo, Nikon H-TIRF, Yokogawa CSU-X1 spinning disk confocal, Agilent laser combiner with four lines, 405, 488, 561, and 640 nm, and scientific CMOS camera Photometrics Prime 95B. Images were acquired using Nikon Elements. To monitor CAR localization on the cell surface, glass was coated with 10 nM ICAM-1 (Sinobiological #10346-H08H) in PBS for 2 hrs at room temperature. CAR-T cells resuspended in imaging medium (RPMI1640 no phenol red supplemented with 20 mM HEPES pH7.4) were dropped onto the glass and examined by TIRF microscopy. To examine cell-cell conjugates and synapse, CAR-T cells and cancer cells were fixed in imaging media at E:T=1:I for 30 min at 37° C. The cell mixture was dropped onto glass and imaged by confocal microscopy. To measure signaling in the CAR-T synapse, CAR-T cells were co-cultured with cancer cells for 5 min at 37° C. before fixation and permeabilization for 15 min on ice, followed by staining with phosphor-antibodies for 30 min on ice. The stained cells were dropped onto glass and imaged by confocal microscopy.

### Image Analysis

Microscopy images were analyzed in Fiji (ImageJ). The same brightness and contrast were applied to images within the same panels. CAR condensation was quantified as normalized variance, which equals to the square of standard deviation divided by the mean. Cell conjugation percentage was calculated by dividing the number of tumor cell-associated GFP+CAR-T cells by the number of total GFP+CAR T cells. CAR activation was calculated by dividing the intensities of pCD3ζ (Y142) or pLAT (Y171) in the synapse by the intensities of CAR-GFP.

### Statistical Analysis

Student's T test or Mann-Whitney U test analysis was used to assess significance, with P<0.05 considered significant. Data were analyzed on GraphPad Prism. The statistical details for each experiment were provided in the associated figure legends.

### Example 1: Construction and Validation of Chimeric Antigen Receptors Fused with Intrinsically Disordered Regions (CAR-IDR)

IDRs contain diverse sequence and structure features. To determine which IDR promotes the condensation of CAR, candidates were selected from 6 well-characterized IDRs that were previously shown to induce condensation in a cellular environment. These include IDRs from FUS, EWS, TAF15, Nup98, TDP43, and a synthetic IDR (synIDR) (Chong et al., 2018; Dzuricky et al., 2020; Kato et al., 2012; Protter et al., 2018; Wang et al., 2018; Xu et al., 2021; You et al., 2020). CD19 CAR was selected, which is commonly used in research and clinical practice, as an initial model. This CAR is composed of a single chain variable fragment (scFv, FMC63) that targets CD19, a stalk and transmembrane domain from CD8a, and cytosolic signaling domains from 41BB, CD28, and CD3ζ. The IDR was fused to the C-terminus of CD3ζ. A superfolder GFP tag, which promotes the folding of fused client proteins (Pedelacq et al., 2006), was further attached on the C-terminus of IDR for visualization of CAR condensation (FIG. 1A). The superfolder GFP tag facilitates live cell imaging, which avoids potential fixation-induced artifacts in characterizing IDR condensation (Irgen-Gioro et al., 2022). The DNA fragment encoding the control or IDR CAR was packaged into lentivirus and introduced into primary CD3+ T cells purified from the human peripheral mononuclear cells (PBMCs). Flow cytometry revealed the total cellular expression (by GFP) versus cell surface localization (by FMC63) of individual CARs (FIG. 1B-1I), demonstrating that fusion with IDR did not affect the trafficking of CAR to the cell surface. To visualize the condensation of CAR on the cell surface, live CAR-T cells were stained with a plasma membrane dye CellMask deep red and performed total internal reflection fluorescence (TIRF) microscopy which effectively reduces the cytosolic background of fluorescence. It was found that CAR fused with FUS, EWS, or TAF15 displayed an enhanced condensation as compared to the control CAR (FIG. 1J). Therefore, these three CARs in the following functional assays were focused.

The aggregation of CARs targeting GD2 or CSPG4 induces tonic signaling, which triggers CAR-T activation in the absence of antigens (Chen et al., 2023; Long et al., 2015). Therefore, if IDR-induced CAR condensation causes tonic signaling was assessed. It was found that the IDR CAR-Ts did not proliferate in the absence IL-2 (FIG. 1K). The expression of CD69, a T cell activation marker, was similar between the control and IDR CAR-Ts in the absence of antigen (FIG. 7A). Moreover, IDR CAR-Ts did not secret detectable TNFα in the absence of antigen (FIG. 7B). Together, assessed from cell proliferation, activation marker, and cytokine production, IDR-induced CAR condensation did not trigger tonic signaling.

### Example 2: IDR-Fusion Enhances the Cytotoxicity of CD19 CAR

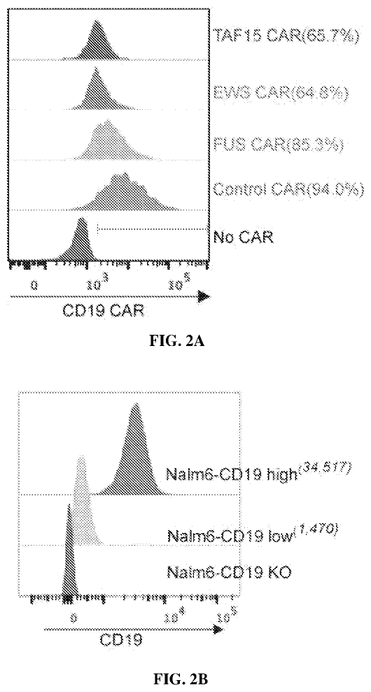

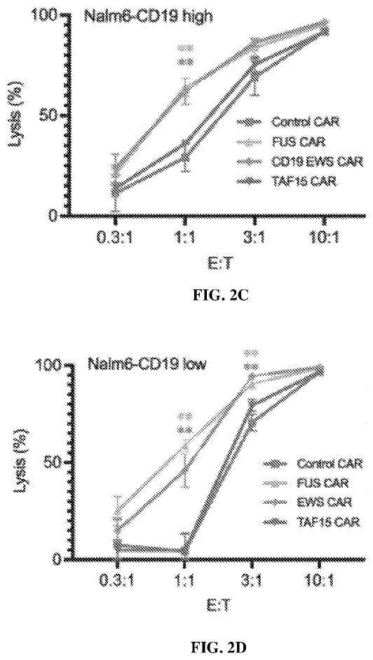

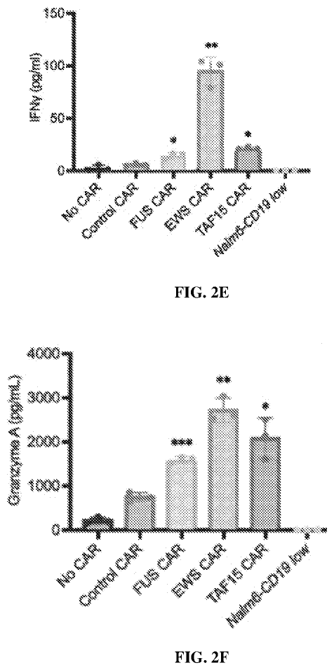

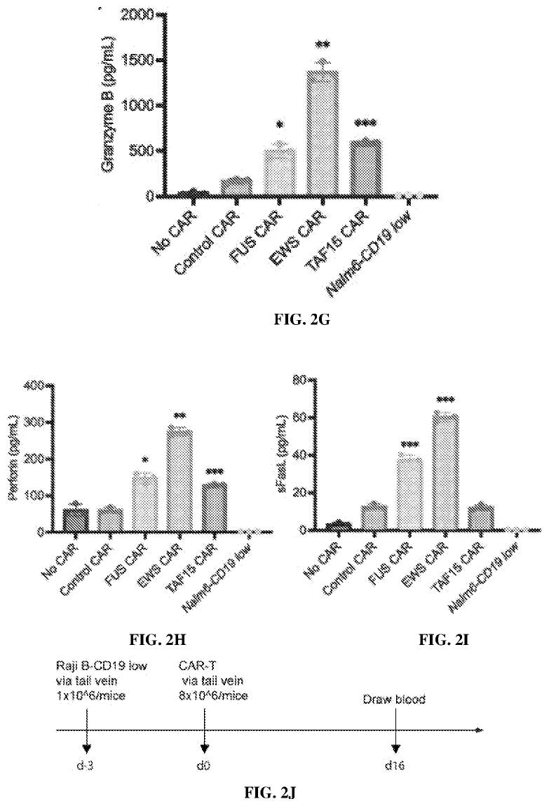

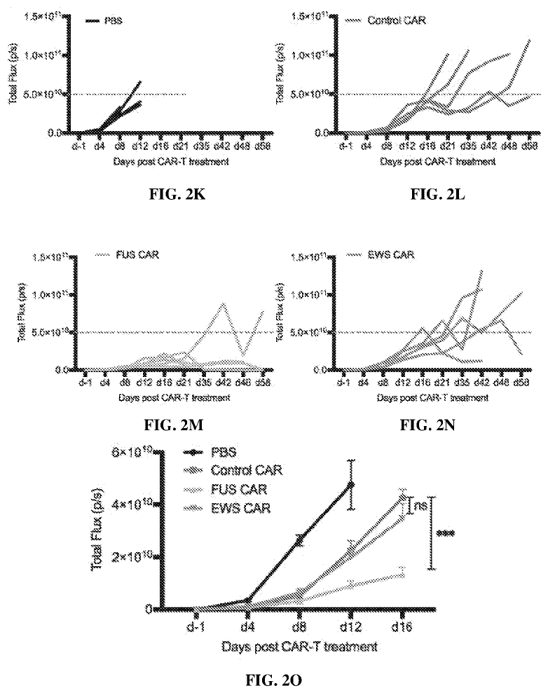

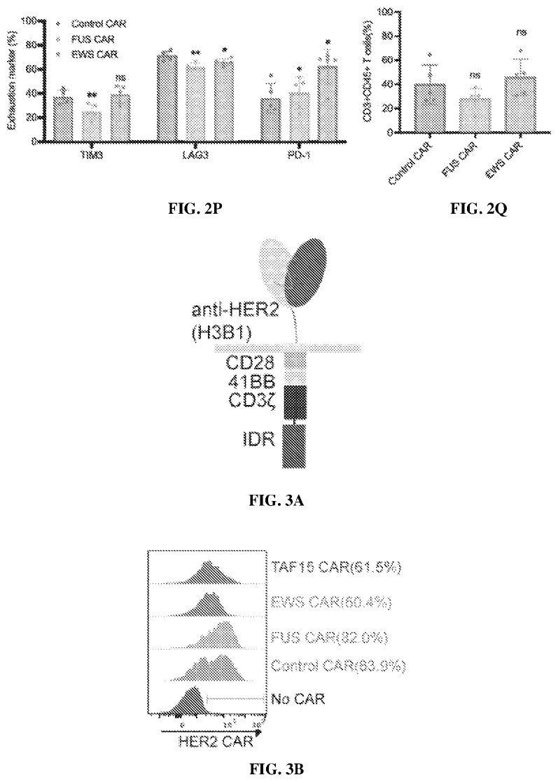

To determine how IDRs affect the cytotoxicity of CAR-Ts against cancer cells, the control or IDR CAR-Ts (FIG. 2A) were co-cultured with variants of Nalm6, a B cell leukemia line, that expresses either high or low CD19 (FIG. 2B). The Nalm6 cells express a luciferase reporter which facilitates the quantification of cytotoxicity by the luciferase assay. The FUS and EWS, but not TAF15 CAR, displayed a substantial higher cytotoxicity towards both CD19-high and low Nalm6 cells (FIGS. 2C-2D).

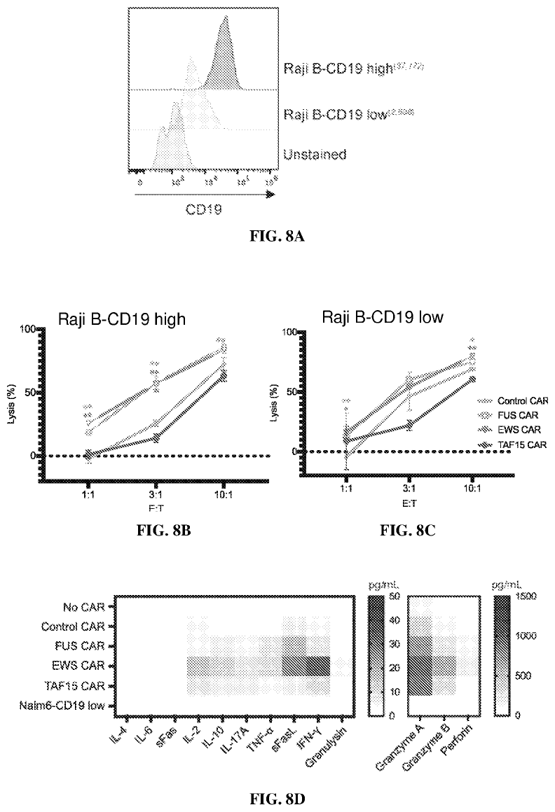

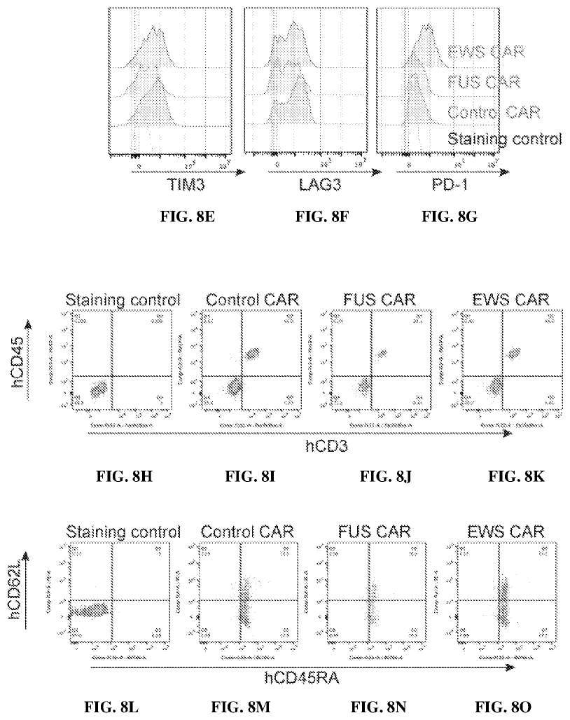

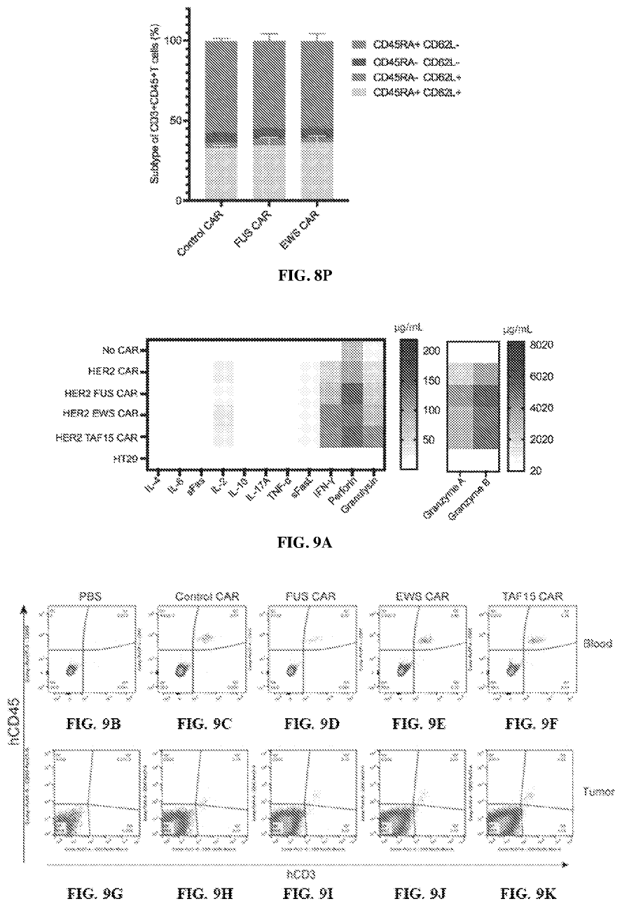

This result was recapitulated using another B cell line Raji as the target (FIGS. 8A-8C). The higher cytotoxicity of FUS and EWS CAR could be explained by their higher secretion of cytotoxic factors including Granzyme A, Granzyme B, Perforin, FASL, and IFNγ, when CAR-Ts were engaged with CD19-low Nalm6 cells (FIGS. 2E-2I and FIG. 8D). Together, these data showed that the FUS and EWS IDRs enhanced the cytotoxicity of CAR-Ts against CD19-low cells, which was accompanied with a higher secretion of cytotoxic factors.

To assess the tumor killing effect of IDR CAR-Ts toward CD19-low cancer cells in vivo, CD19-low Raji B cells expressing a luciferase reporter were injected into the immune-deficient NSG mice intravenously. Three days later, the control, FUS, or EWS CAR-T cells were infused through the tail vein. The cancer progression was monitored by bioluminescence imaging (FIG. 2J). It was determined that FUS CAR-T inhibited cancer proliferation better than the control CAR-T (FIGS. 2K-20). The expression of T cell exhaustion marker TIM3 and LAG3 were lower in FUS CAR-T (FIG. 2P and FIG. 8E-8G). No significant difference was detected in the T cell number (FIG. 2Q and FIG. 8H-8K) or memory phenotype (FIG. 8L-8P). Together, these data indicated that FUS CAR-T exhibited an enhanced anti-cancer activity both in vitro and in vivo.

### Example 3: IDRs Enhanced Cytotoxicity of HER2 CAR

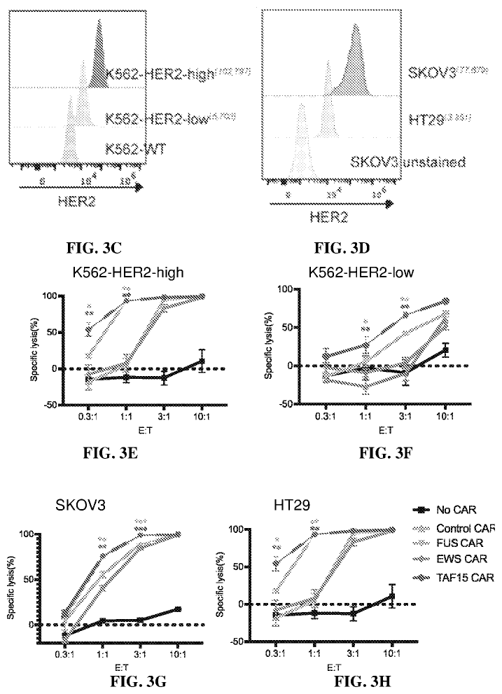

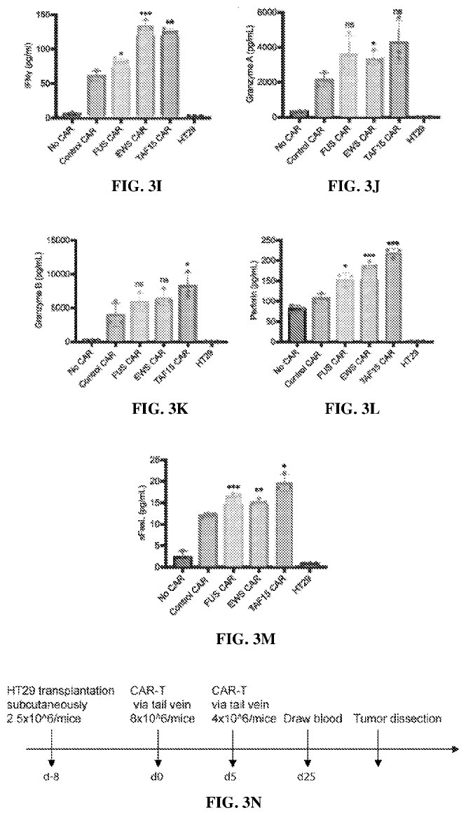

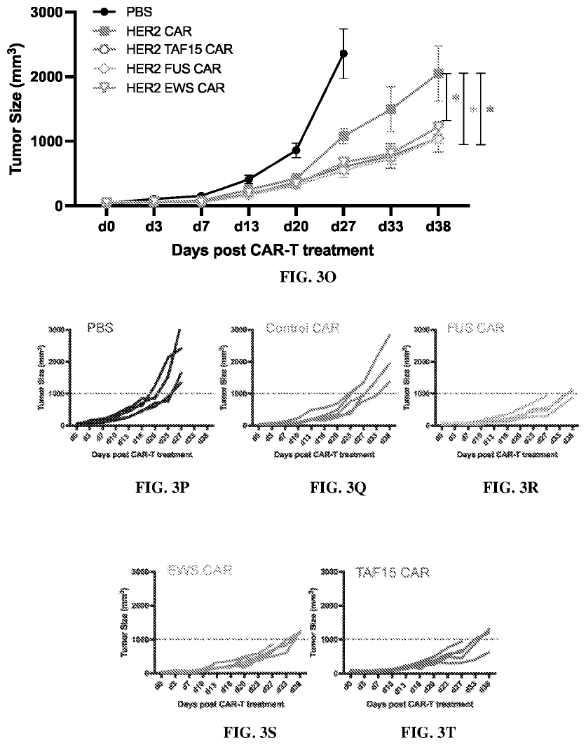

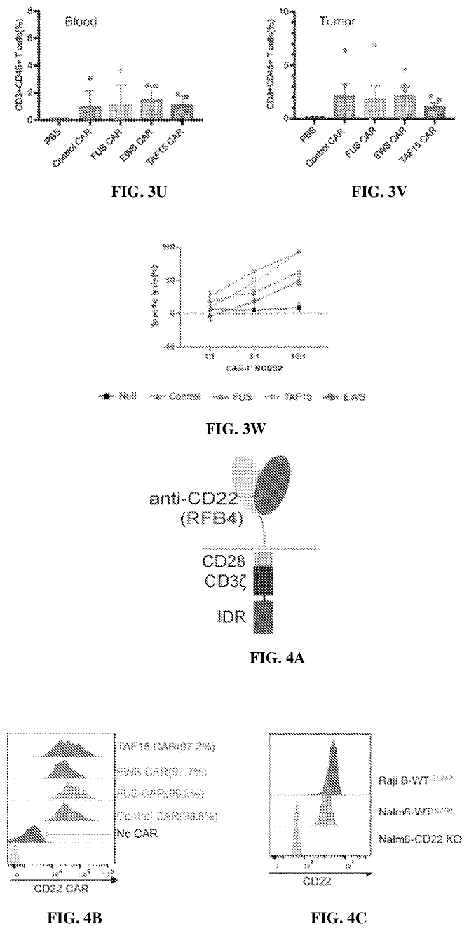

To determine if the effect of IDR in promoting cytotoxicity applied to CARs beyond CD19, IDR CARs targeting HER2 (FIG. 3A), an antigen commonly overexpressed in multiple solid tumors including breast, ovarian, lung and colorectal cancers, were constructed. Human primary T cells were infected with lentivirus encoding the control, FUS, TAF15, or EWS CAR (FIG. 3B). Similar to the case of CD19 CAR, IDR did not trigger tonic signaling of HER2 CAR as assessed by CD69 expression and TNFα expression (FIGS. 7C-7D). Next, multiple target cell lines were selected for testing cytotoxicity: the lymphoblast K562 cells ectopically expressing high or low HER2 (FIG. 3C), an ovarian cancer cell line SKOV3 expressing high HER2 and a colon cancer cell line HT29 expressing low HER2 (FIG. 3D). These target cells were co-cultured with the control or IDR CAR-Ts. It was found that FUS and TAF15 CAR-Ts displayed a higher cytotoxicity towards all four cell lines tested, as compared to the control CAR-T (FIGS. 3E-3H).

Consistent with that, FUS and TAF15 CAR-Ts secreted a higher level of cytotoxic factors including IFNγ, Perforin, and FasL, than the control CAR-T (FIGS. 3I-3M and FIG. 9A). Together, these data indicated that FUS and TAF15 enhanced the cytotoxicity of HER2-high and low CAR-Ts in vitro.

To determine the anti-tumor effect of IDR CAR-Ts toward HER2-low cells in vivo, HT-29 cells were injected into the immune-deficient NSG mice subcutaneously. Eight days later, the control or IDR CAR-T cells were infused through the tail vein. The tumor progression was monitored by electronic caliper over 7 weeks (FIG. 3N). It was determined that FUS and TAF15 CAR-T inhibited tumor growth better than the control CAR-T (FIGS. 3O-3T), which is consistent with the in vitro killing results. Although EWS CAR-T did not show a higher cytotoxicity in vitro, it did display a higher anti-tumor effect than the control CAR-T. This could be potentially explained by the higher cytotoxic factors, including IFNγ, that were secreted by EWS CAR-T (FIG. 3I). The numbers of circulating and tumor-infiltrating T cells were comparable between the control and IDR CAR-Ts (FIGS. 3U-3V and FIGS. 9B-9K), indicating that the enhanced killing was not related to an increased cell number but to an increased cytotoxicity (FIG. 3H). Together, these data indicated that IDR CAR-Ts exhibited an enhanced anti-cancer efficacy towards HER-low cells in vivo.

### Example 4: IDRs Enhanced Anti-Tumor Effect of CD22 CAR In Vivo

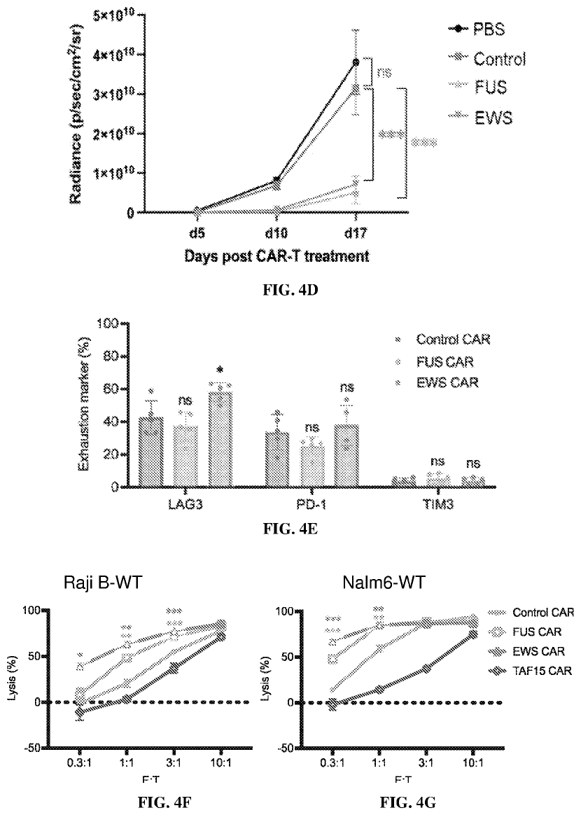

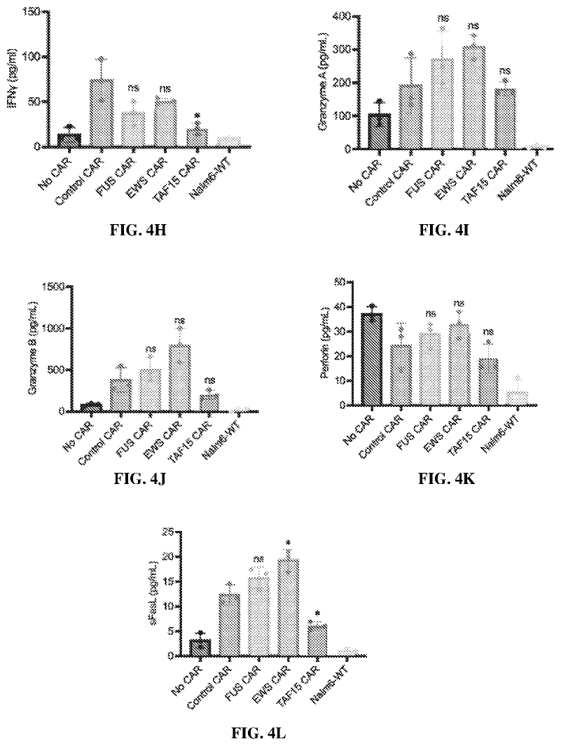

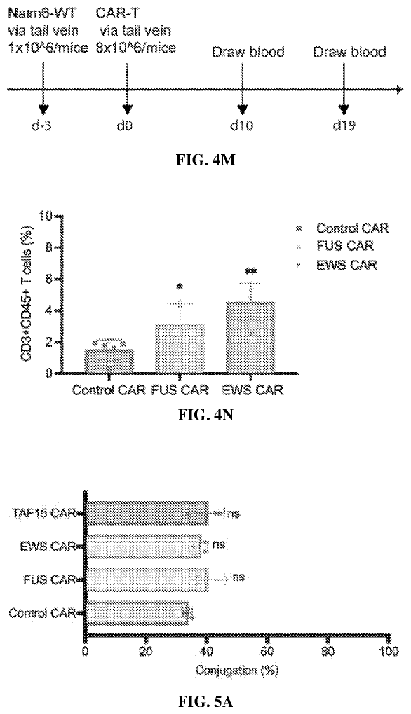

In addition to the anti-CD19 and anti-HER2 CAR which target membrane-proximal epitopes, how IDRs affect the cytotoxicity of an anti-CD22 CAR (RFB4) were tested, which showed very low signaling efficiency due to the membrane-distal epitope position on CD22 (James et al., 2008; Xiao et al., 2022b). Using a similar design strategy to the CD19 CAR, the IDR CARs targeting CD22 were constructed by fusing the IDR on the C-terminus (FIG. 4A). Human primary T cells were infected with lentivirus encoding the control, FUS, EWS, or TAF15 CAR (FIG. 4B). The wild-type Raji B or Nalm6 cells which express a medium level of CD22 (FIG. 4C) were co-cultured with CAR-Ts.

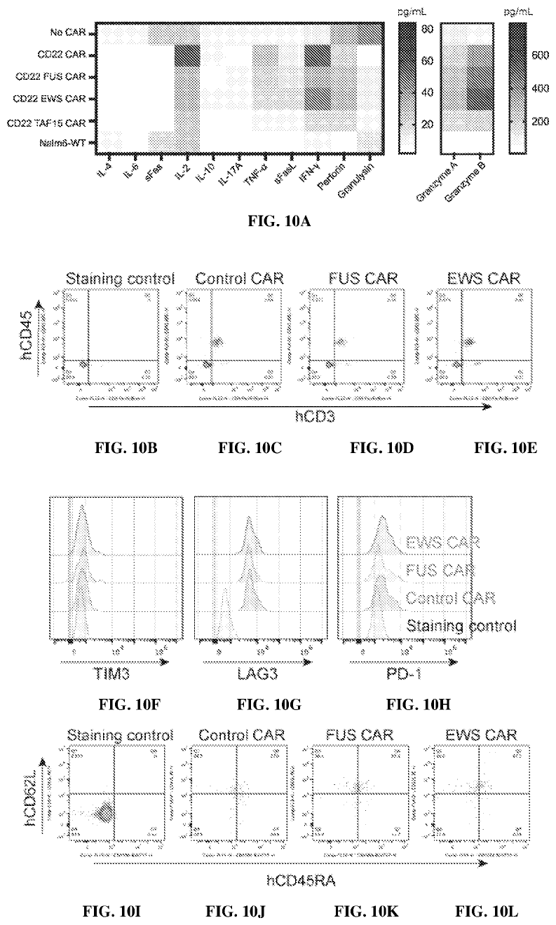

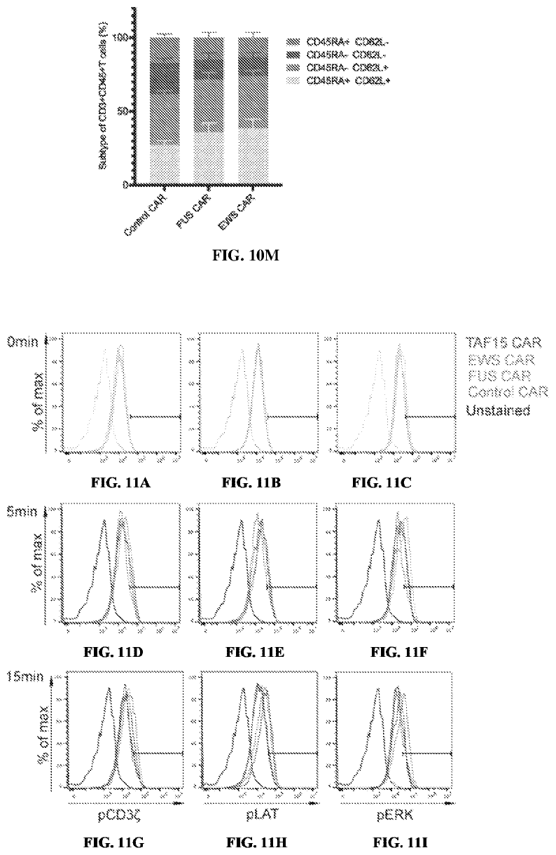

It was determined that FUS and EWS CAR-T displayed a higher cytotoxicity as compared to the control CAR-T when co-cultured with either Raji B or Nalm6 cells (FIGS. 4F-4G). The cytotoxic factors release was measured and found that FUS and EWS CAR-Ts released a slightly higher Granzyme A and B, and FasL than the control CAR-T, though it was not statistically significant (FIGS. 4H-4L and FIG. 10A). Together, these data showed that FUS and EWS enhanced the cytotoxicity of a low-signaling CD22 CAR-T.

To determine if IDRs improve the anti-tumor efficacy of the above low-signaling CD22 CAR in vivo, the wild-type Nalm6 cells were injected into the NSG mice intravenously. Three days later, the control, FUS or EWS CAR-T cells were infused through the tail vein. The cancer progression was monitored by bioluminescence imaging (FIG. 4M). Similar to the in vitro killing result, FUS and EWS CAR-T inhibited the tumor growth better than the control CAR-T (FIG. 4D). The circulating T cell number was higher in the FUS and EWS group than the control group (FIG. 4N and FIGS. 10B-10E). The expression of exhaustion markers (FIG. 4E and FIGS. 10F-10H) or memory phenotypes (FIGS. 10I-10M) was not significantly different between the control and IDR CAR-Ts. Together, these data indicated that IDRs from FUS and EWS promote the anti-tumor effect of a low-signaling CD22 CAR.

### Example 5: IDR Enhanced CAR-T Activation by Promoting CD3ζ and LAT Phosphorylation

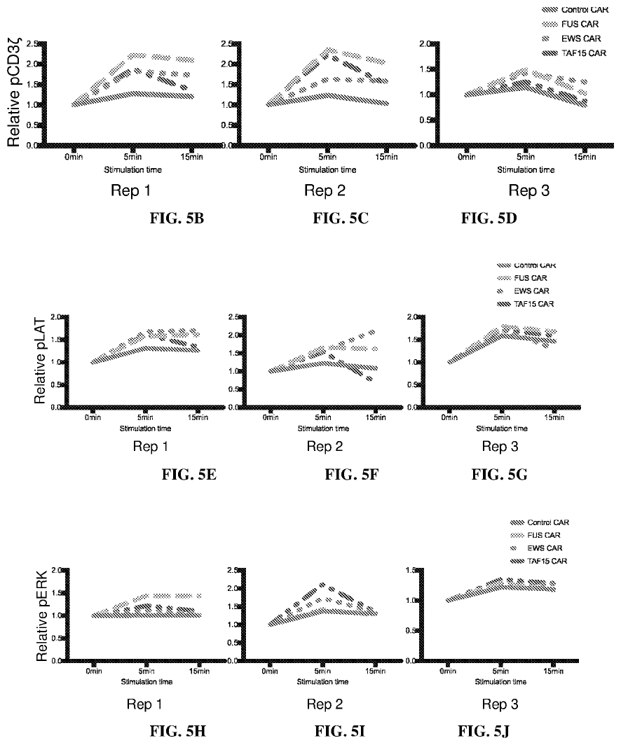

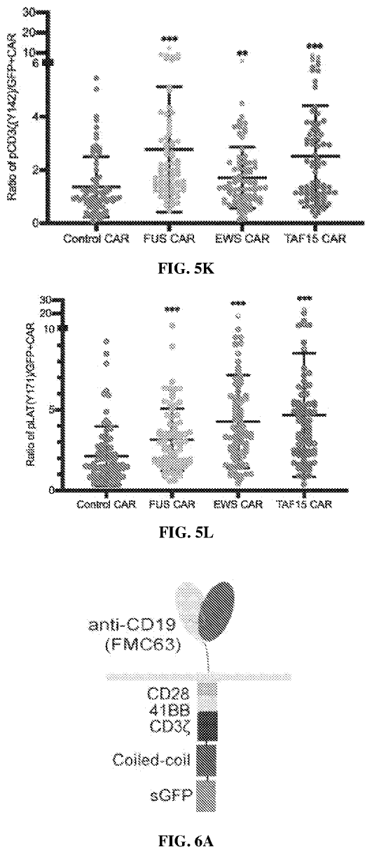

To determine the mechanism by which IDRs promote CAR-T activation, membrane-proximal signaling in CAR-Ts were monitored upon engagement with CD19. Firstly, the cell-cell conjugation efficiency between the CAR-T and cancer cells was determined as a reflection of binding strength between CAR and antigen. It was determined that IDRs did not affect the cell-cell conjugation (FIG. 5A), indicating that IDRs promote CAR-T activation post CAR-antigen interactions. Following a CAR-antigen engagement, the CD3ζ domain on CAR is phosphorylated, which leads a cascade of biochemical reactions occurring at the T cell synapse and intracellular space. CAR-Ts were co-cultured with CD19-low Raji B cells and the antigen-dependent signaling kinetics by flow cytometry was monitored. It was determined that the FUS, EWS, and TAF15 IDR enhanced the phosphorylation of CD3ζ, LAT, ERK, which lined up a pathway that mediates IDR CAR-T activation (FIGS. 5B-5J and FIGS. 11A-11I). Because CD3ζ and LAT are two transmembrane proteins enriched in the CAR-T synapse, their phosphorylation level in the synapse by confocal microscopy was examined. Consistent with the flow cytometry measurement, all three IDRs enhanced the phosphorylation of CD3ζ and LAT in the synapse (FIGS. 5K-5L). Together, these data indicated that IDRs promote CAR-T activation against low-antigen cancer cells by promoting membrane-proximal signaling pathways.

### Example 6: Oligomerization of CAR by Coiled-Coil Domains Caused Reduced Surface Localization

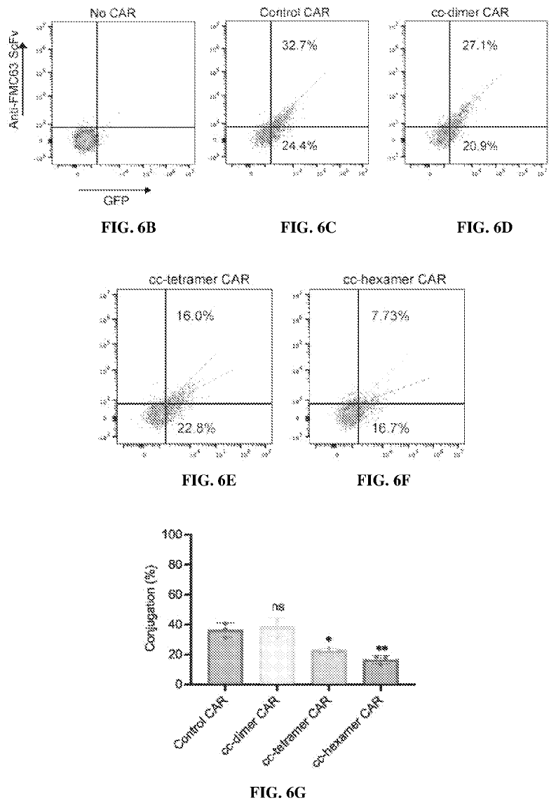

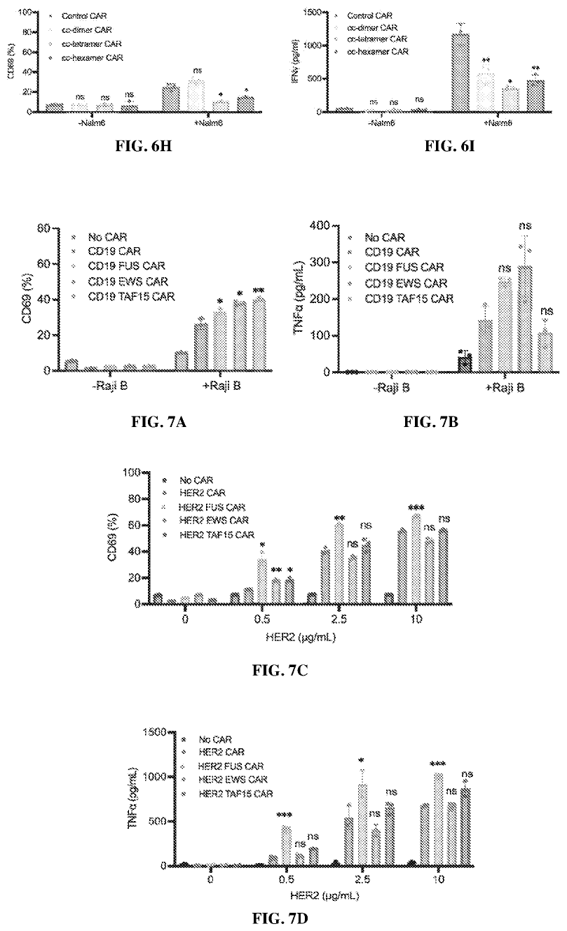

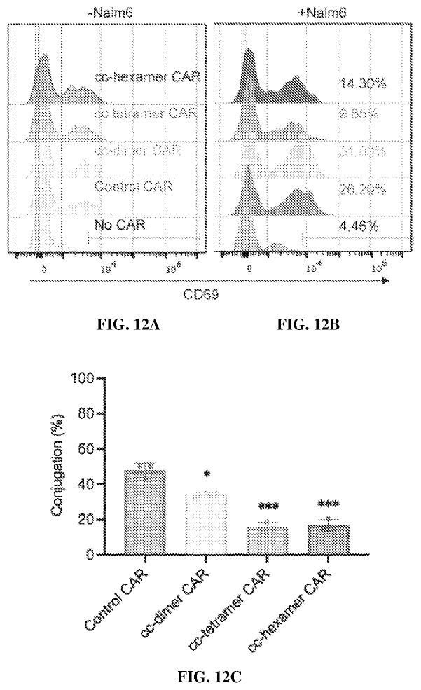

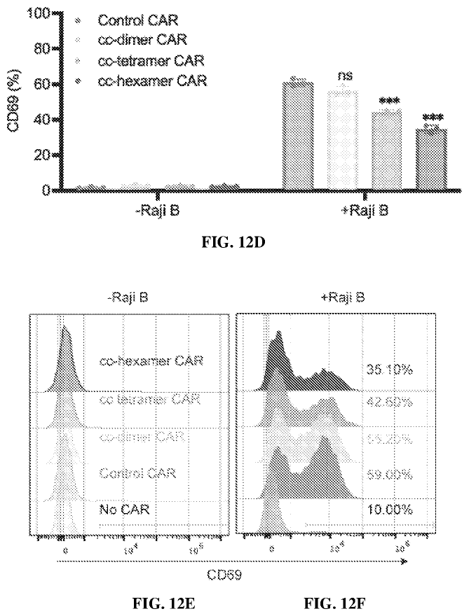

In addition to IDR-mediated protein condensation, the coiled-coil domain is a commonly used tool to induce oligomerization of protein of interest. To test if coiled-coil domains could promote CAR-T activation, coiled-coil (cc) domains was fused that mediate dimerization, tetramerization, or hexamerization (Deng et al., 2006; Spencer and Hochbaum, 2017) to the C-terminus of a CD19 CAR (FIG. 6A) and introduced these cc-CARs into human primary T cells. Whereas the cc-dimer maintained a similar cell surface expression level as compared to the control CAR, cc-tetramer and hexamer CAR showed a dramatic reduction in the cell surface localization (FIG. 6B-6F). Consequently, the cell-cell conjugation percentage between CAR-T and Nalm6 cells was significantly reduced in the cc-tetramer and hexamer as compared to the control CAR (FIG. 6G). In consistency, CAR-T activation, as evaluated by CD69 expression (FIG. 6H and FIGS. 12A-12B) and IFNγ secretion (FIG. 6I), was significantly reduced in cc-tetramer or hexamer. The reduction in cell-cell conjugation and CAR-T activation were recapitulated when using Raji B as a target cell (FIGS. 12C-12F). Similar to CAR, the oligomerization-induced receptor internalization was frequently observed in other transmembrane receptors including EGFR and FGFR (Hofman et al., 2010; Pozniak et al., 2020). The fact that IDRs do not affect cell surface expression of CAR indicated that IDRs present a unique advantage to promote CAR clustering without causing enhanced receptor internalization.

### SUMMARY

Chimeric antigen receptor (CAR)-T cell-based therapies demonstrate remarkable efficacy for the treatment of otherwise intractable cancers, particularly B-cell malignancies. However, existing FDA-approved CAR-Ts are limited by low antigen sensitivity, rendering their insufficient targeting to low antigen-expressing cancers. To improve the antigen sensitivity of CAR-Ts, CARs targeting CD19, CD22, and HER2 were engineered by including intrinsically disordered regions (IDRs) that promote signaling condensation. The “IDR CARs” triggered enhanced membrane-proximal signaling in the CAR-T synapse, which led to an increased release of cytotoxic factors, a higher killing activity towards low antigen-expressing cancer cells in vitro, and an improved anti-tumor efficacy in vivo. No elevated tonic signaling was observed in IDR CAR-Ts. Together, IDRs are a new tool set to enhance CAR-T cytotoxicity and to broaden CAR-T's application to low antigen-expressing cancers.

Biomolecular condensation has been demonstrated to regulate diverse cellular signaling processes (Case et al., 2019; Li et al., 2012; Li et al., 2022). Previous work showed that condensation of TCR signaling molecules promotes membrane-proximal signaling including LAT phosphorylation, RAS activation, actin polymerization, and ERK activation (Huang et al., 2019; Su et al., 2016; Zeng et al., 2021). The data and results presented herein identified IDR-induced condensation as a new strategy to improve the function of CAR-T. In this study, it was demonstrated the proof-of-concept application of implementing IDRs to enhance the cytotoxicity of CAR-Ts without inducing tonic signaling. IDRs are present in 30% of the human proteins, and it is believed that many more IDRs can modulate the condensation, antigen-binding, and conformation of CARs.

In this work, it was demonstrated that several well-characterized IDRs including those from FUS, EWS, and TAF15, promoted the condensation of CARs on the T cell membranes, which leads to an enhanced cytotoxicity towards low antigen-expressing cancer cells. Because the three above IDR-containing proteins are naturally functioning in the nucleus and none of these IDRs have been reported to have a function in T cell signaling or cytotoxicity, current strategy can serve as an orthogonal means to enhance the low signaling efficiency of CAR-T, in combination with other strategies, including selecting specific transmembrane or co-signaling domains (Heitzeneder et al., 2022; Majzner et al., 2020; Priceman et al., 2018), adding new signaling binding motifs (Salter et al., 2021), replacing the intracellular part with that from TCR (HIT) (Mansilla-Soto et al., 2022), dual targeting by CAR together with chimeric costimulatory receptors (Katsarou et al., 2021), or implementing other signaling pathways (Wilkens et al., 2022), to achieve extra combined effects.

Not all the IDRs tested in the current study promoted CAR condensation according to the tests utilized. FUS, EWS, or TAF15 promoted CAR condensation, but not NUP98, TDP43 or SynIDR according to the initial screening test. This could be attributed to the fact that the ability of these IDRs to promote condensation was initially characterized in the 3D cytosolic environment, which is different from the 2D plasma membrane environment where CAR is localized. The local molecular crowdedness or the negative charge in the inner leaflet of plasma membrane could influence the ability of individual IDRs to promote CAR condensation.

Current work showed that IDRs promoted LAT phosphorylation in the CAR synapse. LAT is one of the key adaptor proteins in the TCR pathway; it forms signaling condensates, recruits and activates a plethora downstream effector to amply TCR signaling (Bunnell et al., 2002; Zhang et al., 1998). Previous studies indicated that LAT is poorly activated (phosphorylated) following CAR-antigen interactions (Dong et al., 2020; Salter et al., 2021), which could explain the low antigen sensitivity of CAR signaling. Here it was demonstrated that IDRs can significantly increase the activation of LAT and downstream pathways including SLP76 and ERK, which finally lead to an enhanced cytotoxicity.

A difference was also revealed between the outcomes of different ways to induce CAR clusters. IDR-induced CAR condensation enhanced CAR phosphorylation and CAR-T activation whereas coiled-coil domains reduced cell surface expression of CAR and suppressed CAR-T activation. One of the major differences between IDR and coiled-coil is that IDRs promote self-assembly of CAR through weak interactions and the condensates remain liquid-like or gel-like properties. In contrast, coiled-coil domains induced stable oligomerization which could either affect the trafficking of CAR to the cell surface or trigger instant receptor internalization. Current work demonstrated that IDRs present a distinct advantage in promoting the assembly of CAR into a higher order structure without compromising their cell surface localization.

TABLE 1
Reagents

| | Cell lines | Additional information |
|---|---|---|
| 1 | HEK293T | For lentivirus production |
| 2 | Raji B | |
| 3 | Raji B-CD19 high-ffLuc2-mCherry | For testing CD19 CAR |
| 4 | Raji B-CD19 low-ffLuc2-mCherry | For testing CD19 CAR |
| 5 | Nalm6-CD19 high-ffLuc2-GFP | For testing CD19 CAR |
| 6 | Nalm6-CD19 low-ffLuc2-GFP | For testing CD19 CAR |
| 7 | Nalm6-ffLuc2-GFP | For testing CD22 CAR |
| 8 | Nalm6-CD22 high-ffLuc2-mCherry | For testing CD22 CAR |
| 9 | Nalm6-CD22 low-ffLuc2-mCherry | For testing CD22 CAR |
| 10 | K562 | |
| 11 | K562-HER2 high-ffLuc2-mCherry | For testing CD22 CAR |
| 12 | K562-HER2 low-ffLuc2-mCherry | For testing CD22 CAR |
| 13 | SKOV3 | |
| 14 | SKOV3-ffLuc2-mCherry | For testing CD22 CAR |
| 15 | HT29 | |
| 16 | HT29-ffLuc2-mCherry | For testing CD22 CAR |

TABLE 2
Plasmids

| | | |
|---|---|---|
| 1 | pMD2.G | Viral packaging |
| 2 | poPAX | Viral packaging |
| 3 | pHR-mCherry-CAAX | Lentiviral vector |
| 4 | pFUGW-FerH-ffLuc2-mCherry | Lentiviral vector |
| 5 | pFUGW-FerH-ffLuc2-GFP | Lentiviral vector |
| 6 | pHR-CD19-mCherry | Lentiviral vector |
| 7 | pHAGE-ERBB2 (HER2) | Lentiviral vector |
| 8 | pHR NdeI-SP-CD19 ScFv-BamHI- | Lentiviral vector |
| | myc-CD8 Stalk-CD8 TM-NruI-CD28- | |
| | 41BB-CD3z cyto-speI-sGFP | |
| 9 | pHR NdeI-SP-CD19 ScFv-BamHI- | Lentiviral vector |
| | myc-CD8 Stalk-CD8 TM-NruI-CD28- | |
| | 41BB-CD3z cyto-speI-FUS 1-214- | |
| | sGFP | |
| 10 | pHR NdeI-SP-CD19 ScFv-BamHI- | Lentiviral vector |
| | myc-CD8 Stalk-CD8 TM-NruI-CD28- | |
| | 41BB-CD3z cyto-speI-TAF15 1-180- | |
| | sGFP | |
| 11 | pHR NdeI-SP-CD19 ScFv-BamHI- | Lentiviral vector |
| | myc-CD8 Stalk-CD8 TM-NruI-CD28- | |
| | 41BB-CD3z cyto-speI-EWS 28-262- | |
| | sGFP | |
| 12 | pHR NdeI-SP-CD19 ScFv-BamHI- | Lentiviral vector |
| | myc-CD8 Stalk-CD8 TM-NruI-CD28- | |
| | 41BB-CD3z cyto-speI-Nup98 1-340- | |
| | sGFP | |
| 13 | pHR NdeI-SP-CD19 ScFv-BamHI- | Lentiviral vector |
| | myc-CD8 Stalk-CD8 TM-NruI-CD28- | |
| | 41BB-CD3z cyto-TDP43 266-414 | |
| | speI-sGFP | |
| 14 | pHR NdeI-SP-CD19 ScFv-BamHI- | Lentiviral vector |
| | myc-CD8 Stalk-CD8 TM-NruI-CD28- | |
| | 41BB-CD3z cyto-10xIDR-speI-sGFP | |
| 15 | pHR NdeI-SP-CD19 ScFv-BamHI- | Lentiviral vector |
| | myc-CD8 Stalk-CD8 TM-NruI-CD28- | |
| | 41BB-CD3z cyto-speI-FUS 1-214 | |
| 16 | pHR NdeI-SP-CD19 ScFv-BamHI- | Lentiviral vector |
| | myc-CD8 Stalk-CD8 TM-NruI-CD28- | |
| | 41BB-CD3z cyto-speI-TAF15 1-180 | |
| 17 | pHR NdeI-SP-CD19 ScFv-BamHI- | Lentiviral vector |
| | myc-CD8 Stalk-CD8 TM-NruI-CD28- | |
| | 41BB-CD3z cyto-speI-EWS 28-262 | |
| 18 | pHR NdeI-SP-CD22 RFB4 ScFv- | Lentiviral vector |
| | BamHI-myc-CD8 Stalk-CD8 TM- | |
| | NruI-CD28-CD3z cyto-speI-sGFP | |
| 19 | pHR NdeI-SP-CD22 RFB4 ScFv- | Lentiviral vector |
| | BamHI-myc-CD8 Stalk-CD8 TM- | |
| | NruI-CD28-CD3z cyto-speI-FUS 1- | |
| | 214 | |
| 20 | pHR NdeI-SP-CD22 RFB4 ScFv- | Lentiviral vector |
| | BamHI-myc-CD8 Stalk-CD8 TM-NruI- | |
| | CD28-CD3z cyto-speI-EWS 28-262 | |
| 21 | pHR NdeI-SP-CD22 RFB4 ScFv- | Lentiviral vector |
| | BamHI-myc-CD8 Stalk-CD8 TM- | |
| | NruI-CD28-CD3z cyto-speI-TAF15 | |
| | 1-180 | |
| 22 | pHR NdeI-SP-HER2 ScFv-BamHI- | Lentiviral vector |
| | myc-CD8 Stalk-CD28 TM-NruI- | |
| | CD28-41BB-CD3z cyto-sGFP | |
| 23 | pHR NdeI-SP-HER2 ScFv-BamHI- | Lentiviral vector |
| | myc-CD8 Stalk-CD28 TM-NruI- | |
| | CD28-41BB-CD3zcyto-speI-TAF15 | |
| | 1-180 | |
| 24 | pHR NdeI-SP-HER2 ScFv-BamHI- | Lentiviral vector |
| | myc-CD8 Stalk-CD28 TM-NruI- | |
| | CD28-41BB-CD3z cyto-speI-FUS 1-214 | |
| 25 | pHR NdeI-SP-HER2 ScFv-BamHI-myc- | Lentiviral vector |
| | CD8 Stalk-CD28 TM-NruI-41BB-CD3z | |
| | cyto-speI-EWS 28-262 | |

TABLE 3
Antibodies

| | | | |
|---|---|---|---|
| 1 | PE anti-human CD19 Antibody | BioLegend | #392506 |
| 2 | PE anti-human CD22 Antibody | BioLegend | #363503 |
| 3 | PE anti-human CD340 (erbB2/ | BioLegend | #324405 |
| | HER-2) Antibody | | |
| 4 | APC anti-human CD19 Antibody | BioLegend | #392504 |
| 5 | APC anti-human CD22 Antibody | BioLegend | #363506 |
| 6 | Alexa Fluor ® 647 anti-human | BioLegend | #324412 |
| | CD340 (crbB2/HER-2) Antibody | | |
| 7 | APC anti-human CD69 Antibody | BioLegend | #310910 |
| | (FN50) | | |
| 8 | Brilliant Violet 421 ™ anti-human | BioLegend | #310930 |
| | CD69 Antibody (FN50) | | |
| 9 | APC anti-human CD366 (TIM3) | Invitrogen | #17-3109-42 |
| | Monoclonal Antibody (F38-2E2) | | |
| 10 | PE anti-human CD279 (PD-1) | Invitrogen | #12-9969-42 |
| | Monoclonal Antibody (MIH4) | | |
| 11 | PE-Cyanine7 anti-human CD223 | Invitrogen | #25-2239-42 |
| | (LAG-3) Monoclonal Antibody | | |
| | (3DS223H) | | |
| 12 | Pacific Blue ™ Mouse Anti-Human | BioLegend | #300417 |
| | CD3 Clone UCHT1 | | |
| 13 | PerCP/Cyanine5.5 anti-human | BioLegend | #304122 |
| | CD45RA Antibody | | |
| 14 | Pacific Blue ™ anti-human CD4 | BioLegend | #317424 |
| | Antibody | | |
| 15 | BD Horizon ™ BUV496 Mouse | BDbiosciences | #612936 |
| | Anti-Human CD4 | | |
| 16 | Brilliant Violet 785 ™ anti- | BioLegend | #344739 |
| | human CD8 Antibody | | |
| 17 | PE/Cy7 anti-human CD8a | BioLegend | #301012 |
| | [RPA-T8] 100 tests | | |
| 18 | APC/Fire ™ 750 anti-human | BioLegend | #304845 |
| | CD62L Antibody | | |
| 19 | Brilliant Violet 750TM anti- | BioLegend | #304166 |
| | human CD45RA Antibody, | | |
| | 100 tests | | |
| 20 | BD PhosflowTM Alexa Fluor ® | BDbiosciences | #558489 |
| | 647 Mouse anti-CD247 (pY142) | | |
| 21 | Alexa Fluor ® 647 anti-LAT | BioLegend | #946603 |
| | Phospho (Tyr171) Antibody | | |

TABLE 4
Others

| | | | |
|---|---|---|---|
| 1 | EasySep ™ Human T Cell Isolation | Stem Cell | #17951 |
| | Kit | | |
| 2 | Human T-Activator CD3/CD28 | ThermoFisher | #11161D |
| | Dynabeads | | |
| 3 | Recomninant Interleukin-2 | PEPROTECH | # 200-02 |
| 4 | Human Recombinant Biotinylated | Sino Biological | #10004- |
| | Her2/ERBB2 protein | | H08H-B |
| 5 | Human Recombinant Biotinylated | Sino Biological | #11958- |
| | CD22 protein | | H41H-B |
| 6 | Luciferase Assay System | Promega | # E1500 |
| 7 | Cell lysis reagent | Promega | # 1531 |
| 8 | Red Blood cell lysis buffer | BioLegend | #420301 |
| 9 | D-Luciferin, Sodium Salt | Gold | #LUCNA- |
| | | Biotechnology | 100 |
| 10 | Fixation/Permeabilization Solution | BD Bioscience | # 554714 |
| 11 | TNFα ELISA kit | BioLegend | # 430204 |
| 12 | BD Quantibrite PE Phycoerythrin | BD Bioscience | #340495 |
| | Fluorescence Quantitation Kit | | |
| 13 | LEGENDplex ™ Human CD8/NK | BioLegend | #741186 |
| | Panel (13-plex) | | |

### REFERENCES

Aghajanian, H., Kimura, T., Rurik, J. G., Hancock, A. S., Leibowitz, M. S., Li, L., Scholler, J., Monslow, J., Lo, A., Han, W., et al. (2019). Targeting cardiac fibrosis with engineered T cells. Nature 573, 430-433.
Amor, C., Feucht, J., Leibold, J., Ho, Y. J., Zhu, C., Alonso-Curbelo, D., Mansilla-Soto, J., Boyer, J. A., Li, X., Giavridis, T., et al. (2020). Senolytic CAR T cells reverse senescence-associated pathologies. Nature 583, 127-132.
Borcherds, W., Bremer, A., Borgia, M. B., and Mittag, T. (2021). How do intrinsically disordered protein regions encode a driving force for liquid-liquid phase separation? Curr Opin Struct Biol 67, 41-50.
Bunnell, S. C., Hong, D. I., Kardon, J. R., Yamazaki, T., McGlade, C. J., Barr, V. A., and Samelson, L. E. (2002). T cell receptor ligation induces the formation of dynamically regulated signaling assemblies. J Cell Biol 158, 1263-1275.
Case, L. B., Ditlev, J. A., and Rosen, M. K. (2019). Regulation of Transmembrane Signaling by Phase Separation. Annu Rev Biophys 48, 465-494.
Chen, J., Qiu, S., Li, W., Wang, K., Zhang, Y., Yang, H., Liu, B., Li, G., Li, L., Chen, M., et al. (2023). Tuning charge density of chimeric antigen receptor optimizes tonic signaling and CAR-T cell fitness. Cell Res 33, 341-354.
Chong, S., Dugast-Darzacq, C., Liu, Z., Dong, P., Dailey, G. M., Cattoglio, C., Heckert, A., Banala, S., Lavis, L., Darzacq, X., and Tjian, R. (2018). Imaging dynamic and selective low-complexity domain interactions that control gene transcription. Science 361.
Deng, Y., Liu, J., Zheng, Q., Eliezer, D., Kallenbach, N. R., and Lu, M. (2006). Antiparallel four-stranded coiled coil specified by a 3-3-1 hydrophobic heptad repeat. Structure 14, 247-255.
Dong, R., Libby, K. A., Blaeschke, F., Fuchs, W., Marson, A., Vale, R. D., and Su, X. (2020). Rewired signaling network in T cells expressing the chimeric antigen receptor (CAR). EMBO J 39, e104730.
Dzuricky, M., Rogers, B. A., Shahid, A., Cremer, P. S., and Chilkoti, A. (2020). De novo engineering of intracellular condensates using artificial disordered proteins. Nat Chem 12, 814-825.
Elinav, E., Waks, T., and Eshhar, Z. (2008). Redirection of regulatory T cells with predetermined specificity for the treatment of experimental colitis in mice. Gastroenterology 134, 2014-2024.
Ellebrecht, C. T., Bhoj, V. G., Nace, A., Choi, E. J., Mao, X., Cho, M. J., Di Zenzo, G., Lanzavecchia, A., Seykora, J. T., Cotsarelis, G., et al. (2016). Reengineering chimeric antigen receptor T cells for targeted therapy of autoimmune disease. Science 353, 179-184.
Ghobadi, A. (2018). Chimeric antigen receptor T cell therapy for non-Hodgkin lymphoma. Curr Res Transl Med 66, 43-49.
Gudipati, V., Rydzek, J., Doel-Perez, I., Goncalves, V. D. R., Scharf, L., Konigsberger, S., Lobner, E., Kunert, R., Einsele, H., Stockinger, H., et al. (2020). Inefficient CAR-proximal signaling blunts antigen sensitivity. Nat Immunol 21, 848-856.
Hale, M., Mesojednik, T., Romano Ibarra, G. S., Sahni, J., Bernard, A., Sommer, K., Scharenberg, A. M., Rawlings, D. J., and Wagner, T. A. (2017). Engineering HIV-Resistant, Anti-HIV Chimeric Antigen Receptor T Cells. Mol Ther 25, 570-579.
Heitzeneder, S., Bosse, K. R., Zhu, Z., Zhelev, D., Majzner, R. G., Radosevich, M. T., Dhingra, S., Sotillo, E., Buongervino, S., Pascual-Pasto, G., et al. (2022). GPC2-CAR T cells tuned for low antigen density mediate potent activity against neuroblastoma without toxicity. Cancer Cell 40, 53-69 e59.
Hofman, E. G., Bader, A. N., Voortman, J., van den Heuvel, D. J., Sigismund, S., Verkleij, A. J., Gerritsen, H. C., and van Bergen en Henegouwen, P. M. (2010). Ligand-induced EGF receptor oligomerization is kinase-dependent and enhances internalization. J Biol Chem 285, 39481-39489.
Houtman, J. C., Yamaguchi, H., Barda-Saad, M., Braiman, A., Bowden, B., Appella, E., Schuck, P., and Samelson, L. E. (2006). Oligomerization of signaling complexes by the multipoint binding of GRB2 to both LAT and SOS1. Nat Struct Mol Biol 13, 798-805.
Huang, J., Brameshuber, M., Zeng, X., Xie, J., Li, Q. J., Chien, Y. H., Valitutti, S., and Davis, M. M. (2013). A single peptide-major histocompatibility complex ligand triggers digital cytokine secretion in CD4(+) T cells. Immunity 39, 846-857.
Huang, W. Y. C., Alvarez, S., Kondo, Y., Lee, Y. K., Chung, J. K., Lam, H. Y. M., Biswas, K. H., Kuriyan, J., and Groves, J. T. (2019). A molecular assembly phase transition and kinetic proofreading modulate Ras activation by SOS. Science 363, 1098-1103.
Irgen-Gioro, S., Yoshida, S., Walling, V., and Chong, S. (2022). Fixation can change the appearance of phase separation in living cells. Elife 11.
Irvine, D. J., Purbhoo, M. A., Krogsgaard, M., and Davis, M. M. (2002). Direct observation of ligand recognition by T cells. Nature 419, 845-849.
James, S. E., Greenberg, P. D., Jensen, M. C., Lin, Y., Wang, J., Till, B. G., Raubitschek, A. A., Forman, S. J., and Press, O. W. (2008). Antigen sensitivity of CD22-specific chimeric TCR is modulated by target epitope distance from the cell membrane. J Immunol 180, 7028-7038.
Kato, M., Han, T. W., Xie, S., Shi, K., Du, X., Wu, L. C., Mirzaei, H., Goldsmith, E. J., Longgood, J., Pei, J., et al. (2012). Cell-free formation of RNA granules: low complexity sequence domains form dynamic fibers within hydrogels. Cell 149, 753-767.
Katsarou, A., Sjostrand, M., Naik, J., Mansilla-Soto, J., Kefala, D., Kladis, G., Nianias, A., Ruiter, R., Poels, R., Sarkar, I., et al. (2021). Combining a CAR and a chimeric costimulatory receptor enhances T cell sensitivity to low antigen density and promotes persistence. Sci Transl Med 13, eabh1962.
Lee, D. W., Kochenderfer, J. N., Stetler-Stevenson, M., Cui, Y. K., Delbrook, C., Feldman, S. A., Fry, T. J., Orentas, R., Sabatino, M., Shah, N. N., et al. (2015). T cells expressing CD19 chimeric antigen receptors for acute lymphoblastic leukaemia in children and young adults: a phase 1 dose-escalation trial. Lancet 385, 517-528.
Leibman, R. S., Richardson, M. W., Ellebrecht, C. T., Maldini, C. R., Glover, J. A., Secreto, A. J., Kulikovskaya, I., Lacey, S. F., Akkina, S. R., Yi, Y., et al. (2017). Supraphysiologic control over HIV-1 replication mediated by CD8 T cells expressing a re-engineered CD4-based chimeric antigen receptor. PLoS Pathog 13, e1006613.
Li, P., Banjade, S., Cheng, H. C., Kim, S., Chen, B., Guo, L., Llaguno, M., Hollingsworth, J. V., King, D. S., Banani, S. F., et al. (2012). Phase transitions in the assembly of multivalent signalling proteins. Nature 483, 336-340.
Li, R., Li, T., Lu, G., Cao, Z., Chen, B., Wang, Y., Du, J., and Li, P. (2022). Programming cell-surface signaling by phase-separation-controlled compartmentalization. Nat Chem Biol 18, 1351-1360.
Long, A. H., Haso, W. M., Shern, J. F., Wanhainen, K. M., Murgai, M., Ingaramo, M., Smith, J. P., Walker, A. J., Kohler, M. E., Venkateshwara, V. R., et al. (2015). 4-1BB costimulation ameliorates T cell exhaustion induced by tonic signaling of chimeric antigen receptors. Nat Med 21, 581-590.
Majzner, R. G., and Mackall, C. L. (2018). Tumor Antigen Escape from CAR T-cell Therapy. Cancer Discov 8, 1219-1226.
Majzner, R. G., Rietberg, S. P., Sotillo, E., Dong, R., Vachharajani, V. T., Labanieh, L., Myklebust, J. H., Kadapakkam, M., Weber, E.W., Tousley, A. M., et al. (2020). Tuning the Antigen Density Requirement for CAR T Cell Activity. Cancer Discov.
Maldini, C. R., Ellis, G. I., and Riley, J. L. (2018). CAR T cells for infection, autoimmunity and allotransplantation. Nat Rev Immunol 18, 605-616.
Mansilla-Soto, J., Eyquem, J., Haubner, S., Hamieh, M., Feucht, J., Paillon, N., Zucchetti, A. E., Li, Z., Sjostrand, M., Lindenbergh, P. L., et al. (2022). HLA-independent T cell receptors for targeting tumors with low antigen density. Nat Med 28, 345-352.
Maude, S. L., Laetsch, T. W., Buechner, J., Rives, S., Boyer, M., Bittencourt, H., Bader, P., Verneris, M. R., Stefanski, H. E., Myers, G. D., et al. (2018). Tisagenlecleucel in Children and Young Adults with B-Cell Lymphoblastic Leukemia. N Engl J Med 378, 439-448.
Pappu, R. V., Cohen, S. R., Dar, F., Farag, M., and Kar, M. (2023). Phase Transitions of Associative Biomacromolecules. Chem Rev.
Park, J. H., Riviere, I., Gonen, M., Wang, X., Senechal, B., Curran, K. J., Sauter, C., Wang, Y., Santomasso, B., Mead, E., et al. (2018). Long-Term Follow-up of CD19 CAR Therapy in Acute Lymphoblastic Leukemia. N Engl J Med 378, 449-459.
Pedelacq, J. D., Cabantous, S., Tran, T., Terwilliger, T. C., and Waldo, G. S. (2006). Engineering and characterization of a superfolder green fluorescent protein. Nat Biotechnol 24, 79-88.
Porter, D. L., Levine, B. L., Kalos, M., Bagg, A., and June, C. H. (2011). Chimeric antigen receptor-modified T cells in chronic lymphoid leukemia. N Engl J Med 365, 725-733.
Pozniak, M., Sokolowska-Wedzina, A., Jastrzebski, K., Szymczyk, J., Porebska, N., Krzyscik, M. A., Zakrzewska, M., Miaczynska, M., Otlewski, J., and Opalinski, L. (2020). FGFR1 clustering with engineered tetravalent antibody improves the efficiency and modifies the mechanism of receptor internalization. Mol Oncol 14, 1998-2021.
Priceman, S. J., Gerdts, E. A., Tilakawardane, D., Kennewick, K. T., Murad, J. P., Park, A. K., Jeang, B., Yamaguchi, Y., Yang, X., Urak, R., et al. (2018). Co-stimulatory signaling determines tumor antigen sensitivity and persistence of CAR T cells targeting PSCA+ metastatic prostate cancer. Oncoimmunology 7, e1380764.
Protter, D. S. W., Rao, B. S., Van Treeck, B., Lin, Y., Mizoue, L., Rosen, M. K., and Parker, R. (2018). Intrinsically Disordered Regions Can Contribute Promiscuous Interactions to RNP Granule Assembly. Cell Rep 22, 1401-1412.
Salter, A. I., Rajan, A., Kennedy, J. J., Ivey, R. G., Shelby, S. A., Leung, I., Templeton, M. L., Muhunthan, V., Voillet, V., Sommermeyer, D., et al. (2021). Comparative analysis of TCR and CAR signaling informs CAR designs with superior antigen sensitivity and in vivo function. Sci Signal 14.
Spencer, R. K., and Hochbaum, A. I. (2017). The Phe-Ile Zipper: A Specific Interaction Motif Drives Antiparallel Coiled-Coil Hexamer Formation. Biochemistry 56, 5300-5308.
Su, X., Ditlev, J. A., Hui, E., Xing, W., Banjade, S., Okrut, J., King, D. S., Taunton, J., Rosen, M. K., and Vale, R. D. (2016). Phase separation of signaling molecules promotes T cell receptor signal transduction. Science 352, 595-599.
Wang, J., Choi, J. M., Holehouse, A. S., Lee, H. O., Zhang, X., Jahnel, M., Maharana, S., Lemaitre, R., Pozniakovsky, A., Drechsel, D., et al. (2018). A Molecular Grammar Governing the Driving Forces for Phase Separation of Prion-like RNA Binding Proteins. Cell 174, 688-699 e616.
Watanabe, K., Terakura, S., Martens, A. C., van Meerten, T., Uchiyama, S., Imai, M., Sakemura, R., Goto, T., Hanajiri, R., Imahashi, N., et al. (2015). Target antigen density governs the efficacy of anti-CD20-CD28-CD3 zeta chimeric antigen receptor-modified effector CD8+ T cells. J Immunol 194, 911-920.
Wilkens, A. B., Fulton, E. C., Pont, M. J., Cole, G. O., Leung, I., Stull, S. M., Hart, M. R., Bernstein, I.D., Furlan, S. N., and Riddell, S. R. (2022). NOTCH1 signaling during CD4+ T-cell activation alters transcription factor networks and enhances antigen responsiveness. Blood 140, 2261-2275.
Xiao, Q., McAtee, C. K., and Su, X. (2022a). Phase separation in immune signalling. Nat Rev Immunol 22, 188-199.
Xiao, Q., Zhang, X., Tu, L., Cao, J., Hinrichs, C. S., and Su, X. (2022b). Size-dependent activation of CAR-T cells. Sci Immunol 7, eab13995.
Xu, W., Pei, G., Liu, H., Ju, X., Wang, J., Ding, Q., and Li, P. (2021). Compartmentalization-aided interaction screening reveals extensive high-order complexes within the SARS-CoV-2 proteome. Cell Rep 37, 109778.
You, K., Huang, Q., Yu, C., Shen, B., Sevilla, C., Shi, M., Hermjakob, H., Chen, Y., and Li, T. (2020). PhaSepDB: a database of liquid-liquid phase separation related proteins. Nucleic Acids Res 48, D354-D359.
Zeng, L., Palaia, I., Saric, A., and Su, X. (2021). PLCgamma1 promotes phase separation of T cell signaling components. J Cell Biol 220.
Zhang, W., Sloan-Lancaster, J., Kitchen, J., Trible, R. P., and Samelson, L. E. (1998). LAT: the ZAP-70 tyrosine kinase substrate that links T cell receptor to cellular activation. Cell 92, 83-92.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

1. A polypeptide comprising
(a) an amino acid sequence of an intrinsically disordered region (IDR) of a protein, optionally comprising between 50 and 500 amino acids, or a functional fragment or variant thereof; and
(b) an amino acid sequence that is heterologous to the IDR.

2. The polypeptide of claim 1, wherein the amino acid sequence of the IDR comprises any one of SEQ ID NOs:1-6, or a functional variant thereof having an amino acid sequence at least 70%, 75%, 80%, 85%, 90%, 95%, or 99% identical to any one of SEQ ID NOs:1-6.

3. The polypeptide of claim 1, wherein the amino acid sequence of the IDR comprises at least 70% and less than 100% sequence identity to any one of SEQ ID NOs:1-6, or a homologue thereof, or functional fragment of the foregoing.

4. The polypeptide of claim 1, wherein the heterologous sequence comprises one or more of a chimeric antigen receptor (CAR), protein transduction domain, fusogenic polypeptide, targeting signal, expression, and/or purification tag.

5. The polypeptide of claim 4, wherein the heterologous sequence comprises a chimeric antigen receptor (CAR), and wherein the amino acid sequence of the IDR is present within the extracellular or intracellular region of the CAR.

6. The polypeptide of claim 4, wherein the heterologous sequence comprises a chimeric antigen receptor (CAR), and wherein the amino acid sequence of the IDR is contiguous with the carboxyl terminus or amino terminus of the CAR, or inserted between two domains of the CAR, optionally wherein the polypeptide comprises a structure according to:
(i) N-[CAR]-[IDR]_{Z}-C, where “N” and “C” refer to the amino (NH₂) and Carboxyl (COOH) termini, respectively, “CAR” refers to a single contiguous CAR fusion protein, “IDR” refers to an IDR and “Z” is an integer between one and four, inclusive; or
(ii) N-[CAR part 1]-[IDR]_{Z}-[CAR part 2], where “N” and “C” refer to the amino (NH₂) and Carboxyl (COOH) termini, respectively, “IDR” refers to an IDR and “Z” is an integer between one and four, inclusive, “CAR-part 1” includes one or more traditional CAR domains and “CAR-part 2” includes one or more traditional CAR domains such that without the intervening [IDR]_{Z} domain, CAR-part 1 and CAR-part 2 form a single contiguous CAR fusion protein.

7. The polypeptide of claim 4, wherein the heterologous sequence comprises a chimeric antigen receptor (CAR) comprising a transmembrane domain of CD8a, cytosolic signaling domains or a fragment there of derived from CD28, 41BB, and CD3 zeta, and optionally wherein the amino acid sequence of the IDR is contiguous with the intracellular component of CD3 zeta.

8. The polypeptide of claim 4, wherein the CAR is specific for an antigen selected from the group consisting of a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

9. The polypeptide of claim 8, wherein the CAR targets one or more antigens selected from the group consisting of AFP, AKAP 4, ALK, Androgen receptor, B7H3, BCMA, Bcr Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD2, GD3, GloboH, GM3, gp100, GPC3, HER 2/neu, HER2, HMWMAA, HPV E6/E7, hTERT, Idiotype, IL12, IL13RA2, IM19, IX, LCK, Legumain, IgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR R, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, XAGE, Claudin-6, Claudin-18.2 and CD70.

10. The polypeptide of claim 8, wherein the antigen is a cancer antigen selected from the group consisting of 41BB, 5T4, adenocarcinoma antigen, alpha fetoprotein, BAFF, B lymphoma cell, C242 antigen, CA 125, carbonic anhydrase 9 (CA IX), C MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD2, GD3 ganglioside, glycoprotein 75, GPNMB, HER2/neu, HGF, human scatter factor receptor kinase, IGF 1 receptor, IGF I, IgGl, L1 CAM, IL 13, IL 6, insulin-like growth factor I receptor, integrin α5β1, integrin αvβ3, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF β, TRAIL R1, TRAIL R2, tumor antigen CTAA16.88, VEGF A, VEGFR 1, VEGFR2, and vimentin.

11. The polypeptide of claim 10, wherein the CAR is anti-CD19, anti-CD22, or anti-HER2.

12. The polypeptide of claim 1, comprising the polypeptide sequence of any one of SEQ ID NOs:7-27.

13. The polypeptide of claim 1, lacking a coiled-coiled domain.

14. A nucleic acid comprising a nucleic acid encoding the polypeptide of claim 1.

15.-23. (canceled)

24. An isolated cell comprising the polypeptide of claim 1.

25.-27. (canceled)

28. The isolated cell of claim 24, wherein the polypeptide comprises
(i) an amino acid sequence encoding a CAR; and
(ii) an amino acid sequence of one or more of SEQ ID NOs:1-6, or a variant having at least 70%, 75%, 80%, 85%, 90%, 95%, or 99% identity to one or more of SEQ ID NOs:1-6.

29.-31. (canceled)

32. A pharmaceutical composition comprising a population of cells comprising a plurality of the cell of claim 24 and a pharmaceutically acceptable buffer, carrier, diluent, or excipient.

33. A method of treating a subject having a disease, disorder, or condition comprising administering to the subject an effective amount of the pharmaceutical composition of claim 32.

34. A method of treating a subject having a disease, disorder, or condition associated with an elevated expression or specific expression of an antigen, the method comprising administering to the subject an effective amount of a cell of claim 28, wherein the CAR targets the antigen.

35.-39. (canceled)

40. A chimeric antigen receptor (CAR) including a polypeptide of an intrinsically disordered region (IDR) (CAR-IDR), wherein
(i) the CAR-IDR targets CD19 and comprises the amino acid sequence of any one of SEQ ID NOs:7-18;
(ii) the CAR-IDR targets CD22 and comprises the amino acid sequence of any one of SEQ ID NOs: 20-22; or
(iii) the CAR-IDR targets HER2 and comprises the amino acid sequence of any one of SEQ ID NOs:24-26.

41.-52. (canceled)
